SEQUENCE LISTING (58 sequences)

SEQ ID NO: 1 - DNA, 1414 bp, Homo sapiens
tttcgtgaggaagttcaagggcgagagtgagtaccagcagaaggctgggagtctgtagtttgttcctgctgccaggctccactgaggggaacggggacctgtctgaagagaagatgcccctgctgacactctacctgctcctcttctggctctcaggctactccattgtcactcaaatcaccggtccaacaacagtgaatggcttggagcggggctccttgaccgtgcagtgtgtttacagatcaggctgggagacctacttgaagtggtggtgtcgaggagctatttggcgtgactgcaagatccttgttaaaaccagtgggtcagagcaggaggtgaagagggaccgggtgtccatcaaggacaatcagaaaaaccgcacgttcactgtgaccatggaggatctcatgaaaactgatgctgacacttactggtgtggaattgagaaaactggaaatgaccttggggtcacagttcaagtgaccattgacccagcgtcgactcctgcccccaccacgcctacctccactacgtttacagcaccagtcacccaagaagaaactagcagctccccaactctgaccggccaccacttggacaacaggcacaagctcctgaagctcagtgtcctcctgcccctcatcttcaccatattgctgctgcttttggtggccgcctcactcttggcttggaggatgatgaagtaccagcagaaagcagccgggatgtccccagagcaggtactgcagcccctggagggcgacctctgctatgcagacctgaccctgcagctggccggaacctccccgcgaaaggctaccacgaagctttcctctgcccaggttgaccaggtggaagtggaatatgtcaccatggcttccttgccgaaggaggacatttcctatgcatctctgaccttgggtgctgaggatcaggaaccgacctactgcaacatgggccacctcagtagccacctccccggcaggggccctgaggagcccacggaatacagcaccatcagcaggccttagcctgcactccaggctccttcttggaccccaggctgtgagcacactcctgcctcatcgaccgtctgccccctgctcccctcatcaggaccaacccggggactggtgcctctgcctgatcagccagcattgcccctagctctgggttgggcttggggccaagtctcagggggcttctaggagttggggttttctaaacgtcccctcctctcctacatagttgaggagggggctagggatatgctctggggctttcatgggaatgatgaagatgataatgagaaaaatgttatcattattatcatgaagtaccattatcataatacaatgaacctttatttattgcctaccacatgttatgggctgaataatggcccccaaagata

SEQ ID NO: 2 - DNA, 1414 bp, Homo sapiens; identical to SEQ ID NO: 1
  CDS (114)..(1031)

SEQ ID NO: 3 - protein, 305 aa, Homo sapiens
MPLLTLYLLLFWLSGYSIVTQITGPTTVNGLERGSLTVQCVYRSGWETYLKWWCRGAIWRDCKILVKTSGSEQEVKRDRVSIKDNQKNRTFTVTMEDLMKTDADTYWCGIEKTGNDLGVTVQVTIDPASTPAPTTPTSTTFTAPVTQEETSSSPTLTGHHLDNRHKLLKLSVLLPLIFTILLLLLVAASLLAWRMMKYQQKAAGMSPEQVLQPLEGDLCYADLTLQLAGTSPRKATTKLSSAQVDQVEVEYVTMASLPKEDISYASLTLGAEDQEPTYCNMGHLSSHLPGRGPEEPTEYSTISRP

SEQ ID NO: 4 - protein, 17 aa, Homo sapiens
MPLLTLYLLLFWLSGYS

SEQ ID NO: 5 - protein, 78 aa, Homo sapiens
RGSLTVQCVYRSGWETYLKWWCRGAIWRDCKILVKTSGSEQEVKRDRVSIKDNQKNRTFTVTMEDLMKTDADTYWCGI

SEQ ID NO: 6 - protein, 23 aa, Homo sapiens
SVLLPLIFTILLLLLVAASLLAW

SEQ ID NO: 7 - protein, 154 aa, Homo sapiens
MIVTQITGPTTVNGLERGSLTVQCVYRSGWETYLKWWCRGAIWRDCKILVKTSGSEQEVKRDRVSIKDNQKNRTFTVTMEDLMKTDADTYWCGIEKTGNDLGVTVQVTIDPASTPAPTTPTSTTFTAPVTQEETSSSPTLTGHHLDNRHKLLKL

SEQ ID NO: 8 - protein, 290 aa, Homo sapiens
MPLLTLYLLLFWLSGYSIATQITGPTTVNGLERGSLTVQCVYRSGWETYLKWWCRGAIWRDCKILVKTSGSEQEVKRDRVSIKDNQKNRTFTVTMEDLMKTDADTYWCGIEKTGNDLGVTVQVTIDPAPVTQEETSSFPTLTGHHLDNRHKLLKLSVLLPLIFTILLLLLVAASLLAWRMMKYQQKAAGMSPEQVLQPLEGDLCYADLTLQLAGTSPRKATTKLSSAQVDQVEVEYVTMASLPKEDISYASLTLGAEDQEPTYCNMGHLSSHLPGRGPEEPTEYSTISRP

SEQ ID NO: 9 - protein, 194 aa, Homo sapiens [sequence omitted]

SEQ ID NO: 10 - protein, 6 aa, Artificial Sequence
  Consensus Sequence
  MISC_FEATURE (1)..(6): X1= I, L, or V; X2= any amino acid, X4= any amino acid; X5 =any amino acid;  X6= L or V
XXYXXX

SEQ ID NO: 11 - protein, 12 aa, Homo sapiens
EEPTEYSTISRP

SEQ ID NO: 12 - DNA, 1804 bp, Homo sapiens
  CDS (280)..(768)
cccgggtcgaccatttcgtggcaaaagtggctccaggtgcagaaggttggagttcagtggtgcgatctgggttcactgcaacctctgccgcctgggttcaagtgattctcctacctcggcctcccgagtcactgggattacagtctctttcacccatgggaagtgactgaaggaggaagttcaagggcgagagtgagtaccagcagaaggctgggagtctgtagtttgttcctgctgccaggctccactgaggggaacggggacctgtctgaagagaagatgcccctgctgacactctacctgctcctcttctggctctcaggctactccattgccactcaaatcaccggtccaacaacagtgaatggcttggagcggggctccttgaccgtgcagtgtgtttacagatcaggctgggagacctacttgaagtggtggtgtcgaggagctatttggcgtgactgcaagatccttgttaaaaccagtgggtcagagcaggaggtgaagagggaccgggtgtccatcaaggacaatcagaaaaaccgcacgttcactgtgaccatggaggatctcatgaaaactgatgctgacacttactggtgtggaattgagaaaactggaaatgaccttggggtcacagttcaagtgaccattgacccagcaccagtcacccaagaagaaactagcagctccccaactctgaccggccaccacttggacaacaggtactgcagcccctggagggcgacctctgctatgcagacctgaccctgcagctggccggaacctccccgcaaaaggctaccacgaagctttcctctgcccaggttgaccaggtggaagtggaatatgtcaccatggcttccttgccgaaggaggacatttcctatgcatctctgaccttgggtgctgaggatcaggaaccgacctactgcaacatgggccacctcagtagccacctccccggcaggggccctgaggagcccacggaatacagcaccatcagcaggccttagcctgcactccaggctccttcttggaccccaggctgtgagcacactcctgcctcatcgaccgtctgccccctgctcccctcatcaggaccaacccggggactggtgcctctgcctgatcagccagcattgcccctagctctgggttgggcttggggccaagtctcagggggcttctaggagttggggttttctaaacgtcccctcctctcctacatagttgaggagggggctagggatatgctctggggctttcatgggaatgatgaagatgataatgagaaaaatgttatcattattatcatgaagtaccattatcgtaatacaatgaacctttatttattgcctaccacatgttatgggctgaataatggcccccaaagatatctgtgtcctaatcctcagaacctgtgactgttaccttctgtggcagaaagggacagtgcagatgtatgtaagttaaggactttgagatagagaggttattcttgctgattcaggtgggcccaaaatatcaccacaagggtcctcataagaaagaggccagaaggtcaaagaggtagagacaaagtgatgatggaagtggacgtgggtgtgacgtgagcaggggccatgaatgccgcagccttcagatgccagaaagggaaaggaatggattcccctgcctggagcctccaaaagaaaccagccctgcccacgccttgacttgagcccattgaaactgatcttgagctcctggcctccagaattgcaggagaataaatttgtgttgtttttaatgaaaaaaaaaa

SEQ ID NO: 13 - protein, 162 aa, Homo sapiens
MPLLTLYLLLFWLSGYSIATQITGPTTVNGLERGSLTVQCVYRSGWETYLKWWCRGAIWRDCKILVKTSGSEQEVKRDRVSIKDNQKNRTFTVTMEDLMKTDADTYWCGIEKTGNDLGVTVQVTIDPAPVTQEETSSSPTLTGHHLDNRYCSPWRATSAMQT

SEQ ID NO: 14 - DNA, 1770 bp, Homo sapiens
  CDS (108)..(989)
gaggaagttcaagggcgagagtgagtaccagcagaaggctgggagtctgtagtttgttcctgctgccaggctccactgaggggaacggggacctgtctgaagagaagatgcccctgctgacactctacctgctcctcttctggctctcaggctactccattgccactcaaatcaccggtccaacaacagtgaatggcttggagcggggctccttgaccgtgcagtgtgtttacagatcaggctgggagacctacttgaagtggtggtgtcgaggagctatttggcgtgactgcaagatccttgttaaaaccagtgggtcagagcaggaggtgaagagggaccgggtgtccatcaaggacaatcagaaaaaccgcacgttcactgtgaccatggaggatctcatgaaaactgatgctgacacttactggtgtggaattgagaaaactggaaatgaccttggggtcacagttcaagtgaccattgacccagcaccagtcacccaagaagaaactagcagctccccaactctgaccggccaccacttggacaacaggcacaagctcctgaagctcagtgtcctcctgcccctcatcttcaccatattgctgctgcttttggtggccgcctcactcttggcttggaggatgatgaagtaccagcagaaagcagccgggatgtccccagagcaggtactgcagcccctggagggcgacctctgctatgcagacctgaccctgcagctggccggaacctccccgcgaaaggctaccacgaagctttcctctgcccaggttgaccaggtggaagtggaatatgtcaccatggcttccttgccgaaggaggacatttcctatgcatctctgaccttgggtgctgaggatcaggaaccgacctactgcaacatgggccacctcagtagccacctccccggcaggggccctgaggagcccacggaatacagcaccatcagcaggccttagcctgcactccaggctccttcttggaccccaggctgtgagcacactcctgcctcatcgaccgtctgccccctgctcccctcatcaggaccaacccggggactggtgcctctgcctgatcagccagcattgcccctagctctgggttgggcttggggccaagtctcagggggcttctaggagttggggttttctaaacgtcccctcctctcctacatagttgaggagggggctagggatatgctctggggctttcatgggaatgatgaagatgataatgagaaaaatgttatcattattatcatgaagtaccattatcataatacaatgaacctttatttattgcctaccacatgttatgggctgaataatggcccccaaagatatctgtgtcctaatcctcagaacttgtgactgttaccttctgtggcagaaagggacagtgcagatgtatgtaagttaaggactttgagatagagaggttattcttgctgattcaggtgggcccaaaatatcaccacaagggtcctcataagaaagaggccagaaggtcaaagaggtagagacaaagtgatgatggaagtggacgtgggtgtgacgtgagcaggggccatgaatgccgcagccttcagatgccagaaagggaaaggaatggattcccctgcctggagcctccaaaagaaaccagccctgcccacgccttgacttgagcccattgaaactgatcttgagctcctggcctccagaattgcaggagaataaatttgtgttgtttttaatgagccaaaaaaaa

SEQ ID NO: 15 - protein, 290 aa, Homo sapiens [sequence omitted]

SEQ ID NO: 16 - DNA, 1657 bp, Homo sapiens
  CDS (108)..(683)
gaggaagttcaagggcgagagtgagtaccagcagaaggctgggagtctgtagtttgttcctgctgccaggctccactgaggggaacggggacctgtctgaagagaagatgcccctgctgacactctacctgctcctcttctggctctcaggctactccattgccactcaaatcaccggtccaacaacagtgaatggcttggagcggggctccttgaccgtgcagtgtgtttacagatcaggctgggagacctacttgaagtggtggtgtcgaggagctatttggcgtgactgcaagatccttgttaaaaccagtgggtcagagcaggaggtgaagagggaccgggtgtccatcaaggacaatcagaaaaaccgcacgttcactgtgaccatggaggatctcatgaaaactgatgctgacacttactggtgtggaattgagaaaactggaaatgaccttggggtcacagttcaagtgaccattgacccagcaccagtcacccaagaagaaactagcagctccccaactctgaccggccaccacttggacaacagcagccgggatgtccccagagcaggtactgcagcccctggagggcgacctctgctatgcagacctgaccctgcagctggccggaacctccccgcgaaaggctaccacgaagctttcctctgcccaggttgaccaggtggaagtggaatatgtcaccatggcttccttgccgaaggaggacatttcctatgcatctctgaccttgggtgctgaggatcaggaaccgacctactgcaacatgggccacctcagtagccacctccccggcaggggccctgaggagcccacggaatacagcaccatcagcaggccttagcctgcactccaggctccttcttggaccccaggctgtgagcacactcctgcctcatcgaccgtctgccccctgctcccctcatcaggaccaacccggggactggtgcctctgcctgatcagccagcattgcccctagctctgggttgggcttggggccaagtctcagggggcttctaggagttggggttttctaaacgtcccctcctctcctacatagttgaggagggggctagggatatgctctggggctttcatgggaatgatgaagatgataatgagaaaaatgttatcattattatcatgaagtaccattatcataatacaatgaacctttatttattgcctaccacatgttatgggctgaataatggcccccaaagatatctgtgtcctaatcctcagaacttgtgactgttaccttctgtggcagaaagggacagtgcagatgtatgtaagttaaggactttgagatagagaggttattcttgctgattcaggtgggcccaaaatatcaccacaagggtcctcataagaaagaggccagaaggtcaaagaggtagagacaaagtgatgatggaagtggacgtgggtgtgacgtgagcaggggccatgaatgccgcagccttcagatgccagaaagggaaaggaatggattcccctgcctggagcctccaaaagaaaccagccctgcccacgccttgacttgagcccattgaaactgatcttgagctcctggcctccagaattgcaggagaataaatttgtgttgtttttaatgagccaaaaaaaa

SEQ ID NO: 17 - protein, 191 aa, Homo sapiens
MPLLTLYLLLFWLSGYSIATQITGPTTVNGLERGSLTVQCVYRSGWETYLKWWCRGAIWRDCKILVKTSGSEQEVKRDRVSIKDNQKNRTFTVTMEDLMKTDADTYWCGIEKTGNDLGVTVQVTIDPAPVTQEETSSSPTLTGHHLDNSSRDVPRAGTAAPGGRPLLCRPDPAAGRNLPAKGYHEAFLCPG

SEQ ID NO: 18 - DNA, 1012 bp, Homo sapiens
  CDS (108)..(842)
gaggaagttcaagggcgagagtgagtaccagcagaaggctgggagtctgtagtttgttcctgctgccaggctccactgaggggaacggggacctgtctgaagagaagatgcccctgctgacactctacctgctcctcttctggctctcaggctactccattgccactcaaatcaccggtccaacaacagtgaatggcttggagcggggctccttgaccgtgcagtgtgtttacagatcaggctgggagacctacttgaagtggtggtgtcgaggagctatttggcgtgactgcaagatccttgttaaaaccagtgggtcagagcaggaggtgaagagggaccgggtgtccatcaaggacaatcagaaaaaccgcacgttcactgtgaccatggaggacctcatgaaaactgatgctgacacttactggtgtggaattgagaaaactggaaatgaccttggggtcacagttcaagtgaccattgacccagcaccagtcacccaagaagaaactagcagctccccaactctgaccggccaccacttggacaacaggagtgagggttcccaggctgcaaactacagacctgcagctcatcaggcacaagctcctgaagctcagtgtcctcctgcccctcatcttcaccatattgctgctgcttttggtggccgcctcactcttggcttggaggatgatgaagtaccagcagaaagcagccgggatgtccccagagcaggtactgcagcccctggagggcgacctctgctatgcagacctgaccctgcagctggccggaacctccccgcgaaaggctaccacgaagctttcctctgcccaggttgaccaggtggaagtggaatatgtcaccatggcttccttgccgaaggaggacatttcctatgcatctctgaccttgggtgctgaggatcaggaaccgacctactgcaacatgggccacctcagtagccacctccccggcaggggccctgaggagcccacaaagaatacaatat

SEQ ID NO: 19 - protein, 244 aa, Homo sapiens [sequence omitted]

SEQ ID NO: 20 - DNA, 2089 bp, Homo sapiens
  CDS (374)..(1300)
cccgggtcgaccatttcgtggcaaaagtggctccaggtgcagaaggttggagttcagtggtgcgatctgggttcactgcaacctctgccgcctgggttcaagtgattctcctacctcggcctcccgagtcactgggattacagtctctttcacccatgggaagtgactgaaggaggaagttcaagggcgagagtgagtaccagcagaaggctgggagtctgtagtttgttcctgctgccaggctccactgaggggaacggggacctgtctgaagagaagatgcccctgctgacactctacctgctcctcttctggctctcagaaccactagaaatcattaggaccaccaggacagcctctccacagccatctgatgtggctgcctcagctcgacctcatgagggtcatcagtgctaagagtcaaggctactccattgccactcaaatcaccggtccaacaacagtgaatggcttggagcggggctccttgaccgtgcagtgtgtttacagatcaggctgggagacctacttgaagtggtggtgtcgaggagctatttggcgtgactgcaagatccttgttaaaaccagtgggtcagagcaggaggtgaagagggaccgggtgtccatcaaggacaatcagaaaaaccgcacgttcactgtgaccatggaggatctcatgaaaactgatgctgacacttactggtgtggaattgagaaaactggaaatgaccttggggtcacagttcaagtgaccattgacccagcgtcgactcctgcccccaccacgcctacctccactacgtttacagcaccagtcacccaagaagaaactagcagctccccaactctgaccggccaccacttggacaacaggcacaagctcctgaagctcagtgtcctcctgcccctcatcttcaccatattgctgctgcttttggtggccgcctcactcttggcttggaggatgatgaagtaccagcagaaagcagccgggatgtccccagagcaggtactgcagcccctggagggcgacctctgctatgcagacctgaccctgcagctggccggaacctccccgcaaaaggctaccacgaagctttcctctgcccaggttgaccaggtggaagtggaatatgtcaccatggcttccttgccgaaggaggacatttcctatgcatctctgaccttgggtgctgaggatcaggaaccgacctactgcaacatgggccacctcagtagccacctccccggcaggggccctgaggagcccacggaatacagcaccatcagcaggccttagcctgcactccaggctccttcttggaccccaggctgtgagcacactcctgcctcatcgaccgtctgccccctgctcccctcatcaggaccaacccggggactggtgcctctgcctgatcacccagcattgcccctagctctgggttgggcttggggccaagtctcagggggcttctaggagttggggttttctaaacgtcccctcctctcctacatagttgaggagggggctagggatatgctctggggctttcatgggaatgatgaagatgataatgagaaaaatgttatcattattatcatgaagtaccattatcataatacaatgaacctttatttattgcctaccacatgttatgggctgaataatggcccccaaagatatctgtgtcctaatcctcagaacttgtgactgttaccttctgtggcagaaagggacagtgcagatgtatgtaagttaaggactttgagatagagaggttattcttgctgattcaggtgggcccaaaatatcaccacaagggtcctcataagaaagaggccagaaggtcaaagaggtagagacaaagtgatgatggaagtggacgtgggtgtgacgtgagcaggggccatgaatgccgcagccttcagatgccagaaagggaaaggaatggattcccctgcctggagcctccaaaagaaaccagccctgcccacgccttgacttgagcccattgaaactgattttgagctcctggcctccagaattgcaggagaataaatttgtgttgtttttaatgagaaaaaaaaa

SEQ ID NO: 21 - protein, 308 aa, Homo sapiens
MWLPQLDLMRVISAKSQGYSIATQITGPTTVNGLERGSLTVQCVYRSGWETYLKWWCRGAIWRDCKILVKTSGSEQEVKRDRVSIKDNQKNRTFTVTMEDLMKTDADTYWCGIEKTGNDLGVTVQVTIDPASTPAPTTPTSTTFTAPVTQEETSSSPTLTGHHLDNRHKLLKLSVLLPLIFTILLLLLVAASLLAWRMMKYQQKAAGMSPEQVLQPLEGDLCYADLTLQLAGTSPQKATTKLSSAQVDQVEVEYVTMASLPKEDISYASLTLGAEDQEPTYCNMGHLSSHLPGRGPEEPTEYSTISRP

SEQ ID NO: 22 - DNA, 1145 bp, Homo sapiens
  CDS (264)..(1145)
accgacccttcgggtgagcgaagcgtccgccctgaagagaagatgcccctgctgacactctacctgctcctcttctggctctcagaatttccaggctgggcacggtggctcacgcctgtggtcccggcactttgggaggccaaggcgggcggatcgcagggtcaggagatcgagaccatcctggctacggtgaaaccccatctctaccaaagaaccactagaaatcattaggaccaccaggacagcctctccacagccatctgatgtggctgcctcagctcgacctcatgagggtcatcagtgctaagagtcaaggctactccattgtcactcaaatcaccggtccaacaacagtgaatggcttggagcggggctccttgaccgtgcagtgtgtttacagatcaggctgggagacctacttgaagtggtggtgtcgaggagctatttggcgtgactgcaagatccttgttaaaaccagtgggtcagagcaggaggtgaagagggaccgggtgtccatcaaggacaatcagaaaaaccgcacgttcactgtgaccatggaggatctcatgaaaactgatgctgacacttactggtgtggaattgagaaaactggaaatgaccttggggtcacagttcaagtgaccattgacccagcaccagtcacccaagaagaaattagcagctccccaactctgaccggccaccacttggacaacaggcacaagctcttgaagctcagtgtcctcttgcccctcatcttcaccatattgttgctgcttttggtggccgcctcactcttggcttggaggatgatgaagtaccagcagaaagcagccgggatgtccccagagcaggtactgcagcccttggagggcgacctctgctatgcagacctgaccctgcagcaggccggaacctccccgcgaaaggctaccacgaagctttcctctgcccaggttgaccaggtggaagtggaatatgtcaccatggcttccttgccgaaggaggacatttcctatgcatctctgaccttgggtgctgaggatcaggaaccgacctactgcaacatgggccacctcagtagccacctccccggcaggggccctgaggagcccacggaatacagcaccatcagcaggccttag

SEQ ID NO: 23 - protein, 293 aa, Homo sapiens
MWLPQLDLMRVISAKSQGYSIVTQITGPTTVNGLERGSLTVQCVYRSGWETYLKWWCRGAIWRDCKILVKTSGSEQEVKRDRVSIKDNQKNRTFTVTMEDLMKTDADTYWCGIEKTGNDLGVTVQVTIDPAPVTQEEISSSPTLTGHHLDNRHKLLKLSVLLPLIFTILLLLLVAASLLAWRMMKYQQKAAGMSPEQVLQPLEGDLCYADLTLQQAGTSPRKATTKLSSAQVDQVEVEYVTMASLPKEDISYASLTLGAEDQEPTYCNMGHLSSHLPGRGPEEPTEYSTISRP

SEQ ID NO: 24 - DNA, 869 bp, Homo sapiens
  CDS (278)..(862)
tgaaggaggaagttcaagggcgagagtgagtaccagcagaaggctgggagtctgtagtttgttcctgctgccaggctccactgaggggaacggggacctgtctgaagagaagatgcccctgctgacactctacctgctcctcttctggctctcagcacatggagaccagttgccgttgctgttttgatcaacttcaaaggcattcatccagggtggtttattccagaaccactagaaatcattaggaccaccaggacagcctctccacagccatctgatgtggctgcctcagctcgacctcatgagggtcatcagtgctaagagtcaaggctactccattgccactcaaatcaccggtccaacaacagtgaatggcttggagcggggctccttgaccgtgcagtgtgtttacagatcaggctgggagacctacttgaagtggtggtgtcgaggagctatttggcgtgactgcaagatccttgttaaaaccagtgggtcagagcaggaggtgaagagggaccgggtgtccatcaaggacaatcagaaaaaccgcacgttcactgtgaccatggaggatctcatgaaaactgatgctgacacttactggtgtggaattgagaaaactggaaatgaccttggggtcacagttcaagtgaccattgacccagcaccagtcacccaagaagaaactagcagctccccaactctgaccggccaccacttggacaacagcagccgggatgtccccagagcaggtactgcagcccctggagggcgacctctgctatgcagacctgaccctgcagctggccggaacctccccgcgaaaggctaccacgaagctttcctctgcccaggttgaccaagtg

SEQ ID NO: 25 - protein, 194 aa, Homo sapiens [sequence omitted]

SEQ ID NO: 26 - protein, 10 aa, Homo sapiens
PTEYSTISRP

SEQ ID NO: 27 - protein, 10 aa, Homo sapiens
GYHEAFLCPG

SEQ ID NO: 28 - protein, 10 aa, Homo sapiens
PWRATSAMQT

SEQ ID NO: 29 - protein, 10 aa, Homo sapiens
PTLTGHHLDN

SEQ ID NO: 30 - DNA, 1254 bp, Homo sapiens
  CDS (1)..(1254)
atgccgctgctgctactgctgcccctgctgtgggcaggggccctggctatggataagcttattgtcactcaaatcaccggtccaacaacagtgaatggcttggagcggggctccttgaccgtgcagtgtgtttacagatcaggctgggagacctacttgaagtggtggtgtcgaggagctatttggcgtgactgcaagatccttgttaaaaccagtgggtcagagcaggaggtgaagagggaccgggtgtccatcaaggacaatcagaaaaaccgcacgttcactgtgaccatggaggatctcatgaaaactgatgctgacacttactggtgtggaattgagaaaactggaaatgaccttggggtcacagttcaagtgaccattgacccagcgtcgactcctgcccccaccacgcctacctccactacgtttacagcaccagtcacccaagaagaaactagcagctccccaactctgaccggccaccacttggacaacaggcacaagctcctgaagctcggggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 31 - protein, 417 aa, Homo sapiens
MPLLLLLPLLWAGALAMDKLIVTQITGPTTVNGLERGSLTVQCVYRSGWETYLKWWCRGAIWRDCKILVKTSGSEQEVKRDRVSIKDNQKNRTFTVTMEDLMKTDADTYWCGIEKTGNDLGVTVQVTIDPASTPAPTTPTSTTFTAPVTQEETSSSPTLTGHHLDNRHKLLKLGDPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 32 - protein, 38 aa, Homo sapiens
MIVTQITGPTTVNGLERGSLTVQCVYRSGWETYLKWWC

SEQ ID NO: 33 - protein, 64 aa, Homo sapiens
MIVTQITGPTTVNGLERGSLTVQCVYRSGWETYLKWWCRGAIWRDCKILVKTSGSEQEVKRDRV

SEQ ID NO: 34 - protein, 92 aa, Homo sapiens
MIVTQITGPTTVNGLERGSLTVQCVYRSGWETYLKWWCRGAIWRDCKILVKTSGSEQEVKRDRVSIKDNQKNRTFTVTMEDLMKTDADTYWC

SEQ ID NO: 35 - protein, 118 aa, Homo sapiens
MIVTQITGPTTVNGLERGSLTVQCVYRSGWETYLKWWCRGAIWRDCKILVKTSGSEQEVKRDRVSIKDNQKNRTFTVTMEDLMKTDADTYWCGIEKTGNDLGVTVQVTIDPASTPAPT

SEQ ID NO: 36 - protein, 142 aa, Homo sapiens
MIVTQITGPTTVNGLERGSLTVQCVYRSGWETYLKWWCRGAIWRDCKILVKTSGSEQEVKRDRVSIKDNQKNRTFTVTMEDLMKTDADTYWCGIEKTGNDLGVTVQVTIDPASTPAPTTPTSTTFTAPVTQEETSSSPTLTG

SEQ ID NO: 37 - protein, 24 aa, Homo sapiens
TPTSTTFTAPVTQEETSSSPTLTG

SEQ ID NO: 38 - DNA, 3500 bp, Homo sapiens
  CDS (120)..(737)
gggagaacacaaaggaaacttggacaagtagaaagtggatgacccaggctccgttacatatacttggattccagctgggacctagatttgctgaggacggaagccaaggagacaggaacatgtggctgctcccagctctactccttctctgcctctcaggctgtttgtctctgaagggccccggctctgtgactggcactgcgggggactctctgacagtgtggtgtcagtatgagagcatgtacaagggatataacaagtactggtgccgaggacagtacgacacgtcatgtgagagcattgtggagaccaagggagaagagaaggtggagaggaatggccgcgtgtccatcagagaccacccggaggctctcgccttcactgtgaccatgcagaacctcaatgaagatgatgctggatcttactggtgcaaaattcagacagtgtgggtcctggattcatggtcacgcgatccctcggacctggttagggtgtatgtttccccagcaattacaaccccaaggaggaccacacatccagccacacctcccatcttcctggtggtgaaccctgggcgaaacctcagcaccggggaggtgttgacccaaaattcagggttccggctcagcagccctcacttcctgctcgtggtccttctgaagctgcccctgctcctgagcatgctgggtgctgtcttctgggtgaacaggcctcagtgggctcctcctggaagatagagccaggctgacccgaagccctgcagagccccatccccaggagtgccccgtgcagggagtcaacctttcagactggatgcgagagacctgtggactctggagggatttattgttcctgtgcctcaaaggagagtcctggctcttagaggacactcctctggagtcctcaatgtgcagcttgggggtcctcctttctcctccccagtcccctgcctccctgccctcatgcccacttcccaagggcaagaattccctggaagttggccgggcaccatagcgagtgcctataatcccagctcaggaggctgaggcaggaggatcggctgaacgtaagagttccaggccagcctgggcaacatagagagccctgtctcagagagagagagagagacaggcagagagagagagagagagcgagagagagagagagagagagaaaaggacggaggaagggagggaaaaaattttctggaggagcctggcccttcattcttcttggcacaaatgcttctcaggccagtttcccccagggcaataactacctccttcctgcagacaggagtctccctgaggacacacccctcacagccccagcctttaggctcacagagctccatctcttctctgggacaaccccaagggagcttggtcccggcagagcttgctcatgcagttgcccagtgtcccacatgctcagggctcctctcgtcttctgacgccccttgggtctccctggctttaaggccccttctctccctgtaaccaggcagtgtgtggctgggctcagaacatctctgcctgggtgggccatggatgagctgctccaggttaaagttctggggcgagttgaaacagtaaagggagaaggagctggtgggtgcccctgatatcactcctggaattcccccacctggaggtgtccaatgcacaaggtcaacagagtcagttctgtccactttagaagtctggctgagaccacctgttagcaattaaccttttccacaggcagagcagatatcgtttttcaaagacaggatggagggcacagaggtgaaggatacttgtccacacttgaagtgcacagtgctatcaccactactggtgacccctggctcgggaatgagctcagttggttttctgaatgtttgtcaattactctgtttccctaaagtttgtggggaggactcccacagtctgccacaagcaagcatcaccatctccacttgctacctacatctggaccagtgctccccctctgcccccaggcctgagctcatcctgagctagtgatgccagtgtccagagcacatggagtgaccctctgagtgccgagtgcttaggcagagctgccgtgctctgaacttgagctctggtggggctcccagctccatcatgcatggtgactaggggaaagatggggctggcccaggactcatgctgaaagagaaattcaatctaacaaggcccatattaattatattaacccgggaaatatgatgatgagcaagaaaggagcttatgattcattttatgagaaggataagctcccaagtcattacagcataggaagaactctagagccacaagagatgcttcctgatgtcagggggttggtgaggagaatatcatgccaagctggccaggtcagggaaagaaggtgacattcatgttgatgttgaaggaggtataggatttcagcggagcagggatggagcaacagggagagagagaggacagagaaacagcataagtgaaagcagagaggcgggaaataatcttcataacaatagtcacttatagaagacctatcagcaaatggactgtactgttcacatacagatgctcatggtaggatgtgtctaagattccaatggagtgaaattttcctgtggcaggccagatggaactaacctagaagaactcaggtggtggcaggttcaggaggcttaggactcaactcttacttaaactttgattagctatggtgggatggatttgagcagaacaagatgggattgcagatgtgctttaggaggacttgcctagcagatgtagcaaagtctgaaaggaagaatctgggagtccggagtcttaagcctggaaaaccagaggaatcgggcatcattgacagaagtaggaaagtcaagagcaggcttggatgatgcagagcttagttttctgtgtgtttggaatcatcaggcagagaggtctagcagatagctagagatgtgggttagcagagtgttgagggctggagaggaatgtgtgtagagagattaactggagcccagcaagaagtgaaaacatggagggaagagttcagaaccaaggacagagccctcggggcacctgcatttaaagacaaagggtaggaggaagacattgatcaaggtgctggagcagaaacaaacaggtggaagatgagaagtcaagagccatgaatgtttcaaggaggagagcagagctagagaggtggaggaggaccgggattgagaggaggctattgaacttatcattcacaaggtcactaatgaagttcagaagagcagatcaacagagcaatgacagcagaagccagaagagaagagttaagaaagagtagacagatggcagacagcaggtgcaggagaaacaaacagtggcttctccatgctttgaattttcccttctcccttactccctttttttcccctattcttcctttcttttttccctttatttactcattcattcaataaacttttattgtgtgtcaaaaaaaaa

SEQ ID NO: 39 - protein, 205 aa, Homo sapiens
MWLLPALLLLCLSGCLSLKGPGSVTGTAGDSLTVWCQYESMYKGYNKYWCRGQYDTSCESIVETKGEEKVERNGRVSIRDHPEALAFTVTMQNLNEDDAGSYWCKIQTVWVLDSWSRDPSDLVRVYVSPAITTPRRTTHPATPPIFLVVNPGRNLSTGEVLTQNSGFRLSSPHFLLVVLLKLPLLLSMLGAVFWVNRPQWAPPGR

SEQ ID NO: 40 - DNA, 585 bp, Homo sapiens
  CDS (1)..(585)
atgtggctgtccccagctctgctgcttctcatcctcccaggttactccattgccgctaaaatcactggtccaacaacagtgaatggctcggagcagggctcattgactgtgcagtgtgcttatggctcaggctgggagacctacttgaagtggcggtgtcaaggagctgattggaattactgtaacatccttgttaaaacaaatggatcagagcaggaggtaaagaagaatcgagtttccatcagggacaatcagaaaaaccacatgttcaccgtgaccatggagaatctcaaaagagatgatgctgacagttattggtgtgggactgagagacctggaattgatcttggggtcaaagttcaagtgaccattaacccaggcacacaaactgcagtctcagaatggacaaccacaacagcaagcctggctttcacagctgcagccacccagaagaccagcagccccctcaccaggtccccgctcaagagcacccacttcctgttcctgttcctcctggagctgcctctgctcctgagcatgctggggaccgtcctctgggtgaacagaccacaaagaaggtcttga

SEQ ID NO: 41 - protein, 194 aa, Homo sapiens
MWLSPALLLLILPGYSIAAKITGPTTVNGSEQGSLTVQCAYGSGWETYLKWRCQGADWNYCNILVKTNGSEQEVKKNRVSIRDNQKNHMFTVTMENLKRDDADSYWCGTERPGIDLGVKVQVTINPGTQTAVSEWTTTTASLAFTAAATQKTSSPLTRSPLKSTHFLFLFLLELPLLLSMLGTVLWVNRPQRRS

SEQ ID NO: 42 - DNA, 1348 bp, Homo sapiens
  CDS (210)..(812)
ctcactatagggctcggcgccgccggcaggtcagcaagttcttaaaactgtttcacctgaccacatagtgccccatggccccagcttcctccgtactctcaccagctctgagaaccaaacccagaagaggccagagaaggaaacgagagatgtgagaaggaaaaagagcctcagaccctgctgccacaagggacttccatgctggtgagatgacccagagggctggggctgccatgctgccttcagctctgctccttctctgtgtcccaggctgtctgactgtgagtggccccagcaccgtgatgggcgccgtgggggaatccctgagtgttcagtgtcggtatgaagagaaatacaagacgtttaacaaatactggtgcagacaaccatgcttgccaatttggcatgaaatggtggagaccggagggtctgagggagtggtgaggagtgaccaagtgatcatcacggaccatcctggagacctcaccttcaccgtgaccttggagaacctcacggcagacgatgcaggaaaataccgatgtgggattgcaacaatactgcaggaagatggcctgtctggtttcctgcccgatcccttcttccaggttcaagtgctggtctcatcggcctccagtactgagaactctgtgaagacacctgcatctcccaccaggcccagccaatgccaagggtccctgcccagcagcacctgcttcctgcttctcccactcctgaaggtgcctctgctcctgagcatactcggtgctatcctctgggtgaacaggccttggaggactccttggacagagtcatgaacaggagaacttgcaacaccccatgcccattggaaccctgtccagagacacagcccctctgactgcaaaaaggacttctgaccctgaccctcatatttctttccatcttatcaccggatactttttaaaagttaaaaaaaaatgtaggccgggtgcggtggcttacacctgcaatcccagcactttgggaggccaaggcaggtggatcacttgagtccaggagtttgagagcagcctgggcagcatggtcagacctcatctctacaaaaaaatacaaaaattagcagggtgtggtggtgtatgcctgtgatcccagctacttgggaagctgagacaggaggatcgcttgagccccggagtggaggttgcattgagtcgagattgtgccactgcactccagcctgggtgacagagggagaccctgtctcagataaacaaataaataaataaaatacatcccatacacaagagtatgtatatgaggtatctatacagttcaaggactaaaaataaacatgtgtacccaccattcg

SEQ ID NO: 43 - protein, 201 aa, Homo sapiens
MTQRAGAAMLPSALLLLCVPGCLTVSGPSTVMGAVGESLSVQCRYEEKYKTFNKYWCRQPCLPIWHEMVETGGSEGVVRSDQVIITDHPGDLTFTVTLENLTADDAGKYRCGIATILQEDGLSGFLPDPFFQVQVLVSSASSTENSVKTPASPTRPSQCQGSLPSSTCFLLLPLLKVPLLLSILGAILWVNRPWRTPWTES

SEQ ID NO: 44 - DNA, 1508 bp, Homo sapiens
  CDS (340)..(1014)
cggacgcgtgggcggacgcgtgggtgaagccagaagctgtgggctgtggggaagtggctgcatcgccacccaagggaccctggactttgccagacgtcagggttgagaggagccactagcaccatcccagagctgtcagcaccggcctcagcccaggcggctctctccctgagcttcctgtaccctgaccctctccagcctcagacctgagacagggctggacaaggaagcagagagcagaagaaaagcagaagcgaagctcagatctgctgggaggagactacattttgtcccctcctggggtcttgcacagtggcaggtgacattcgtgttacaggaatgactgccagggcctgggcctcgtggcggtcttcagctctgctcctcctgcttgtcccaggctattttcctctgagccaccccatgaccgtggcgggccccgtggggggatccctgagtgtgcagtgtcgctatgagaaggaacacaggaccctcaacaaattctggtgcagaccaccacagattctccgatgtgacaagattgtggagaccaaagggtcagcagggaaaaggaatggccgagtgtccatcagggacagtcctgcaaacctcagcttcacagtgaccctggagaatctcacagaggaggacgcaggcacctactggtgtggggtggatacaccgtggctccgagactttcatgatcccattgtcgaggttgaggtgtccgtgttcccggccgggacgaccacagcctccagcccccagagctccatgggcacctcaggtcctcccacgaagctgcccgtgcacacctggcccagcgtgaccagaaaggacagccccgaacccagcccacaccctggctccctgttcagcaatgtccgcttcctgctcctggtcctcttggagctgcccctgctcctgagcatgctgggtgccgtcctctgggtgaacagacctcagagaagctctagaagcaggcagaattggcccaagggtgagaaccagtagcatctgctgtccatcaaggccctgtgctgcaacagagcccctctgggggactggaatgacctcctgaccactccctcccgggctgctctctccacatctcctggaatcctttgtgagcctccttcagccttttccctgtgcccgatcctcatgtgtcacatgtgaacctgacggacatggacgccctgagctgtgagtccacgtctcatgtgcacgccccggccagctaagcctcgggccgtcagcaatacctctgagtgcccggatgccctccctgcacccttccatttctccaggagcctggcactgcttcctccaccgttttcacagggagaagcaggtctaggactcccctgcacctttgccaccataatggcccacactccctgggtccaggagtgtagaatatgcggctgcttccatttgctgagtgtgtcgattcaatgatgctttcaaactttaataaatgccaatgtgtaggttaaaaaaaaaaa

SEQ ID NO: 45 - protein, 224 aa, Homo sapiens
MTARAWASWRSSALLLLLVPGYFPLSHPMTVAGPVGGSLSVQCRYEKEHRTLNKFWCRPPQILRCDKIVETKGSAGKRNGRVSIRDSPANLSFTVTLENLTEEDAGTYWCGVDTPWLRDFHDPIVEVEVSVFPAGTTTASSPQSSMGTSGPPTKLPVHTWPSVTRKDSPEPSPHPGSLFSNVRFLLLVLLELPLLLSMLGAVLWVNRPQRSSRSRQNWPKGENQ

SEQ ID NO: 46 - DNA, 2299 bp, Homo sapiens
  CDS (120)..(725)
cggacgcgtggggcagaaggtgcaagccagagctcaggcagaacttccagagtgcatctgggatctgcatttgccactggttgcagatcaggcggacgaggagccgggaaggcagagccatgtggctgccccctgctctgctccttctcagcctctcaggctgtttctccatccaaggcccagagtctgtgagagccccagagcaggggtccctgacggttcaatgccactataagcaaggatgggagacctacattaagtggtggtgccgaggggtgcgctgggatacatgcaagatcctcattgaaaccagagggtcggagcaaggagagaagagtgaccgtgtgtccatcaaggacaatcagaaagaccgcacgttcactgtgaccatggaggggctcaggcgagatgacgcagatgtttactggtgtgggattgaaagaagaggacctgaccttgggactcaagtgaaagtgatcgttgacccagagggagcggcttccacaacagcaagctcacctaccaacagcaatatggcagtgttcatcggctcccacaagaggaaccactacatgctcctggtatttgtgaaggtgcccatcttgctcatcttggtcactgccatcctctggttgaaggggtctcagagggtccctgaggagccaggggaacagcctatctacatgaacttctccgaacctctgactaaagacatggccacttagagagatggatctgcagagccttcctgccctggccacgtttccagaagagactcgggctgtggaaggaacatctacgagtcctcgggatgcagtgactgagataggggccctgggcctccgccctggccttggagctggtgggcacctccctgttctgcacagctcagggacttagccaggtcctctcctgagccaccatcacctcctggggtgccagcacctgttctcttggtcaggagctgtagagatggagctcaagcactggacgactctgtccccactgctggaataactcgggcacagagcatgggaccaaagtacagaaagaggttgggggagacccccccagccctagacttccatcattccggagaccaactcaacaccgtctttgcctgagaacctgatatatccgtgtttttaaatttttttttttctagcaaagttgggttttaatgacttatgttcataggaaacctctctgatcccacacacaaggagggtgattctgggatgagttcctggttctagggcatgaggggctggatggaccctgtccccagggaggacatggctctgagtccacagggctgaggaggcaatgggaacctccctggcccggcccggtgcttgtcctccccctcccacctcttcctcctcctagctccccaagctccctgcctattcccccacctccgaggggctgcagcttgggagcctcctcagcatgacagcttgggtctcctccccaaaagagcctgtcaggcctcaagaaccacctccaggtggggagggcagtaacgaaaaccatcgcaggaaatggcaccctcccttttcggtgatgttgaaatcatgttactaatgaaaactgtcctagggaagtggttctgtctcctcacaggcttcacccacggcgatgaggcccttgaatgtggtcactttgtgctgtatggttgagggaccctcacaccaaagggaccttcccatgtgagatgtgctcccgcccccacctgcccacaagcaaacacaccacacatgttcggcatgttgccgtttgaacacccatgaggacgcctccaacctgctcttggttctaatagggagtactgactgtcagcagtggataaaggagaggggaccctctggtccctagcatggcacccagagcctcccctcttcttgtccttcagccaaagagaaactttctctgactttgaactgaatttaggtctctggccaatgatgggcctgaaaattccataatggccagagaggagagttcgagcccggctaagatcccctgagtcattctgtgagggaccaagacccacagtccaccagccccagggccctacctcctggaatgctttcctggatccagcttcccgaagatccgaccagacccagggaggacggcaccgctccgcgggagggaaagccaaagcatggtgcttcaccagctggactcaggggcgaggggacatgggcgcttgtcaacgtgatgtcattcttttcccaccgtttcttcctgttgatattcaatgaatccgtcaatctctctggaaaaaaaaaaa

SEQ ID NO: 47 - protein, 201 aa, Homo sapiens
MWLPPALLLLSLSGCFSIQGPESVRAPEQGSLTVQCHYKQGWETYIKWWCRGVRWDTCKILIETRGSEQGEKSDRVSIKDNQKDRTFTVTMEGLRRDDADVYWCGIERRGPDLGTQVKVIVDPEGAASTTASSPTNSNMAVFIGSHKRNHYMLLVFVKVPILLILVTAILWLKGSQRVPEEPGEQPIYMNFSEPLTKDMAT

SEQ ID NO: 48 - DNA, 1847 bp, Homo sapiens
  CDS (303)..(1202)
gacccacgcgtccggctacggagtcactacagggagaggtctcatcactagaaatagccgaagaacctgcagcctcaaccagcgttaaggaggacgtgccgtttctagggagtcttagtcggggctcaccagcttcctgcattttctgaccggctccaggaatagaaccaaaagaagctgaacaaggaagcagcgcggcaccaagaaaagcagaagtcggggccttggaggcgtgactttcccctcgggtccaggtagggcctggagctgctgcaagtgccgcctgtgctggggaagggaccatgtggctgccttgggctctgttgcttctctgggtcccaggatgttttgctctgagcaaatgcaggaccgtggcgggccccgtggggggatccctgagtgtgcagtgtccctatgagaaggaacacaggaccctcaacaaatactggtgcagaccaccacagattttcctatgtgacaagattgtggagaccaaagggtcagcaggaaaaaggaacggccgagtgtccatcagggacagtcctgcaaacctcagcttcacagtgaccctggagaatctcacagaggaggatgcaggcacctactggtgtggggtggatacaccgtggctccgagactttcatgatcccgttgtcgaggttgaggtgtccgtgttcccggcatcaacgtcaatgacacctgcaagtatcactgcggccaagacctcaacaatcacaactgcatttccacctgtatcatccactaccctgtttgcagtgggtgccacccacagtgccagcatccaggaggaaactgaggaggtggtgaactcacagctcccgctgctcctctccctgctggcattgttgctgcttctgttggtgggggcctccctgctagcctggaggatgtttcagaaatggatcaaagctggtgaccattcagagctgtcccagaaccccaagcaggctgccacgcagagtgagctgcactacgcaaatctggagctgctgatgtggcctctgcaggaaaagccagcaccaccaagggaggtggaggtggaatacagcactgtggcctcccccagggaagaacttcactatgcctcggtggtgtttgattctaacaccaacaggatagctgctcagaggcctcgggaggaggaaccagattcagattacagtgtgataaggaagacataggcttttgtcctgcctcgccatcggagctctcatgggccccaggaagtccagggacagctcccttatacctggcccacgtccttctcagcctgccctcgacaacagtgaccaacagacaggcagctgggtttcccaggccatccctctgttgccatcagcttgattggcttccccgagggccagcagggctgggggctccggagagcagcaggaagcactcccagccaccagtgcctgtcgcctctttcccctttgcccctgcttcatcccagctctgtgtgtggaggacaaagcttcttcctgcgtggctccaggaaaagatgtggctcacgtaggtggcacctgccaatagctttgtcaatcacagccccataggaacgtctggaattgcttgggagttggggagaactgtcaagaagagtgaagagagtgccaaagcggagatctgttcacctgggggccatggaggggggacccactaaagatcaagatcaaagattctccccatctcacagacaaggaaactgaggccagagggaggagagaattgctcatggctccagaactggtggcaagtttctctggactcttaggtttatttttaatatgaaatataaaaacagtttcaaatatcttaaaaaaaaa

SEQ ID NO: 49 - protein, 299 aa, Homo sapiens
MWLPWALLLLWVPGCFALSKCRTVAGPVGGSLSVQCPYEKEHRTLNKYWCRPPQIFLCDKIVETKGSAGKRNGRVSIRDSPANLSFTVTLENLTEEDAGTYWCGVDTPWLRDFHDPVVEVEVSVFPASTSMTPASITAAKTSTITTAFPPVSSTTLFAVGATHSASIQEETEEVVNSQLPLLLSLLALLLLLLVGASLLAWRMFQKWIKAGDHSELSQNPKQAATQSELHYANLELLMWPLQEKPAPPREVEVEYSTVASPREELHYASVVFDSNTNRIAAQRPREEEPDSDYSVIRKT

SEQ ID NO: 50 - DNA, 2788 bp, Homo sapiens
  CDS (69)..(1067)
tctggcgaggtgacagcgtagaaccagacagttcccagcgtctgctcccacggtgtccagcgcccagaatgcggcttctggtcctgctatggggttgcctgctgctcccaggttatgaagccctggagggcccagaggaaatcagcgggttcgaaggggacactgtgtccctgcagtgcacctacagggaagagctgagggaccaccggaagtactggtgcaggaagggtgggatcctcttctctcgctgctctggcaccatctatgcagaagaagaaggccaggagacaatgaagggcagggtgtccatccgtgacagccgccaggagctctcgctcattgtgaccctgtggaacctcaccctgcaagacgctggggagtactggtgtggggtcgaaaaacggggccccgatgagtctttactgatctctctgttcgtctttccaggaccctgctgtcctccctccccttctcccaccttccagcctctggctacaacacgcctgcagcccaaggcaaaagctcagcaaacccagcccccaggattgacttctcctgggctctacccggcagccaccacagccaagcaggggaagacaggggctgaggcccctccattgccagggacttcccagtacgggcacgaaaggacttctcagtacacaggaacctctcctcacccagcgacctctcctcctgcagggagctcccgcccccccatgcagctggactccacctcagcagaggacaccagtccagctctcagcagtggcagctctaagcccagggtgtccatcctgatggtccgcatactggccccagtcctggtgctgctgagccttctgtcagccgcaggcctgatcgccttctgcagccacctgctcctgtggagaaaggaagctcaacaggccacggagacacagaggaacgagaagttctgcctctcacgcttgactgcggaggaaaaggaagccccttcccaggcccctgagggggacgtgatctcgatgcctcccctccacacatctgaggaggagctgggcttctcgaagtttgtctcagcgtagggcaggaggccctcctggccaggccagcagtgaagcagtatggctggctggatcagcaccgattcccgaaagctttccacctcagcctcagagtccagctgcccggactccagggctctccccaccctccccaggctctcctcttgcatgttccagcctgacctagaagcgtttgtcagccctggagcccagagcggtggccttgctcttccggctggagactgggacatccctgataggttcacatccctgggcagagtaccaggctgctgaccctcagcagggccagacaaggctcagtggatctggtctgagtttcaatctgccaggaactcctgggcctcatgcccagtgtcggaccctgccttcctcccactccagaccccaccttgtcttccctccctggcgtcctcagacttagtcccacggtctcctgcatcagctggtgatgaagaggagcatgctggggtgagactgggattctggcttctctttgaaccacctgcatcccagcccttcaggaagcctgtgaaaaacgtgattcctgggccccaccaagacccaccaaaaccatctctggggcttggtgcaggactctgaatttctaacaatgcccagtgactgtcgcacttgagtttgagggccagtgggcctgatgaacgctcagacccctccagcttagagtctgcatttgggctgtgacgtctcccacctgccccaataagatctgctctgtctgcgacaccaagatccagctggggactcccctgaggcctgcctaagtccaggccttggtcaggtcaggtgcacattgcagggataagcccaggaccggcagagagtggttgcctttccatttgccctccctggccatgccttcttgcctttggaaaaatgatgaagaaaaccttggctccttccttgtctggaaagggttacttgcctatgggttctggtggctagagagaaaagtagaaaaccagagtgcacgtaggtgtctaacacagaggagagtaggaacagggcggatacctgaaggtgactccgagtccagccccctggagaaggggtcgggggtggtggtaaagtagcacaactactattttttttctttttccattattattgttttttaagacagaatctcgtgctgctgcccaggctggagtgcagtggcacgatctgcaaactccgcctcctgggttcaagtgattcttctgcctcagcctcccgagtagctgggattacaggcacgcaccaccacacctggctaatttttgtacttttagtagagatggggtttcaccatgttggccaggctggtcttgaactcctgacctcaaatgagcctcctgcttcagtctcccaaattgccgggattacaggcatgagccactgtgtctggccctatttcctttaaaaagtgaaattaagagttgttcagtatgcaaaacttggaaagatggaggagaaaaagaaaaggaagaaaaaaatgtcacccatagtctcaccagagactatcattatttcgttttgttgtacttcctttccactcttttcttcttcacataatttgccggtgttctttttacagagcaattatcttgtatatacaactttgtatcctgccttttccaccttatcgttccatcactttattccagcacttctctgtgttttacagacctttttataaataaaatgttcatcagctgcaaaaa

SEQ ID NO: 51 - protein, 332 aa, Homo sapiens
MRLLVLLWGCLLLPGYEALEGPEEISGFEGDTVSLQCTYREELRDHRKYWCRKGGILFSRCSGTIYAEEEGQETMKGRVSIRDSRQELSLIVTLWNLTLQDAGEYWCGVEKRGPDESLLISLFVFPGPCCPPSPSPTFQPLATTRLQPKAKAQQTQPPGLTSPGLYPAATTAKQGKTGAEAPPLPGTSQYGHERTSQYTGTSPHPATSPPAGSSRPPMQLDSTSAEDTSPALSSGSSKPRVSILMVRILAPVLVLLSLLSAAGLIAFCSHLLLWRKEAQQATETQRNEKFCLSRLTAEEKEAPSQAPEGDVISMPPLHTSEEELGFSKFVSA

SEQ ID NO: 52 - DNA, 423 bp, Homo sapiens
attgtcactcaaatcaccggtccaacaacagtgaatggcttggagcggggctccttgaccgtgcagtgtgtttacagatcaggctgggagacctacttgaagtggtggtgtcgaggagctatttggcgtgactgcaagatccttgttaaaaccagtgggtcagagcaggaggtgaagagggaccgggtgtccatcaaggacaatcagaaaaaccgcacgttcactgtgaccatggaggatctcatgaaaactgatgctgacacttactggtgtggaattgagaaaactggaaatgaccttggggtcacagttcaagtgaccattgacccagcgtcgactcctgcccccaccacgcctacctccactacgtttacagcaccagtcacccaagaagaaactagcagctccccaactctgaccggc

SEQ ID NO: 53 - DNA, 25 bp, Artificial
  Primer sequence
atgattgtcactcaaatcaccggtc

SEQ ID NO: 54 - DNA, 28 bp, Artificial
  Primer sequence
ttattagccggtcagagttggggagctg

SEQ ID NO: 55 - DNA, 25 bp, Artificial
  Primer sequence
atgattgtcactcaaatcaccggtc

SEQ ID NO: 56 - DNA, 22 bp, Artificial
  Primer sequence
gccggtcagagttggggagctg

SEQ ID NO: 57 - DNA, 915 bp, Homo sapiens
  CDS (1)..(915)
atgcccctgctgacactctacctgctcctcttctggctctcaggctactccattgtcactcaaatcaccggtccaacaacagtgaatggcttggagcggggctccttgaccgtgcagtgtgtttacagatcaggctgggagacctacttgaagtggtggtgtcgaggagctatttggcgtgactgcaagatccttgttaaaaccagtgggtcagagcaggaggtgaagagggaccgggtgtccatcaaggacaatcagaaaaaccgcacgttcactgtgaccatggaggatctcatgaaaactgatgctgacacttactggtgtggaattgagaaaactggaaatgaccttggggtcacagttcaagtgaccattgacccagcgtcgactcctgcccccaccacgcctacctccactacgtttacagcaccagtcacccaagaagaaactagcagctccccaactctgaccggccaccacttggacaacaggcacaagctcctgaagctcagtgtcctcctgcccctcatcttcaccatattgctgctgcttttggtggccgcctcactcttggcttggaggatgatgaagtaccagcagaaagcagccgggatgtccccagagcaggtactgcagcccctggagggcgacctctgctatgcagacctgaccctgcagctggccggaacctccccgcgaaaggctaccacgaagctttcctctgcccaggttgaccaggtggaagtggaatatgtcaccatggcttccttgccgaaggaggacatttcctatgcatctctgaccttgggtgctgaggatcaggaaccgacctactgcaacatgggccacctcagtagccacctccccggcaggggccctgaggagcccacggaatacagcaccatcagcaggcct

SEQ ID NO: 58 - protein, 305 aa, Homo sapiens; identical to SEQ ID NO: 3

(19) United States
(12) Patent Application Publication
(10) Pub. No.: US 2007/0264261 A1
(43) Pub. Date: Nov. 15, 2007

(54) Methods of Therapy and Diagnosis Using Targeting of Cells that Express Killer Cell Immunoglobulin like Receptor like Proteins

(75) Inventors: Peter C.R. Emtage, Sunnyvale, CA (US); Y. Tom Tang, San Jose, CA (US)

Correspondence Address: NUVELO, INC, 201 INDUSTRIAL ROAD, SUITE 310, SAN CARLOS, CA, 94070, US

(73) Assignee: NUVELO, INC., San Carlos, CA (US)

(21) Appl. No.: 11/766,911

(22) Filed: Jun. 22, 2007

Related U.S. Application Data
(62) Division of application No. 10/962,127, filed on Oct. 8, 2004.
Application No. 10/962,127 is a continuation-in-part of application No. PCT/US04/11171, filed on Apr. 13, 2004.
Application No. PCT/US04/11171 is a continuation-in-part of application No. 10/727,012, filed on Dec. 2, 2003, now abandoned.
Application No. 10/727,012 is a continuation-in-part of application No. 10/414,539, filed on Apr. 14, 2003, now abandoned.

(51) Int. Cl. A61K 39/395; A61K 38/00; A61K 48/00; C07H 21/04; C07K 14/00; C07K 16/18; C12N 15/00; C12N 5/06; C12Q 1/68; G01N 33/53

(52) U.S. Cl. 424/138.1; 435/320.1; 435/325; 435/6; 435/7.23; 514/2; 514/44; 530/350; 530/387.1; 530/388.1; 536/23.1

(57) ABSTRACT

Certain cells, including various types of cancer cells, express KIRHy proteins. Targeting using KIRHy polypeptides, nucleic acids encoding for KIRHy polypeptides and anti-KIRHy antibodies provides a method of killing or inhibiting that growth of cancer cells that express the KIRHy protein. Methods of therapy and diagnosis of disorders associated with KIRHy protein-expressing cells, such as acute myelogenous leukemia (AML), are described.

### 1. CROSS REFERENCE TO RELATED APPLICATIONS

This application is a divisional application of U.S. application Ser. No. 10/962,127 filed on Oct. 8, 2004, which in turn is a continuation-in-part application of PCT Application Serial No. PCT/US04/11171 filed on Apr. 13, 2004, which in turn is a continuation-in-part application of U.S. application Ser. No. 10/727,012 filed on Dec. 2, 2003, which in turn is a continuation-in-part application of U.S. application Ser. No. 10/414,539 filed on Apr. 14, 2003. These and all other U.S. Patents and Patent Applications cited herein are hereby incorporated by reference in their entirety.

### 2. BACKGROUND

2.1 Technical Field

This invention relates to compositions and methods for targeting killer cell immunoglobulin-like receptor-like protein (herein denoted KIRHy)-expressing cells using antibodies, polypeptides, polynucleotides, peptides, and small molecules and their use in the therapy and diagnosis of various pathological states, including cancer, such as acute myelogenous leukemia (AML).

2.2 Sequence Listing

The sequences of the polynucleotide and polypeptide of the invention are listed in the sequence listing and are submitted on a compact disc containing the file labeled “NUVO-02CP3DIV.txt”—136 KB (139,489 bytes), which was created on an IBM PC, Windows 2000 operating system on Jun. 20, 2007 at 3:05:00 PM. The sequence listing entitled “NUVO-02CP3DIV.txt” is herein incorporated by reference in its entirety.

2.3 Background Art

Immunotherapy provides a method of harnessing the immune system to treat various pathological states, including cancer, autoimmune disease, transplant rejection, hyperproliferative conditions, allergic reactions, emphysema, and wound healing.

For example, antibody therapy for cancer involves the use of antibodies, or antibody fragments, against a tumor antigen to target antigen-expressing cells. Antibodies, or antibody fragments, may have direct or indirect cytotoxic effects or may be conjugated or fused to cytotoxic moieties. Direct effects include the induction of apoptosis, the blocking of growth factor receptors, and anti-idiotype antibody formation. Indirect effects include antibody-dependent cell-mediated cytotoxicity (ADCC) and complement-mediated cellular cytotoxicity (CMCC). When conjugated or fused to cytotoxic moieties, the antibodies, or fragments thereof, provide a method of targeting the cytotoxicity towards the tumor antigen expressing cells. (Green, et al., Cancer Treatment Reviews, 26:269-286 (2000), incorporated herein by reference in its entirety).

Because antibody therapy targets cells expressing a particular antigen, there is a possibility of cross-reactivity with normal cells or tissue. Although some cells, such as hematopoietic cells, are readily replaced by precursors, cross-reactivity with many tissues can lead to detrimental results. Thus, considerable research has gone towards finding tumor-specific antigens. Such antigens are found almost exclusively on tumors or are expressed at a greater level in tumor cells than the corresponding normal tissue. Tumor-specific antigens provide targets for antibody targeting of cancer, or other disease-related cells, expressing the antigen. Antibodies specific to such tumor-specific antigens can be conjugated to cytotoxic compounds or can be used alone in immunotherapy. Immunotoxins target cytotoxic compounds to induce cell death. For example, anti-CD22 antibodies conjugated to deglycosylated ricin A may be used for treatment of B cell lymphoma that has relapsed after conventional therapy (Amlot, et al., Blood 82:2624-2633 (1993), incorporated herein by reference in its entirety) and has demonstrated encouraging responses in initial clinical studies.

The immune system functions to eliminate organisms or cells that are recognized as non-self, including microorganisms, neoplasms and transplants. A cell-mediated host response to tumors includes the concept of immunologic surveillance, by which cellular mechanisms associated with cell-mediated immunity, destroy newly transformed tumor cells after recognizing tumor-associated antigens (antigens associated with tumor cells that are not apparent on normal cells). Furthermore, a humoral response to tumor-associated antigens enables destruction of tumor cells through immunological processes triggered by the binding of an antibody to the surface of a cell, such as antibody-dependent cellular cytotoxicity (ADCC) and complement mediated lysis.

Recognition of an antigen by the immune system triggers a cascade of events including cytokine production, B-cell proliferation, and subsequent antibody production. Often tumor cells have reduced capability of presenting antigen to effector cells, thus impeding the immune response against a tumor-specific antigen. In some instances, the tumor-specific antigen may not be recognized as non-self by the immune system, preventing an immune response against the tumor-specific antigen from occurring. In such instances, stimulation or manipulation of the immune system provides effective techniques of treating cancers expressing one or more tumor-specific antigens.

For example, Rituximab (RITUXAN®) is a chimeric antibody directed against CD20, a B cell-specific surface molecule found on >95% of B-cell non-Hodgkin's lymphoma (Press, et al., Blood 69:584-591 (1987); Malony, et al., Blood 90:2188-2195 (1997), both of which are incorporated herein in their entirety). Rituximab induces ADCC and inhibits cell proliferation through apoptosis in malignant B cells in vitro (Maloney, et al., Blood 88:637a (1996), incorporated herein by reference in its entirety). Rituximab is currently used as a therapy for advanced stage or relapsed low-grade non-Hodgkin's lymphoma, which has not responded to conventional therapy.

Active immunotherapy, whereby the host is induced to initiate an immune response against its own tumor cells can be achieved using therapeutic vaccines. One type of tumor-specific vaccine uses purified idiotype protein isolated from tumor cells, coupled to keyhole limpet hemocyanin (KLH) and mixed with adjuvant for injection into patients with low-grade follicular lymphoma (Hsu, et al., Blood 89:3129-3135 (1997), incorporated herein by reference in its entirety). Another type of vaccine uses antigen-presenting cells (APCs), which present antigen to naïve T cells during the recognition and effector phases of the immune response. Dendritic cells, one type of APC, can be used in a cellular vaccine in which the dendritic cells are isolated from the patient, co-cultured with tumor antigen and then reinfused as a cellular vaccine (Hsu, et al., Nat. Med. 2:52-58 (1996), incorporated herein by reference in its entirety). Immune responses can also be induced by injection of naked DNA. Plasmid DNA that expresses bicistronic mRNA encoding both the light and heavy chains of tumor idiotype proteins, such as those from B cell lymphoma, when injected into mice, are able to generate a protective, anti-tumor response (Singh, et al., Vaccine 20:1400-1411 (2002)).

Myelodysplastic and myeloproliferative diseases are diseases of the bone marrow. Myeloproliferative disorders are diseases in which too many of certain types of blood cells are made in the bone marrow, such as leukemia, lymphoma, and hypereosinophilic syndrome. Myelodysplastic syndromes, also called “pre-leukemia” or “smoldering” leukemia, are disorders in which the bone marrow does not function normally and not enough blood cells are made. Myeloproliferative diseases account for over 200,000 patients in the U.S. alone. Acute myelogenous leukemia (AML) is a myeloproliferative disease in which there is a cancerous overgrowth of immature blood cells within the bone marrow and blood. This leads to impairment in the production of normal blood components, specifically red cells, white cells and platelets.

About 10,600 new cases of acute myelogenous leukemia (AML) are diagnosed each year in the U.S. AML may be called by several names including acute myelocytic leukemia, acute myeloblastic leukemia, acute granulocytic leukemia, and acute non-lymphocytic leukemia. AML is the most common leukemia diagnosed in adults and accounts for just under half of the cases of childhood leukemia. AML is frequently sub-classified into eight well-defined variants, M0 through M7, all of which, with the exception of M3, are initially treated the same way. The most important feature of each individual's AML is the presence and nature of the chromosomal abnormalities within the leukemia cells, some of which are known to predict a better outcome. Although leukemia starts in the bone marrow, it can spread to the blood, lymph nodes, spleen, liver, central nervous system, and other organs. It does not usually form a solid mass or tumor. Chemotherapy and blood stem cell transplantation are the primary forms of treatment and result in cure in many patients. However, recurrence following treatment is a major problem and is often the cause of death. While AML affects people of many ages (the median age is 64 years), the prognosis is poor in older individuals. In addition, the toxic effects of therapeutic outcomes and the presence of drug refractoriness remain considerable problems that need to be overcome to improve the quality of life and reduce the death rate of cancer patients. Graft-vs-host-disease (GVHD) is a major problem of allogenic blood stem cell transplants that come from a donor, whereas relapse due to latent AML cells is a problem in autologous blood stem cell transplants that come from blood stem cells isolated from the patient before undergoing chemotherapy.

Despite the advances in therapy, mortality remains high. Currently, therapy consists of either induction therapy comprising an anthracycline (i.e. daunorubicin) or anthraquinone with or without cytarabine, or hematopoetic cell transplants. The deployment of immunotherapy as a treatment option against AML and other cancers remains hampered by the lack of tumor-associated antigens that are tumor-specific, strongly immunogenic and that are shared among different patients (Dalerba et al., Clin. Rev. Oncol. Hematol. 46:33-57 (2003)). Therefore, there exists a need in the art to identify antigens that are clearly and specifically expressed on the surface of cancer cells that could serve as targets for various targeting strategies. The present invention identifies a family of molecular targets useful for therapeutic intervention in AML and other myeloproliferative and myelodysplastic disorders and provides herein methods for the diagnosis and therapy of myeloproliferative disorders.

### 3. SUMMARY OF THE INVENTION

The invention provides therapeutic and diagnostic methods of targeting cells expressing killer cell immunoglobulin-like receptor (KIR)-like protein 1 (herein denoted as KIRHy1), its variants, KIRHy2-8, and human homologs KIRL1-7 (for KIRHy-like proteins). For the sake of convenience, KIRHy1, its variants and homologs are herein referred to generally as KIRHy. The individual gene names will be used when describing a specific KIRHy molecule. The therapeutic and diagnostic methods of the invention utilize targeting elements such as KIRHy polypeptides, nucleic acids encoding KIRHy proteins, and anti-KIRHy antibodies, including fragments or other modifications thereof, peptides and small molecules. KIRHy proteins are highly expressed in certain hematopoietic-based cancer cells relative to its expression in healthy cells, thus, targeting cells that express KIRHy will have a minimal effect on healthy tissues while destroying or inhibiting the growth of the hematopoietic-based cancer cells. Similarly, non-hematopoietic type tumors (solid tumors) can be targeted if they bear a KIRHy antigen. For example, inhibition of growth and/or destruction of KIRHy-expressing cancer cells results from targeting such cells with anti-KIRHy antibodies. One embodiment of the invention comprises a method of destroying KIRHy-expressing cells by conjugating anti-KIRHy antibodies with cytocidal materials such as radioisotopes or other cytotoxic compounds.

The present invention provides a variety of targeting elements and compositions. One such embodiment is a composition comprising an anti-KIRHy antibody preparation. Exemplary antibodies include a single anti-KIRHy antibody, a combination of two or more anti-KIRHy antibodies, a combination of an anti-KIRHy antibody with a non-KIRHy antibody, a combination of an anti-KIRHy antibody and a therapeutic agent, a combination of an anti-KIRHy antibody and a cytocidal agent, a bispecific anti-KIRHy antibody, Fab KIRHy antibodies or fragments thereof, including any fragment of an antibody that retains one or more complementarity-determining regions (CDRs) that recognize KIRHy, humanized anti-KIRHy antibodies that retain all or a portion of a CDR that recognizes KIRHy, anti-KIRHy conjugates, and anti-KIRHy antibody fusion proteins.

Another targeting embodiment of the invention is a composition comprising a KIRHy antigen, for example, a KIRHy polypeptide, or fragment thereof, and optionally comprising a suitable adjuvant.

Yet another targeting embodiment is a composition comprising a nucleic acid encoding a KIRHy polypeptide, or a fragment or variant thereof, optionally within a recombinant vector. A further targeting embodiment of the present invention is a composition comprising an antigen-presenting cell transformed with a nucleic acid encoding a KIRHy polypeptide, or a fragment or variant thereof, optionally within a recombinant vector.

Yet another targeting embodiment of the invention is a preparation comprising a KIRHy polypeptide or peptide fragment thereof. A further targeting embodiment of the present invention is a non-KIRHy polypeptide or peptide that binds a KIRHy polypeptide or polynucleotide of the invention.

Another targeting embodiment of the invention is a preparation comprising a small molecule that recognizes or binds to a KIRHy polypeptide or polynucleotide of the invention.

The present invention further provides a method of targeting KIRHy-expressing cells, which comprises administering a targeting element or composition in an amount effective to target KIRHy-expressing cells. Any one of the targeting elements or compositions described herein may be used in such methods, including an anti-KIRHy antibody preparation, a KIRHy antigen comprising a KIRHy polypeptide, or a fragment thereof, a composition of a nucleic acid encoding a KIRHy polypeptide, or a fragment or variant thereof, optionally within a recombinant vector, or a composition of an antigen-presenting cell transformed with a nucleic acid encoding a KIRHy polypeptide, or fragment or variant thereof, optionally within a recombinant vector.

The present invention further provides a method of targeting KIRHy-expressing cells, which comprises administering a targeting element or composition in an amount effective to target KIRHy-expressing cells. Any one of the targeting elements or compositions described herein may be used in such methods, including an anti-KIRHy antibody preparation, a KIRHy antigen comprising a KIRHy polypeptide, or a fragment or variant thereof, a composition of a nucleic acid encoding a KIRHy polypeptide, or a fragment or variant thereof, optionally with a recombinant vector, a composition of an antigen-presenting cell transformed with a nucleic acid encoding a KIRHy polypeptide, or fragment thereof or variant, optionally within a recombinant vector, a KIRHy polypeptide, peptide fragment thereof, or a binding polypeptide, peptide or small molecule that binds to a KIRHy polypeptide or polynucleotide of the invention.

The invention also provides a method of inhibiting the growth of cancer cells, including hematopoietic-based cancer cells and KIRHy-expressing cancer cells, which comprises administering a targeting element or a targeting composition in an amount effective to inhibit the growth of said cancer cells. Any one of the targeting elements or compositions described herein may be used in such methods, including an anti-KIRHy antibody preparation, a KIRHy antigen comprising a KIRHy polypeptide, or fragment thereof, a composition of a nucleic acid encoding a KIRHy polypeptide, or fragment or variant thereof, optionally within a recombinant vector, a composition of an antigen-presenting cell transformed with a nucleic acid encoding a KIRHy polypeptide, or fragment or variant thereof, optionally within a recombinant vector, a KIRHy polypeptide, peptide fragment thereof, or a binding polypeptide, peptide or small molecule that binds to a KIRHy polypeptide or polynucleotide of the invention.

The present invention further provides a method of treating disorders associated with the proliferation of KIRHy-expressing cells in a subject in need thereof, comprising the step of administering a targeting element or targeting composition in a therapeutically effective amount to treat disorders associated with KIRHy-expressing cells. Any one of the targeting elements or compositions described herein may be used in such methods, including an anti-KIRHy antibody preparation, a KIRHy antigen comprising a KIRHy polypeptide, fragment thereof, a composition of a nucleic acid encoding a KIRHy polypeptide, or fragment or variant thereof, optionally within a recombinant vector, or a composition of an antigen-presenting cell comprising a nucleic acid encoding KIRHy, or fragment or variant thereof, optionally within a recombinant vector, or a KIRHy polypeptide, peptide fragment thereof, or a binding polypeptide, peptide or small molecule that binds to or recognizes a KIRHy polypeptide or polynucleotide of the invention.

Examples of disorders associated with the proliferation of KIRHy-expressing cells include cancers, such as acute myelogenous leukemia (AML) and histiocytic lymphoma. In addition, other KIRHy-expressing cells can include diseases such as Hodgkin's Disease, non-Hodgkin's B-cell lymphomas, T-cell lymphomas, malignant lymphoma, lymphosarcoma leukemia, chronic lymphocytic leukemia, multiple myeloma, chronic myeloid leukemia (also known as myelogenous leukemia), chronic myelomonocytic leukemia, myelodysplastic syndromes, myeloproliferative disorders, hypereosinophilic syndrome, eosinophilic leukemia, multiple myeloma, X-linked lymphoproliferative disorders; Epstein Barr Virus-related conditions such as mononucleosis; hyperproliferative disorders; autoimmune disorders; wound healing; and organ and tissue transplantation rejection (including hyperacute, acute, chronic and xenograft transplant rejection). Non-hematopoietic tumors that bear a KIRHy antigen, such as esophageal cancer, stomach cancer, colon cancer, colorectal cancer, polyps associated with colorectal neoplasms, pancreatic cancer and gallbladder cancer, cancer of the adrenal cortex, ACTH-producing tumor, bladder cancer, brain cancer including intrinsic brain tumors, neuroblastomas, astrocytic brain tumors, gliomas, and metastatic tumor cell invasion of the central nervous system, Ewing's sarcoma, head and neck cancer including mouth cancer and larynx cancer, kidney cancer including renal cell carcinoma, liver cancer, lung cancer including small and non-small cell lung cancers, malignant peritoneal effusion, malignant pleural effusion, skin cancers including malignant melanoma, tumor progression of human skin keratinocytes, epithelial cell carcinoma, squamous cell carcinoma, basal cell carcinoma, and hemangiopericytoma, mesothelioma, Kaposi's sarcoma, bone cancer including osteomas and sarcomas such as fibrosarcoma and osteosarcoma, cancers of the female reproductive tract including uterine cancer, endometrial cancer, ovarian cancer, ovarian (germ cell) cancer and solid tumors in the ovarian follicle, vaginal cancer, cancer of the vulva, and cervical cancer; breast cancer (small cell and ductal), penile cancer, prostate cancer, retinoblastoma, testicular cancer, thyroid cancer, trophoblastic neoplasms, and Wilms' tumor, can also be targeted. The invention further provides a method of modulating the immune system by either suppression or stimulation of growth factors and cytokines, by administering the targeting elements or compositions of the invention. The invention also provides a method of modulating the immune system through activation of immune cells (such as natural killer cells, T cells, B cells and myeloid cells), through the suppression of activation, or by stimulating or suppressing proliferation of these cells by KIRHy peptide fragments or KIRHy antibodies.

The present invention further provides a method of treating immune-related disorders by suppressing the immune system in a subject in need thereof, by administering the targeting elements or compositions of the invention. Such immune-related disorders include but are not limited to autoimmune disease and organ transplant rejection.

The present invention also provides a method of diagnosing disorders associated with KIRHy-expressing cells comprising the step of measuring the expression patterns of KIRHy protein and/or its associated mRNA. Yet another embodiment of the invention provides a method of diagnosing disorders associated with KIRHy-expressing cells comprising the step of detecting KIRHy expression using anti-KIRHy antibodies. Expression levels or patterns may then be compared with a suitable standard indicative of the desired diagnosis. Such methods of diagnosis include compositions, kits and other approaches for determining whether a patient is a candidate for KIRHy therapy in which said KIRHy is targeted.

The present invention also provides a method of enhancing the effects of therapeutic agents and adjunctive agents used to treat and manage disorders associated with KIRHy-expressing cells, by administering KIRHy preparations of said KIRHy with therapeutic and adjuvant agents commonly used to treat such disorders.

### 4. BRIEF DESCRIPTION OF THE DRAWING

FIG. 1 depicts a BLASTP amino acid sequence alignment between the protein encoded by SEQ ID NO: 2 (i.e. SEQ ID NO: 3) KIRHy1 and human Natural Killer (NK) inhibitory receptor precursor (SEQ ID NO: 8), indicating that the two sequences share 94% similarity and 94% identity over the entire amino acid sequence of SEQ ID NO: 3.

FIG. 2 depicts a BLASTP amino acid sequence alignment between the protein encoded by SEQ ID NO: 2 (i.e. SEQ ID NO: 3) KIRHy1 and human CMRF35-like protein (similar to CMRF35 leukocyte Ig-like receptor) (SEQ ID NO: 9), indicating that the two sequences share 89% similarity and 89% identity over 149 amino acids of SEQ ID NO: 3.

FIG. 3 depicts a ClustalW multiple sequence alignment of KIRHy1 (SEQ ID NO: 3) and four of its splice variants (KIRHy2, KIRHy3, KIRHy4, and KIRHy5, corresponding to SEQ ID NO: 13, 15, 17, and 19, respectively), wherein asterisks (*) represent identical amino acids, colons (:) represent conservative substitutions, periods (.) represent non-conservative substitutions, and gaps are represented as dashes.

FIG. 4 shows a schematic of the CDS exon mapping of KIRHy1 and its variants, KIRHy2, KIRHy3, KIRHy4, and KIRHy5.

FIG. 5 depicts a ClustalW multiple sequence alignment of KIRHy1 (SEQ ID NO: 3) and three of its splice variants (KIRHy6, KIRHy7, and KIRHy8, corresponding to SEQ ID NO: 21, 23, and 25, respectively), wherein asterisks (*) represent identical amino acids, colons (:) represent conservative substitutions, periods (.) represent non-conservative substitutions, and gaps are represented as dashes.

FIG. 6 shows a schematic of the CDS exon mapping of KIRHy1 and its variants, KIRHy6, KIRHy7, and KIRHy8.

FIG. 7 shows the genomic organization of the CLM gene cluster. In humans, it is localized on Chromosome 17 within the [42.4-73.29] Mb range. In mice, it is localized on Chromosome 11 in the [102.81-116.14] Mb range.

FIG. 8 shows the relative expression of KIRHy1 mRNA (as determined by RT-PCR) derived from healthy tissues, cell lines derived from acute monocytic leukemia (AML193), acute myeloid leukemia (AML565), acute myelogenous leukemia (KG1), anaplastic large T cell lymphoma (L5664), B cell lymphoma (RA1), chronic myelogenous leukemia (K562), diffuse large B cell lymphoma (L22601), follicular lymphoma grades II/III (L5856), histiocytic lymphoma (U937), Hodgkin's lymphoma (HD5664), large B cell lymphoma (DB), non-Hodgkin's lymphoma (RL), and plasmacytoma (RPMI), and tumor tissues derived from B cell lymphoma (H02-85T, H02-86T, H02-87T, H02-88T, H02-89T), follicular lymphoma (H02-74T, H02-75T, H02-76T, H02-77T, H02-78T), and myeloma (H02-79T, H02-80T, H02-81T, H02-82T, H02-83T, H02-84T).

FIG. 9 shows (A) the nucleotide sequence of the KIRHy1-Fc fragment (herein denoted as 10458-Fc) open reading frame (ORF) (SEQ ID NO: 30) comprising a CD33 signal peptide (in italics, nt 1-54), the KIRHy1 extracellular domain (in bold, nt 55-555), and the Fc fragment (in underline, nt 556-1254) and (B) the vector map of the 10458-Fc fragment in the Signal pig Plus vector.

FIG. 10 depicts the sequential deletion of the extracellular domain of KIRHy1 used for epitope mapping of monoclonal antibody (mAb) Clone #20.

FIG. 11 shows FACS histograms illustrating the specificity of Clone #20 for OCI-AML-2 cells: (A) unstained cells; (B) cells treated with secondary antibody only; (C) cells treated with the IgM isotype control; (D) cells treated with complete medium only; and (E) cells treated with the KIRHy1-specific Clone #20 mAb.

FIG. 12 shows FACS histograms illustrating the blocking of Clone #20 staining using a peptide harboring the Clone #20 epitope: (A) unstained cells; (B) cells treated with the IgM isotype control; (C) cells treated with Clone #20 mAb; (D) cells treated with the Clone #20 mAb and 5 μg of irrelevant peptide; and (E) cells treated with the Clone #20 mAb and 5 μg of a peptide containing the Clone #20 epitope.

FIG. 13 shows FACS histograms illustrating the specificity of cell surface labeling of (A) OCI-AML-2 cells, (B) HEK-293 cells; and (C) a confocal micrograph showing the cell surface staining of OCI-AML-2 cells using Clone #20 mAb.

FIG. 14 shows FACS histograms illustrating KIRHy1-mediated cell killing by internalization using the Mab-Zap reagent: (A) cells treated with IgM isotype control+IgSap; (B) cells treated with Clone #20 mAb+IgSap; (C) cells treated with IgM isotype control+Mab-Zap; and (D) cells treated with Clone #20+Mab-Zap.

FIG. 15 shows FACS histograms illustrating KIRHy1 facilitated CDC-mediated killing of OCI-AML-2 cells: (A) treated with medium only; (B) cells treated with the IgM isotype control; (C) cells treated with IgM isotype control+complement; (D) cells treated with complement only; (E) cells treated with Clone #20 mAb; and (F) cells treated with Clone #20 mAb+complement.

FIG. 16 shows FACS histograms illustrating an example of a positive KIRHy1-Fc mAb, clone #75: (A) cells treated with medium only; (B) cells treated with conditioned medium from the hybridoma library; and (C) cells treated with conditioned medium from clone #75.

### 5. DETAILED DESCRIPTION OF THE INVENTION

The present invention relates to methods of targeting KIRHy-expressing cells using targeting elements, such as polypeptides, nucleic acids, antibodies, binding polypeptides, peptides and small molecules, including fragments or other modifications of any of these elements.

The present invention provides a novel approach for diagnosing and treating diseases and disorders associated with said KIRHy. The method comprises administering an effective dose of targeting preparations such as KIRHy antigens, antigen presenting cells, or pharmaceutical compositions comprising the targeting elements, KIRHy polypeptides, nucleic acids encoding KIRHy polypeptides, anti-KIRHy antibodies, or binding polypeptides, peptides and small molecules that bind to KIRHy polypeptides or polynucleotides, described below. Targeting of KIRHy on the cell membranes is expected to inhibit the growth of or destroy such cells. An effective dose will be the amount of such targeting preparations necessary to target the cell surface KIRHy and inhibit the growth of or destroy the KIRHy-expressing cells and/or metastasis.

A further embodiment of the present invention is to enhance the effects of therapeutic agents and adjunctive agents used to treat and manage disorders associated with said KIRHy, by administering targeting preparations that recognize KIRHy with therapeutic and adjuvant agents commonly used to treat such disorders.

Chemotherapeutic agents useful in treating neoplastic disease and antiproliferative agents and drugs used for immunosuppression include alkylating agents, such as nitrogen mustards, alkyl sulfonates, nitrosoureas, triazenes; antimetabolites, such as folic acid analogs, pyrimidine analogs, and purine analogs; natural products, such as vinca alkaloids, epipodophyllotoxins, antibiotics, and enzymes; miscellaneous agents such as polatinum coordination complexes, substituted urea, methyl hydrazine derivatives, and adrenocortical suppressant; and hormones and antagonists, such as adrenocorticosteroids, progestins, estrogens, androgens, and anti-estrogens (Calebresi and Parks, pp. 1240-1306 in, Eds. A. G Goodman, L. S. Goodman, T. W. Rall, and F. Murad, The Pharmacological Basis of Therapeutics, Seventh Edition, MacMillan Publishing Company, New York, (1985), incorporated herein by reference in its entirety).

Adjunctive therapy used in the management of such disorders includes, for example, radiosensitizing agents, coupling of antigen with heterologous proteins, such as globulin or beta-galactosidase, or inclusion of an adjuvant during immunization.

High doses may be required for some therapeutic agents to achieve levels to effectuate the target response, but may often be associated with a greater frequency of dose-related adverse effects. Thus, combined use of the targeting therapeutic methods of the present invention with agents commonly used to treat disorders associated with KIRHy-expression allows the use of relatively lower doses of such agents resulting in a lower frequency of adverse side effects associated with long-term administration of the conventional therapeutic agents. Thus another indication for the targeting therapeutic methods of this invention is to reduce adverse side effects associated with conventional therapy of these disorders.

5.1 Targeting of KIRHy1

Immune system functions are governed by a complex network of cell surface interactions and associated signaling processes. When a cell surface receptor is activated by its ligand, a signal is sent into the cell; depending upon the signal transduction pathway that is engaged, the signal can be inhibitory or activating.

The cytolytic activity of Natural Killer (NK) cells is regulated by a balance between activating signals that initiate cell lysis and inhibitory signals which prevent cytotoxicity. NK cells recognize and kill certain tumor cells, virally-infected cells, MHC class I-disparate normal hematopoietic cells and mediate acute rejection of bone marrow grafts (Salmon-Divon et al., Bull. Math. Biol. 65:199-218 (2003), herein incorporated by reference in its entirety). Target cells are killed when NK cells receive an excess of activation signals. If the target cell expresses cell surface MHC class I antigens for which the NK cell has a specific receptor (i.e. “self” MHC class I antigens), the NK cell is inhibited from killing the target cell. These specific NK cell receptors are of two types: killer cell immunoglobulin (Ig)-like receptors (KIRs) or C-type lectin-like Ly49 receptors. KIRs send a negative signal when engaged by their MHC ligand downregulating NK cell cytotoxicity activity. KIRs have immunoreceptor tyrosine-based inhibitory motifs (ITIM) in the cytoplasmic domain which are phosphorylated by tyrosine kinases. The ITIM motif common to many KIRs has the sequence I/L/VxYxxL/V, wherein “x” represents any amino acid (SEQ ID NO: 10) (Held et al., Curr. Opin. Immunol. 15:233-237 (2003), herein incorporated by reference in its entirety).

Soluble forms of some of these membrane receptors, like FDF03 and CD54, are described and may serve as markers for pathologic conditions (Borges and Cosman Cytokine and Growth Factor Reviews 11:209-217 (2000), herein incorporated by reference in its entirety). Ig Variable domains are utilized to create a specific binding site while Ig Constant domains may serve as more conserved counter receptor binding module. Recently, CMRF-35 and PIGR-1 immunoglobulin members have been cloned that have only one Ig-variable domain (Shujian et al (1999); EP 0897981A1, incorporated herein by reference).

It is becoming apparent that inhibitory receptors are present on most of the hematopoietic cells, including dendritic cells, monocytes, CD19+ B cells; and CD3+ T cells (Borges and Cosman (2000) supra; De Maria et al., Proc. Natl. Acad. Sci. USA 94:10285-88 (1997), herein incorporated by reference in its entirety). An immunoreceptor expressed by mast cells is also known to downregulate cell activation signals (International Patent Application No. WO98/48017).

The receptors on NK and T cells have been shown to mediate innate immunity and play a major role in bone marrow graft rejection as well as in killing certain virus-infected and melanoma cells. Immunoglobulin receptors have also been implicated in mediating autoimmune reactions. More recently, they have also been shown to be required for development and maturation of dendritic cells (Fournier et al., J. Immunol. 165:1197-1209 (2000), herein incorporated by reference in its entirety). It has been shown that addition of an anti-Ig receptor monoclonal antibody to T cells induced their cytolytic activity for HIV infected target cells. It is apparent that the down regulation of an inhibitory receptor could lead to generalized activation of NK/T cells, which may cause autoimmune disorders like rheumatoid arthritis, multiple sclerosis (MS), systemic lupus erythematosus (SLE), psoriasis, and inflammatory bowel disease (IBD) among others.

Clearly, the activating and inhibitory signals mediated by opposing kinases and phosphatases are very important for maintaining balance in the immune systems. Systems with a predominance of activatory signals will lead to autoimmunity and inflammation. Immune systems with a predominance of inhibitory signals are less able to challenge infected cells or cancer cells. Isolating new activatory or inhibitory receptors is highly desirable for studying the biological signal(s) transduced via the receptor. Additionally, identifying such molecules provides a means of regulating and treating diseased states associated with autoimmunity, inflammation and infection.

For example, engaging a cell surface receptor having ITIM motifs, such as a KIRHy polypeptide, with an agonistic antibody or ligand can be used to downregulate a cell function in disease states in which the immune system is overactive and excessive inflammation or immunopathology is present. On the other hand, using an antagonistic antibody specific to KIRHy or a soluble form of KIRHy can be used to block the interaction of the cell surface receptor with the receptor's ligand to activate the specific immune function in disease states associated with suppressed immune function.

The KIRHy1 protein of the invention, a homolog of the human NK inhibitory receptor precursor (gi 20502982), is highly expressed in certain hematopoietic-based cancers, but not by most non-hematopoietic, healthy cells. Thus, targeting of cells that express KIRHy will have a minimal effect on healthy tissues while destroying or inhibiting the growth of cancer cells. Similarly, non-hematopoietic type tumors (i.e. solid tumors) can be targeted if they bear the KIRHy antigen. Targeting of KIRHy can also be used to treat disorders associated with the proliferation of KIRHy-expressing cells. Examples of disorders associated with the proliferation of KIRHy-expressing cells include cancers such as AML (also known as acute myelogenous leukemia, acute myelocytic leukemia, acute myeloblastic leukemia, acute granulocytic leukemia, and acute non-lymphocytic leukemia) and histiocytic lymphoma, In addition, non-Hodgkin's B cell lymphomas, B cell leukemias, T cell lymphomas, acute lymphoblastic leukemia (ALL), chronic myelogenous leukemia (CML), chronic myelomonocytic leukemia, B cell large cell lymphoma, multiple myeloma, myelodysplastic syndromes, hypereosinophilic syndrome, eosinophilic leukemia, X-linked proliferative disorders and Epstein Barr Virus-related conditions, such as mononucleosis; autoimmune disorders such as systemic lupus erythematosus; hyperproliferative disorders; organ and tissue transplant rejection; and certain allergic reactions. Non-hematopoietic tumors, such as breast colon, prostate, squamous cell or epithelial cell carcinomas that bear the KIRHy antigen can also be targeted.

KIRHy1 polypeptides and polynucleotides encoding such polypeptides are disclosed in co-owned U.S. patent application Ser. Nos. 09/631,451 and 09/491,404 which correspond to International Publication Nos. WO 01/55437 and WO 01/55437, respectively. These and all other U.S. patents and patent applications, foreign patents and International publications cited herein are hereby incorporated by reference in their entirety. U.S. patent application Ser. No. 09/491,404 incorporated by reference herein in its entirety relates, in general to a collection or library of at least one novel nucleic acid sequences, specifically contigs, assembled from expressed sequence tags (ESTs). U.S. patent application Ser. No. 09/631,451, incorporated by reference herein in its entirety, (specifically including all sequences in the sequence listing) discloses KIRHy polypeptides, isolated polynucleotides encoding such polypeptides, including recombinant molecules, cloned genes or degenerate variants thereof, especially naturally occurring variants such as allelic variants, fragments or analogs or variants of such polynucleotides or polypeptides, antisense polynucleotide molecules, and antibodies that specifically recognize one or more epitopes present on such polypeptides, including polyclonal, monoclonal, single chain, bispecific, fragment, human and humanized antibodies, as well as hybridomas producing monoclonal antibodies, and diagnostic and therapeutic uses and screening assays associated with such polynucleotides, polypeptides and antibodies.

The KIRHy1 polypeptide of SEQ ID NO: 3 is an approximately 305 amino acid protein with a predicted molecular weight of 33 kD unglycosylated. The initial methionine starts at position 114 of SEQ ID NO: 2 and the putative stop codon begins at position 1028 of SEQ ID NO: 2. A predicted 17 residue signal peptide is encoded from residue 1 to residue 17 of SEQ ID NO: 3 (i.e. SEQ ID NO: 4). The signal peptide region was predicted using the Neural Network SignalP V1.1 program (Nielsen et al., Int. J. Neural Syst. 8:581-599 (1997), herein incorporated by reference in its entirety). One of skill in the art will recognize that the actual cleavage site may be different than that predicted by the computer program. A predicted transmembrane domain is encoded from residue 171 to residue 193 of SEQ ID NO: 3 (i.e. SEQ ID NO: 6). The transmembrane domain was predicted using the Kyte-Doolittle hydrophobicity prediction algorithm (J. Mol. Biol. 157:105-131 (1982), herein incorporated by reference in their entirety). One of skill in the art will recognize that the actual domain may be different than that predicted by the computer program. Using the Pfam software program (Sonnhammer et al., Nucl. Acids Res. 26:320-322 (1998), herein incorporated by reference in its entirety), KIRHy1 is predicted to contain one immunoglobulin (Ig) domain spanning amino acids 33 to 110 (SEQ ID NO: 5). The soluble portion of KIRHy1 is represented by SEQ ID NO: 7.

The extracellular domain of KIRHy1 contains a single Ig-v domain (SEQ ID NO: 5) and numerous serines and threonines indicating it may be heavily O-glycosylated. Amino acids 188-191 contain a potential N-linked glycosylation site. The potential threonine O-linked glycosylation sites are at Thr²⁰, Thr²⁶, Thr¹²⁴, Thr¹³⁰, Thr¹³⁴, Thr¹³⁵, Thr¹³⁷, Thr¹³⁹, Thr¹⁴⁰, Thr¹⁴², Thr¹⁴⁶, and Thr¹⁵⁰. The potential serine O-lined glycosylation sites are Ser¹²⁹, Ser¹³⁸, Ser¹⁵¹, Ser¹⁵², and Ser¹⁵³. The cytoplasmic domain includes two ITIM motifs and one ITSM motif (immunoreceptor tyrosine-based switch motif) likely endowing it with inhibitory capability. Its putative transmembrane domain includes positively charged residues which may allow association with the activating receptor DAP12.

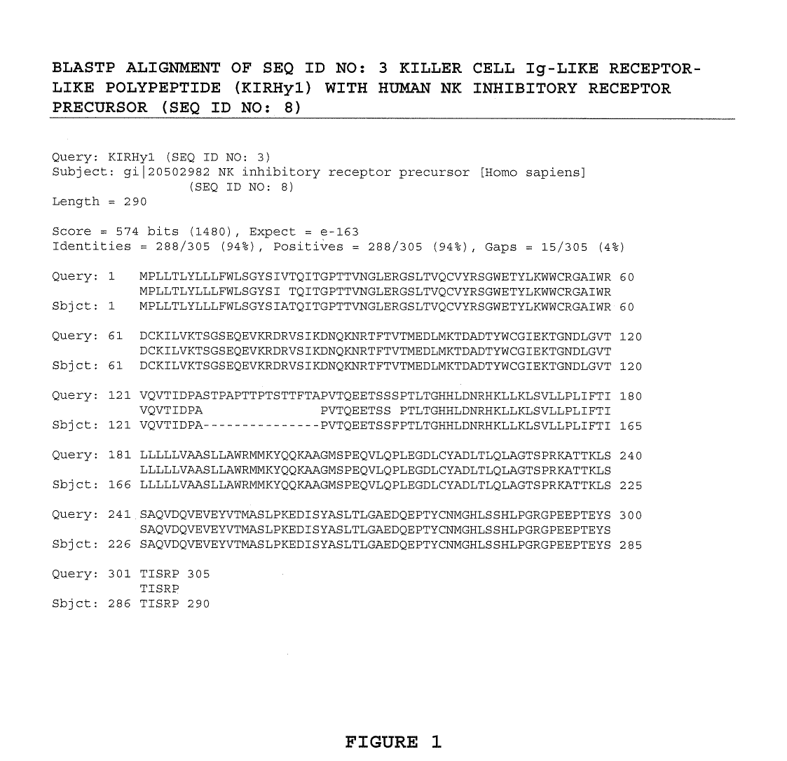

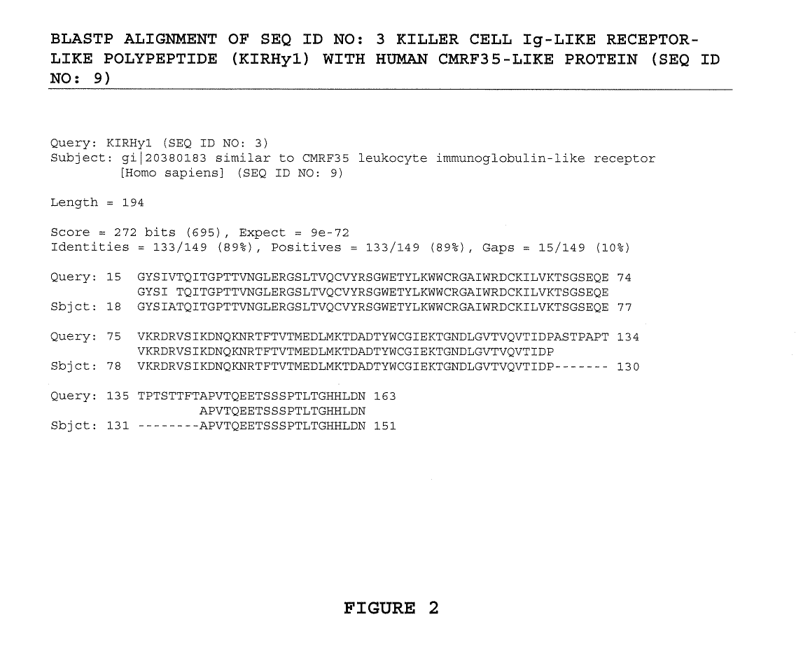

Protein database searches with the BLASTP algorithm (Altschul et al., J. Mol. Evol. 36:290-300 (1993); Altschul et al., J. Mol. Biol. 21:403-410 (1990), both of which are herein incorporated by reference in their entirety) indicate that SEQ ID NO: 3 is homologous to human NK inhibitory receptor precursor (gi 20502982) and a human CMRF35 homolog (“similar to CMRF35 leukocyte Ig-like receptor,” gi 20380183). An alignment of SEQ ID NO: 3 with human NK inhibitory receptor precursor (SEQ ID NO: 8) is shown in FIG. 1 indicating that the two sequences share 94% similarity and 94% identity over the entire amino acid sequence of SEQ ID NO: 3, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes. An alignment of SEQ ID NO: 3 with the CMRF35 homolog (SEQ ID NO: 9) is shown in FIG. 2, indicating that the two sequences share 89% similarity and 89% identity over 149 amino acids of SEQ ID NO: 3, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes. The gene corresponding to SEQ ID NO: 3 was localized to chromosome 17 (see Example 3). The CMRF35 family has also been localized to chromosome 17 near the psoriasis susceptibility locus (PSORS2), which may overlap with loci for atopic dermatitis and rheumatoid arthritis (Clark et al., Tissue Antigens 57:415-423 (2001); Speckman et al., Hum. Genet. 112:34-41 (2003), both of which are herein incorporated by reference in their entirety). Thus, KIRHy1 and the CMRF35 family members may play a role in psoriasis, atopic dermatitis and rheumatoid arthritis as well as in autoimmune diseases in general.

Due to alternative splicing, the KIRHy1 gene has several variants: KIRHy2 through KIRHy8 with four distinct C-terminal segments. The four possible C-terminal segments are: 1) PTEYSTISRP (SEQ ID NO: 26 within KIRHy1, KIRHy3, KIRHy6 and KIRHy7), 2) GYHEAFLCPG (SEQ ID NO: 27 within KIRHy5 and KIRHy8), 3) PWRATSAMQT (SEQ ID NO: 28 within KIRHy2) and 4) PTLTGHHLDN (SEQ ID NO: 29 within KIRHy4).

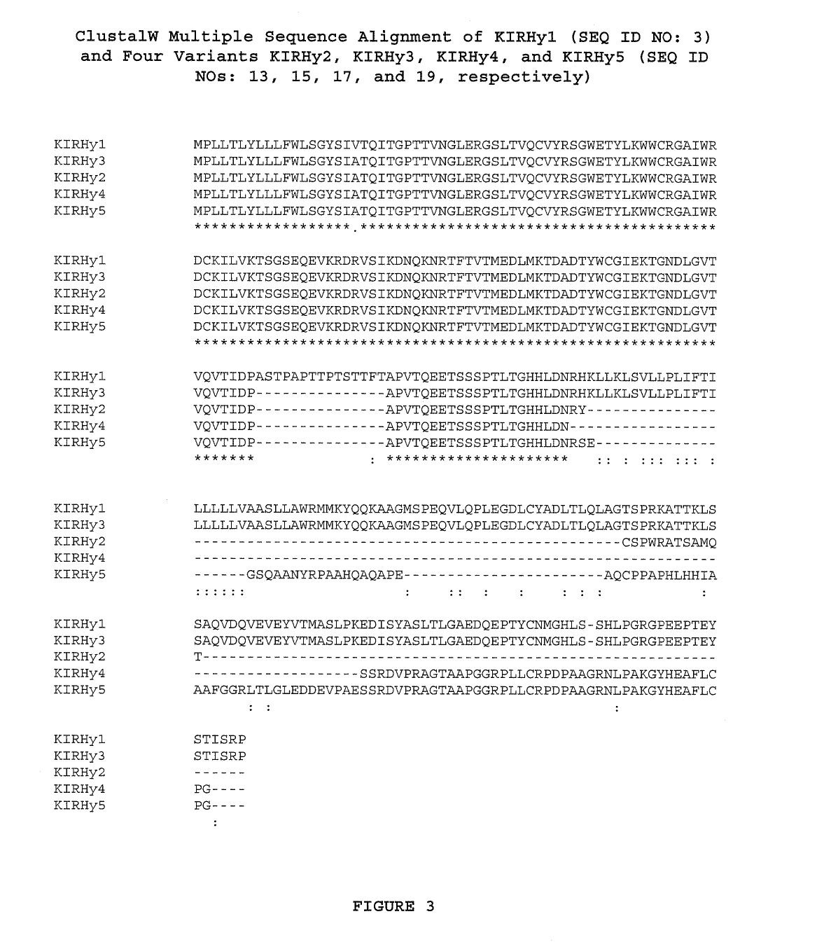

KIRHy2 (SEQ ID NO: 13) is an approximately 162 amino acid protein with a predicted molecular weight of 18 kD unglycosylated. The initial methionine starts at position 280 of SEQ ID NO: 12 and the putative stop codon begins at position 766 of SEQ ID NO: 12. FIG. 3 shows a multiple sequence alignment of KIRHy2 and KIRHy1 indicating that KIRHy2 lacks a portion of the KIRHy1 extracellular domain, namely residues 128-142 of SEQ ID NO: 3 and has a different transmembrane domain and cytoplasmic tail starting at residue 165 of SEQ ID NO: 3.

KIRHy3 (SEQ ID NO: 15) is an approximately 291 amino acid protein with a predicted molecular weight of 32 kD unglycosylated. The initial methionine starts at position 108 of SEQ ID NO: 14 and the putative stop codon begins at position 988 of SEQ ID NO: 14. FIG. 3 shows a multiple sequence alignment of KIRHy3 and KIRHy1 indicating that KIRHy3 lacks residues 128-142 of SEQ ID NO: 3.

KIRHy4 (SEQ ID NO: 17) is an approximately 192 amino acid protein with a predicted molecular weight of 21 kD unglycosylated. The initial methionine starts at position 108 of SEQ ID NO: 16 and the putative stop codon begins at position 681 of SEQ ID NO: 16. FIG. 3 shows a multiple sequence alignment of KIRHy4 and KIRHy1 indicating that KIRHy4 lacks residues 128-142 of SEQ ID NO: 3 and has a different transmembrane domain and cytoplasmic tail starting at residue 165 of SEQ ID NO: 3.

KIRHy5 (SEQ ID NO: 19) is an approximately 245 amino acid protein with a predicted molecular weight of 27 kD unglycosylated. The initial methionine starts at position 108 of SEQ ID NO: 18 and the putative stop codon begins at position 840 of SEQ ID NO: 18. FIG. 3 shows a multiple sequence alignment of KIRHy5 and KIRHy1 indicating that KIRHy5 lacks residues 128-142 of SEQ ID NO: 3 and has a similar transmembrane domain and cytoplasmic tail as that of SEQ ID NO: 17.

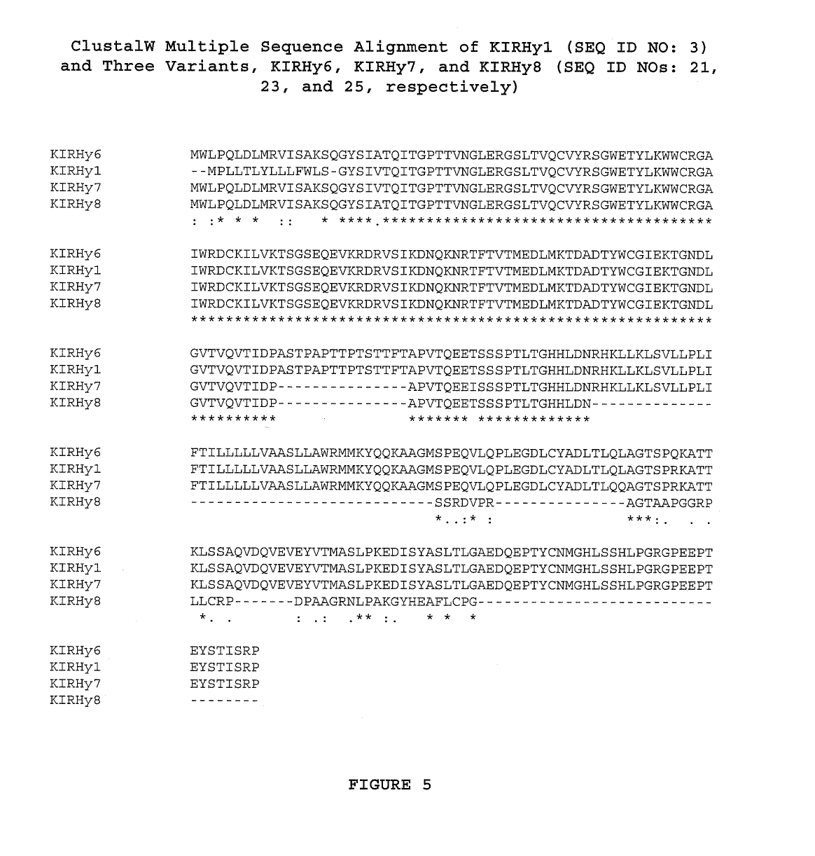

KIRHy6 (SEQ ID NO: 21) is an approximately 309 amino acid protein with a predicted molecular weight of 34 kD unglycosylated. The initial methionine starts at position 374 of SEQ ID NO: 20 and the putative stop codon begins at position 1298 of SEQ ID NO: 20. FIG. 5 shows a multiple sequence alignment of KIRHy6 and KIRHy1 indicating that KIRHy6 has a different start site than SEQ ID NO: 3.

KIRHy7 (SEQ ID NO: 23) is an approximately 294 amino acid protein with a predicted molecular weight of 32 kD unglycosylated. The initial methionine starts at position 264 of SEQ ID NO: 22 and the putative stop codon begins at position 1143 of SEQ ID NO: 22. FIG. 5 shows a multiple sequence alignment of KIRHy7 and KIRHy1 indicating that KIRHy7 lacks residues 127-142 of SEQ ID NO: 3.

KIRHy8 (SEQ ID NO: 25) is an approximately 195 amino acid protein with a predicted molecular weight of 21 kD unglycosylated. The initial methionine starts at position 278 of SEQ ID NO: 24 and the putative stop codon begins at position 860 of SEQ ID NO: 24. FIG. 5 shows a multiple sequence alignment of KIRHy8 and KIRHy1 indicating that KIRHy8 lacks residues 127-142 of SEQ ID NO: 3 and has a different transmembrane domain and cytoplasmic tail than that of SEQ ID NO: 3.

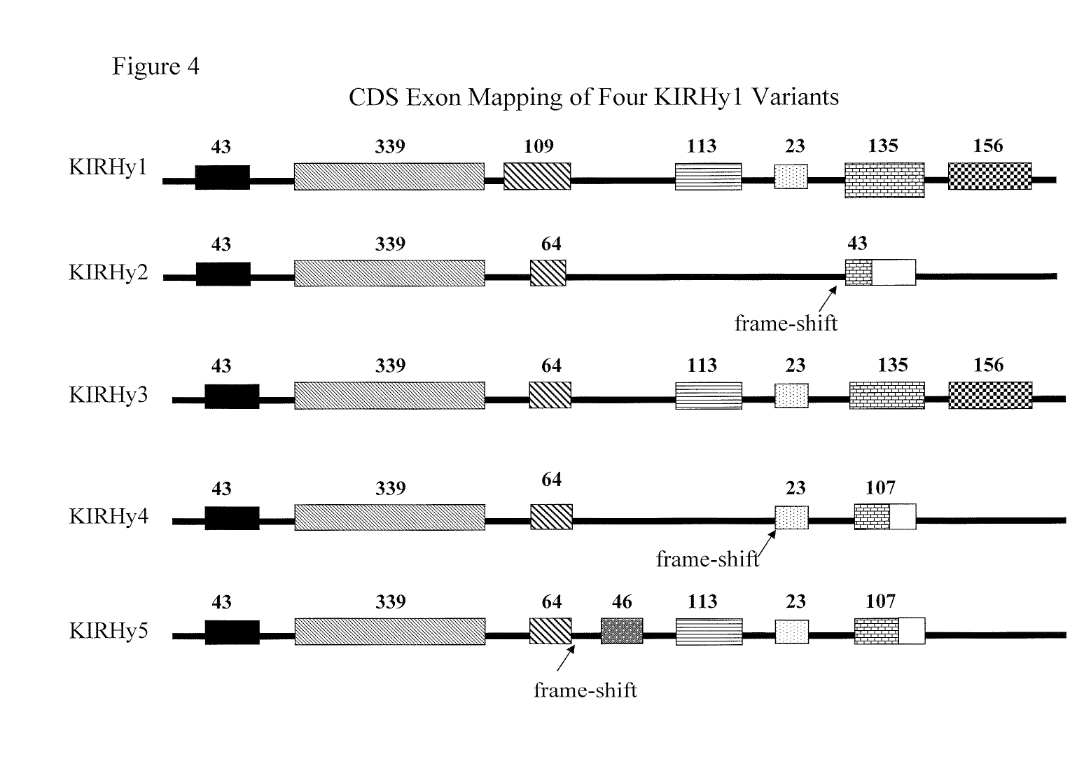

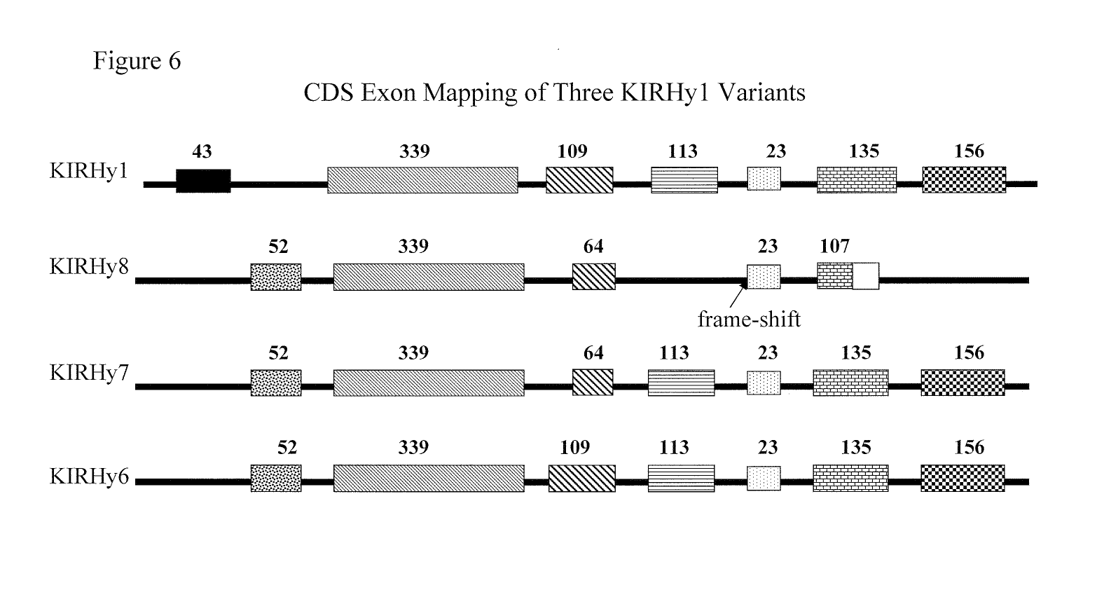

FIGS. 4 and 6 show schematics of the exon mapping for KIRHy1 and its variants including the sites of alternate splicing.

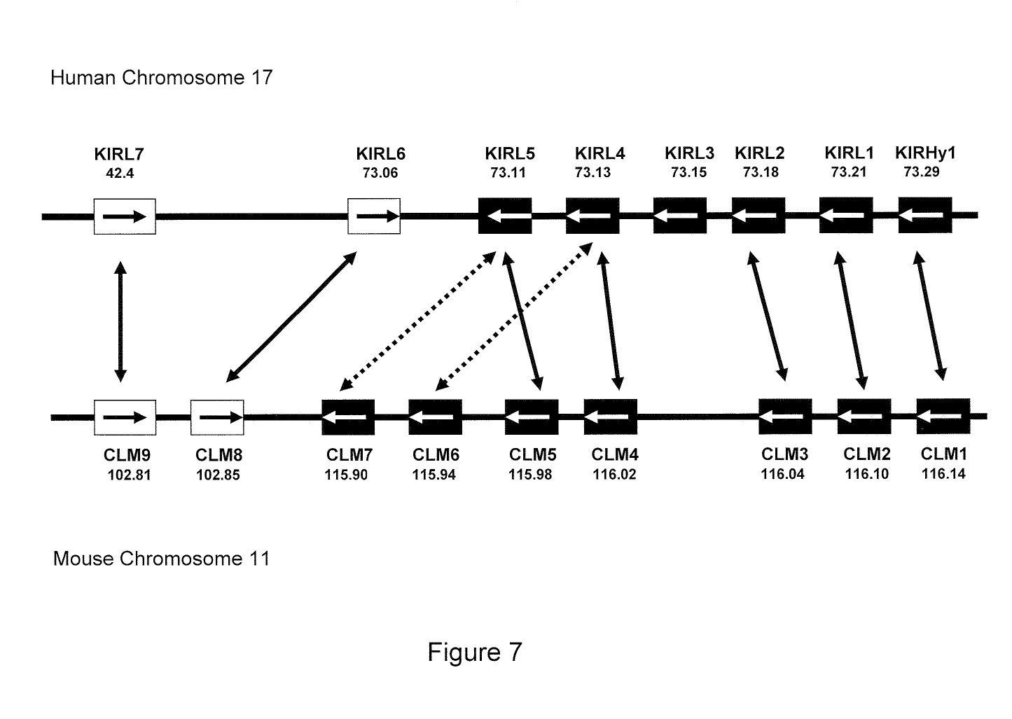

The mouse genome contains several CMRF-35-like genes (herein denoted CLM genes) that are clustered along murine chromosome 11. They are all single Ig domain containing proteins with a single transmembrane domain and a cytoplasmic domain with multiple tyrosine residues within the ITIM motifs. They are exclusively expressed in the myeloid lineage and CLM1 has been shown to inhibit osteoclast formation (Chung et al., J. Immunol. 171:6541-6548 (2003) herein incorporated by reference in its entirety). KIRHy1, the human ortholog of murine CMRF-35, has seven family members as well, all of which are clustered along human chromosome 17 and are herein denoted as KIRL1-7 for KIRHy1-Like proteins. The human homologs were identified by searching human gene databases (including the genome, dbEST, and Geneseq) based on their homology to the murine CLM genes. FIG. 7 shows the genomic organization of the CLM and KIRL gene clusters. In humans, it is localized on chromosome 17 within the [42.4-73.29] Mb range. In mice, it is localized on chromosome 11 in the [102.81-116.14] Mb range. There is no orthologous mapping for KIRL3, whereas KIRL6 and KIRL7 map to two genes each. Furthermore, the distance between 19457 and 19465 is significantly different than that between CLM9 and CLM8.

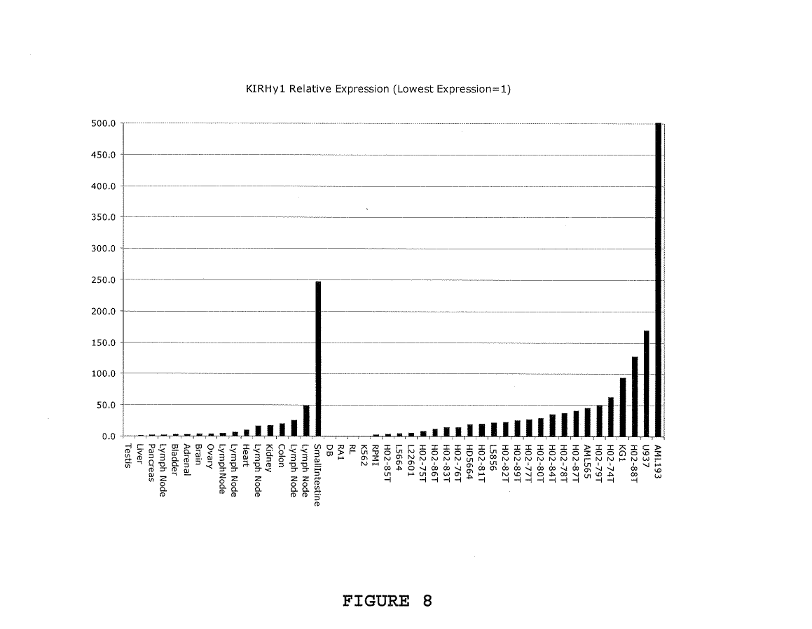

KIRHy1 is expressed in certain hematopoetic-based cancers, including AML and histiocytic lymphoma as well as B cell lymphoma, follicular lymphoma, diffuse large B cell lymphoma, anaplastic large T cell lymphoma, multiple myeloma, T cell leukemia, chronic myelogenous leukemia (CML), histiocytic lymphoma, plasmacytoma, non-Hodgkin's lymphoma, and Hodgkin's lymphoma, while most non-hematopoetic, healthy cells fail to express KIRHy or express it at low levels (see FIG. 8 and Table 3). Thus, targeting KIRHy will be useful in treating hematopoietic cancers such as AML and other hyperproliferative disorders.

KIRHy peptides can be used to target toxins or radioisotopes to tumor cells in vivo. KIRHy may be homophilic adhesion proteins which bind to itself. In this case the extracellular domain of a KIRHy polypeptide, or a fragment thereof, binds to cell surface KIRHy on tumor cells. This peptide fragment can be used as a means to deliver cytotoxic agents to KIRHy bearing tumor cells by specifically targeting cells expressing this antigen. Targeted delivery of these cytotoxic agents to the tumor cells would result in cell death and suppression of tumor growth. An example of the ability of an extracellular fragment binding to and activating its intact receptor by homophilic binding has been demonstrated with the CD84 receptor (Martin, et al., J. Immunol, 167:3668-3676 (2001), incorporated herein by reference in its entirety).

Extracellular fragments of the KIRHy receptor can be used to modulate immune cells expressing the protein by binding to and activating its own receptor expressed on the cell surface. On cells bearing a KIRHy receptor (such as NK cells, T cells, B cells and myeloid cells) this interaction results in stimulating the release of cytokines (such as interferon gamma for example) that, depending on the cytokine released, will enhance or suppress the immune system. Additionally, binding of these fragments to cells bearing the KIRHy receptor can activate these cells and thereby stimulate proliferation. Alternatively, some fragments upon binding to the intact KIRHy receptor will block activation signals and cytokine release by immune cells, thereby having an immune suppressive effect. Fragments that activate and stimulate the immune system may have anti-tumor properties by stimulating an immunological response resulting in immune mediated tumor cell killing. The same fragments may stimulate the immune system to mount an enhance response to foreign invaders such as virus and bacteria. Fragments that suppress the immune response can be useful in treating lymphoproliferative disorders, autoimmune disease, graft-vs-host disease, and inflammatory disorders such as emphysema.

5.2 Definitions

The term “fragment” of a nucleic acid refers to a sequence of nucleotide residues which are at least 5 nucleotides, more preferably at least 7 nucleotides, more preferably at least 9 nucleotides, more preferably at least 11 nucleotides and most preferably at least 17 nucleotides. The fragment is preferably less than 500 nucleotides, preferably less than 200 nucleotides, more preferably less than 100 nucleotides, more preferably less than 50 nucleotides and most preferably less than 30 nucleotides. Preferably the fragments can be used in polymerase chain reaction (PCR), various hybridization procedures or microarray procedures to identify or amplify identical or related parts of mRNA or DNA molecules. A fragment or segment may uniquely identify each polynucleotide sequence of the present invention. Preferably the fragment comprises a sequence substantially similar to a portion of SEQ ID NO: 1-2, 12, 14, 16, 18, 20, 22, 24, 30, 38, 40, 42, 46, 48, 50. A polypeptide “fragment” is a stretch of amino acid residues of at least 5 amino acids, preferably at least 7 amino acids, more preferably at least 9 amino acids and most preferably at least 17 or more amino acids. The peptide preferably is not greater than 200 amino acids, more preferably less than 150 amino acids and most preferably less than 100 amino acids. Preferably the peptide is from 5 to 200 amino acids. To be active, any polypeptide must have sufficient length to display biological and/or immunological activity. The term “immunogenic” refers to the capability of the natural, recombinant or synthetic KIRHy peptide, or any peptide thereof, to induce a specific immune response in appropriate animals or cells and to bind with specific antibodies.

The term “KIRHy antigen” refers to a molecule that when introduced into an animal is capable of stimulating an immune response in said animal specific to the KIRHy polypeptide or fragment thereof, of the present invention.

The term “variant” (or “analog”) refers to any polypeptide differing from naturally occurring polypeptides by amino acid insertions, deletions, and substitutions, created using, e.g., recombinant DNA techniques. Guidance in determining which amino acid residues may be replaced, added or deleted without abolishing activities of interest, may be found by comparing the sequence of the particular polypeptide with that of homologous peptides and minimizing the number of amino acid sequence changes made in regions of high homology (conserved regions) or by replacing amino acids with consensus sequence.

Alternatively, recombinant variants encoding these same or similar polypeptides may be synthesized or selected by making use of the “redundancy” in the genetic code. Various codon substitutions, such as the silent changes which produce various restriction sites, may be introduced to optimize cloning into a plasmid or viral vector or expression in a particular prokaryotic or eukaryotic system. Mutations in the polynucleotide sequence may be reflected in the polypeptide or domains of other peptides added to the polypeptide to modify the properties of any part of the polypeptide, to change characteristics such as ligand-binding affinities, interchain affinities, or degradation/turnover rate.

The term “stringent” is used to refer to conditions that are commonly understood in the art as stringent. Stringent conditions can include highly stringent conditions (i.e., hybridization to filter-bound DNA in 0.5 M NaHPO₄, 7% sodium dodecyl sulfate (SDS), 1 mM EDTA at 65° C., and washing in 0.1×SSC/0.1% SDS at 68° C.), and moderately stringent conditions (i.e., washing in 0.2×SSC/0.1% SDS at 42° C.). Other exemplary hybridization conditions are described herein in the examples.

In instances of hybridization of deoxyoligonucleotides, additional exemplary stringent hybridization conditions include washing in 6×SSC/0.05% sodium pyrophosphate at 37° C. (for 14-base oligonucleotides), 48° C. (for 17-base oligonucleotides), 55° C. (for 20-base oligonucleotides), and 60° C. (for 23-base oligonucleotides).

5.3 Targeting Using KIRHy Antigens

One embodiment of the present invention provides a composition comprising a KIRHy polypeptide to stimulate the immune system against KIRHy, thus targeting KIRHy-expressing cells. Use of a tumor antigen in a composition for generating cellular and humoral immunity for the purpose of anti-cancer therapy is well known in the art. For example, one type of tumor-specific antigen composition uses purified idiotype protein isolated from tumor cells, coupled to keyhole limpet hemocyanin (KLH) and mixed with adjuvant for injection into patients with low-grade follicular lymphoma (Hsu, et al., Blood 89: 3129-3135 (1997), herein incorporated by reference in its entirety). U.S. Pat. No. 6,312,718, herein incorporated by reference in its entirety, describes methods for inducing immune responses against malignant B cells, in particular lymphoma, chronic lymphocytic leukemia, and multiple myeloma. The methods described therein utilize vaccines that include liposomes having (1) at least one B-cell malignancy-associated antigen, (2) IL-2 alone, or in combination with at least one other cytokine or chemokine, and (3) at least one lipid molecule. Methods of targeting KIRHy using a KIRHy antigen typically employ a KIRHy polypeptide, including fragments, analogs and variants.

As another example, dendritic cells, one type of antigen-presenting cell, can be used in a cellular vaccine in which the dendritic cells are isolated from the patient, co-cultured with tumor antigen and then reinfused as a cellular vaccine (Hsu, et al., Nat. Med. 2:52-58 (1996), herein incorporated by reference in its entirety).

Combining this antigen therapy with other types of therapeutic agents in treatments such as chemotherapy or radiotherapy is also contemplated.

5.4 Targeting Using Nucleic Acids

5.4.1 Direct Delivery of Nucleic Acids

In some embodiments, a nucleic acid encoding KIRHy, or encoding a fragment, analog or variant thereof, within a recombinant vector is utilized. Such methods are known in the art. For example, immune responses can be induced by injection of naked DNA. Plasmid DNA that expresses bicistronic mRNA encoding both the light and heavy chains of tumor idiotype proteins, such as those from B cell lymphoma, when injected into mice, are able to generate a protective, anti-tumor response (Singh, et al., Vaccine 20:1400-1411 (2002), herein incorporated by reference in its entirety). KIRHy viral vectors are particularly useful for delivering nucleic acids encoding KIRHy of the invention to cells. Examples of vectors include those derived from influenza, adenovirus, vaccinia, herpes symplex virus, fowlpox, vesicular stomatitis virus, canarypox, poliovirus, adeno-associated virus, and lentivirus and sindbus virus. Of course, non-viral vectors, such as liposomes or even naked DNA, are also useful for delivering nucleic acids encoding KIRHy of the invention to cells.

Combining this type of therapy with other types of therapeutic agents or treatments such as chemotherapy or radiation is also contemplated.

5.4.2 Nucleic Acids Expressed in Cells

In some embodiments, a vector comprising a nucleic acid encoding the KIRHy polypeptide (including a fragment, analog or variant) is introduced into a cell, such as a dendritic cell or a macrophage. When expressed in an antigen-presenting cell (APC), the KIRHy cell surface antigens are presented to T cells eliciting an immune response against KIRHy. Such methods are also known in the art. Methods of introducing tumor antigens into APCs and vectors useful therefor are described in U.S. Pat. No. 6,300,090, herein incorporated by reference in its entirety. The vector encoding KIRHy may be introduced into the APCs in vivo. Alternatively, APCs are loaded with KIRHy or a nucleic acid encoding KIRHy ex vivo and then introduced into a patient to elicit an immune response against KIRHy. In another alternative, the cells presenting KIRHy antigen are used to stimulate the expansion of anti-KIRHy cytotoxic T lymphocytes (CTL) ex vivo followed by introduction of the stimulated CTL into a patient. (U.S. Pat. No. 6,306,388, herein incorporated by reference in its entirety).

Combining this type of therapy with other types of therapeutic agents or treatments such as chemotherapy or radiation is also contemplated.

5.4.3 Antisense Nucleic Acids

Another aspect of the invention pertains to isolated antisense nucleic acid molecules that can hybridize to, or are complementary to, the nucleic acid molecule comprising the KIRHy nucleotide sequence, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least 10, 25, 50, 100, 250 or 500 nucleotides or an entire KIRHy coding strand, or to only a portion thereof. Nucleic acid molecules encoding fragments, homologs, derivatives and analogs of a KIRHy or antisense nucleic acids complementary to a KIRHy nucleic acid sequence of are additionally provided.

In one embodiment, an antisense nucleic acid molecule is antisense to a “coding region” of the coding strand of a nucleotide sequence encoding a KIRHy protein. The term “coding region” refers to the region of the nucleotide sequence comprising codons which are translated into amino acid residues. In another embodiment, the antisense nucleic acid molecule is antisense to a “conceding region” of the coding strand of a nucleotide sequence encoding the KIRHy protein. The term “conceding region” refers to 5′ and 3′ sequences which flank the coding region that are not translated into amino acids (i.e., also referred to as 5′ and 3′ untranslated regions).

Given the coding strand sequences encoding the KIRHy protein disclosed herein, antisense nucleic acids of the invention can be designed according to the rules of Watson and Crick or Hoogsteen base pairing. The antisense nucleic acid molecule can be complementary to the entire coding region of KIRHy mRNA, but more preferably is an oligonucleotide that is antisense to only a portion of the coding or noncoding region of KIRHy mRNA. For example, the antisense oligonucleotide can be complementary to the region surrounding the translation start site of KIRHy mRNA. An antisense oligonucleotide can be, for example, 5, 10, 15, 20, 25, 30, 35, 40, 45, or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following section).

The antisense nucleic acid molecules of the invention are typically administered to a subject or generated in situ such that they hybridize with or bind to cellular mRNA and/or genomic DNA encoding a KIRHy protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an alpha-anomeric nucleic acid molecule. An alpha-anomeric nucleic acid molecule forms specific double-stranded hybrids with complementary RNA in which, contrary to the usual alpha-units, the strands run parallel to each other. See, e.g., Gaultier, et al., Nucl. Acids Res. 15: 6625-6641 (1987). The antisense nucleic acid molecule can also comprise a 2′-o-methylribonucleotide (see, e.g., Inoue, et al., Nucl. Acids Res. 15: 6131-6148 (1987)) or a chimeric RNA-DNA analogue (see, e.g., Inoue, et al., FEBS Lett. 215: 327-330 (1987), all of which are herein incorporated by reference in their entirety.

5.4.4 Ribozymes and PNA Moieties

In still another embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave mRNA transcripts to thereby inhibit translation of an mRNA. A ribozyme having specificity for a nucleic acid of the invention can be designed based upon the nucleotide sequence of a DNA disclosed herein (i.e., SEQ ID NO: 1-2, 12, 14, 16, 18, 20, 22, 24, 30, 38, 40, 42, 46, 48, 50). For example, a derivative of Tetrahymena L-19 IVS RNA can be constructed in which the nucleotide sequence of the active site is complementary to the nucleotide sequence to be cleaved in a mRNA. See, e.g., Cech et al. U.S. Pat. No. 4,987,071; and Cech et al. U.S. Pat. No. 5,116,742. Alternatively, mRNA of the invention can be used to select a catalytic RNA having a specific ribonuclease activity from a pool of RNA molecules. See, e.g., Bartel et al., (1993) Science 261:1411-1418.

Alternatively, gene expression can be inhibited by targeting nucleotide sequences complementary to the regulatory region (e.g., promoter and/or enhancers) to form triple helical structures that prevent transcription of the gene in target cells. See generally, Helene. (1991) Anticancer Drug Des. 6: 569-84; Helene. et al. (1992) Ann. N.Y. Acad. Sci. 660:27-36; and Maher (1992) Bioassays 14: 807-15.

In various embodiments, the nucleic acids of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorg Med Chem 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) PNAS 93: 14670-675.

PNAs of the invention can be used in therapeutic and diagnostic applications. For example, PNAs can be used as antisense or antigene agents for sequence-specific modulation of gene expression by, e.g., inducing transcription or translation arrest or inhibiting replication. PNAs of the invention can also be used, e.g., in the analysis of single base pair mutations in a gene by, e.g., PNA directed PCR clamping; as artificial restriction enzymes when used in combination with other enzymes, e.g., S1 nucleases (Hyrup B. (1996) above); or as probes or primers for DNA sequence and hybridization (Hyrup et al. (1996), above; Perry-O'Keefe (1996), above).

In another embodiment, PNAs of the invention can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) above). The synthesis of PNA-DNA chimeras can be performed as described in Hyrup (1996) above and Finn et al. (1996) Nucl Acids Res 24: 3357-63. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Mag et al. (1989) Nucl Acid Res 17: 5973-88). PNA monomers are then coupled in a stepwise manner to produce a chimeric molecule with a 5′ PNA segment and a 3′ DNA segment (Finn et al. (1996) above). Alternatively, chimeric molecules can be synthesized with a 5′ DNA segment and a 3′ PNA segment. See, Petersen et al. (1975) Bioorg Med Chem Lett 5: 1119-11124.

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810) or the blood-brain barrier (see, e.g., PCT Publication No. WO89/10134). In addition, oligonucleotides can be modified with hybridization triggered cleavage agents (See, e.g., Krol et al., 1988, BioTechniques 6:958-976) or intercalating agents. (See, e.g., Zon, 1988, Pharm. Res. 5: 539-549). To this end, the oligonucleotide may be conjugated to another molecule, e.g., a peptide, a hybridization triggered cross-linking agent, a transport agent, a hybridization-triggered cleavage agent, etc.

5.4.5 KIRHy Nucleic Acids

The isolated polynucleotides of the invention include, but are not limited to a polynucleotide comprising any of the nucleotide sequences of SEQ ID NO: 1-2, 12, 14, 16, 18, 20, 22, 24, 30, 38, 40, 42, 44, 46, 48, 50; a fragment of SEQ ID NO: 1-2, 12, 14, 16, 18, 20, 22, 24, 30, 38, 40, 42, 44, 46, 48, 50; a polynucleotide comprising the full length protein coding sequence of SEQ ID NO: 1-2, 12, 14, 16, 18, 20, 22, 24, 30, 38, 40, 42, 44, 46, 48, 50 (for example coding for SEQ ID NO: 3); and a polynucleotide comprising the nucleotide sequence encoding the mature protein sequence of the polypeptides of any one of SEQ ID NO: 1-2, 12, 14, 16, 18, 20, 22, 24, 30, 38, 40, 42, 44, 46, 48, 50. The polynucleotides of the present invention also include, but are not limited to, a polynucleotide that hybridizes under stringent conditions to (a) the complement of any of the nucleotides sequences of SEQ ID NO: 1-2, 12, 14, 16, 18, 20, 22, 24, 30, 38, 40, 42, 44, 46, 48, 50; (b) a polynucleotide encoding any one of the polypeptides of SEQ ID NO: 3-7, 11, 13, 15, 17, 19, 21, 23, 25-29, 31-37, 39, 41, 43, 45, 47, 49, 51; (c) a polynucleotide which is an allelic variant of any polynucleotides recited above; (d) a polynucleotide which encodes a species homolog of any of the proteins recited above; or (e) a polynucleotide that encodes a polypeptide comprising a specific domain or truncation of the polypeptides of SEQ ID NO: 3-7, 11, 13, 15, 17, 19, 21, 23, 25-29, 31-37, 39, 41, 43, 45, 47, 49, 51. Domains of interest may depend on the nature of the encoded polypeptide; e.g., domains in receptor-like polypeptides include ligand-binding, extracellular, transmembrane, or cytoplasmic domains, or combinations thereof; domains in immunoglobulin-like proteins include the variable immunoglobulin-like domains; domains in enzyme-like polypeptides include catalytic and substrate binding domains; and domains in ligand polypeptides include receptor-binding domains.

The polynucleotides of the invention include naturally occurring or wholly or partially synthetic DNA, e.g., cDNA and genomic DNA, and RNA, e.g., mRNA. The polynucleotides may include the entire coding region of the cDNA or may represent a portion of the coding region of the cDNA.

The present invention also provides genes corresponding to the cDNA sequences disclosed herein. The corresponding genes can be isolated in accordance with known methods using the sequence information disclosed herein. Such methods include the preparation of probes or primers from the disclosed sequence information for identification and/or amplification of genes in appropriate genomic libraries or other sources of genomic materials. Further 5′ and 3′ sequence can be obtained using methods known in the art. For example, full length cDNA or genomic DNA that corresponds to any of the polynucleotides of SEQ ID NO: 1-2, 12, 14, 16, 18, 20, 22, 24, 30, 38, 40, 42, 44, 46, 48, 50 can be obtained by screening appropriate cDNA or genomic DNA libraries under suitable hybridization conditions using any of the polynucleotides of SEQ ID NO: 1-2, 12, 14, 16, 18, 20, 22, 24, 30, 38, 40, 42, 44, 46, 48, 50 or a portion thereof as a probe. Alternatively, the polynucleotides of SEQ ID NO: 1-2, 12, 14, 16, 18, 20, 22, 24, 30, 38, 40, 42, 44, 46, 48, 50 may be used as the basis for suitable primer(s) that allow identification and/or amplification of genes in appropriate genomic DNA or cDNA libraries.

The nucleic acid sequences of the invention can be assembled from ESTs and sequences (including cDNA and genomic sequences) obtained from one or more public databases, such as dbEST, gbpri, and UniGene. The EST sequences can provide identifying sequence information, representative fragment or segment information, or novel segment information for the full-length gene.

The polynucleotides of the invention also provide polynucleotides including nucleotide sequences that are substantially equivalent to the polynucleotides recited above. Polynucleotides according to the invention can have, e.g., at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98% or 99% sequence identity to a polynucleotide recited above.

Included within the scope of the nucleic acid sequences of the invention are nucleic acid sequence fragments that hybridize under stringent conditions to any of the nucleotide sequences of SEQ ID NO: 1-2, 12, 14, 16, 18, 20, 22, 24, 30, 38, 40, 42, 44, 46, 48, 50, or complements thereof, which fragment is greater than 5 nucleotides, preferably 7 nucleotides, more preferably greater than 9 nucleotides and most preferably greater than 17 nucleotides. Fragments of, e.g. 15, 17, or 20 nucleotides or more that are selective for (i.e. specifically hybridize to any one of the polynucleotides of the invention) are contemplated. Probes capable of specifically hybridizing to a polynucleotide can differentiate polynucleotide sequences of the invention from other polynucleotide sequences in the same family of genes or can differentiate human genes from genes of other species, and are preferably based on unique nucleotide sequences.

The sequences falling within the scope of the present invention are not limited to these specific sequences, but also include allelic and species variations thereof. Allelic and species variations can be routinely determined by comparing the sequence provided in SEQ ID NO: 1-2, 12, 14, 16, 18, 20, 22, 24, 30, 38, 40, 42, 44, 46, 48, 50, a representative fragment thereof, or a nucleotide sequence at least 90% identical, preferably 95% identical, to SEQ ID NO: 1-2, 12, 14, 16, 18, 20, 22, 24, 30, 38, 40, 42, 44, 46, 48, 50 with a sequence from another isolate of the same species. Furthermore, to accommodate codon variability, the invention includes nucleic acid molecules coding for the same amino acid sequences as do the specific ORFs disclosed herein. In other words, in the coding region of an ORF, substitution of one codon for another codon that encodes the same amino acid is expressly contemplated.

The nearest neighbor result for the nucleic acids of the present invention, including SEQ ID NO: 1-2, 12, 14, 16, 18, 20, 22, 24, 30, 38, 40, 42, 44, 46, 48, 50, can be obtained by searching a database using an algorithm or a program. Preferably, a BLAST which stands for Basic Local Alignment Search Tool is used to search for local sequence alignments (Altshul, S. F., J. Mol. Evol. 36 290-300 (1993) and Altschul S. F., et al. J. Mol. Biol. 21:403-410 (1990)).

Species homologs (or orthologs) of the disclosed polynucleotides and proteins are also provided by the present invention. Species homologs may be isolated and identified by making suitable probes or primers from the sequences provided herein and screening a suitable nucleic acid source from the desired species.

The invention also encompasses allelic variants of the disclosed polynucleotides or proteins; that is, naturally-occurring alternative forms of the isolated polynucleotide which also encodes proteins which are identical, homologous or related to that encoded by the polynucleotides.

The nucleic acid sequences of the invention are further directed to sequences which encode variants of the described nucleic acids. These amino acid sequence variants may be prepared by methods known in the art by introducing appropriate nucleotide changes into a native or variant polynucleotide. There are two variables in the construction of amino acid sequence variants: the location of the mutation and the nature of the mutation. Nucleic acids encoding the amino acid sequence variants are preferably constructed by mutating the polynucleotide to encode an amino acid sequence that does not occur in nature. These nucleic acid alterations can be made at sites that differ in the nucleic acids from different species (variable positions) or in highly conserved regions (constant regions). Sites at such locations will typically be modified in series, e.g., by substituting first with conservative choices (e.g., hydrophobic amino acid to a different hydrophobic amino acid) and then with more distant choices (e.g., hydrophobic amino acid to a charged amino acid), and then deletions or insertions may be made at the target site. Amino acid sequence deletions generally range from 1 to 30 residues, preferably 1 to 10 residues, and are typically contiguous. Amino acid insertions include amino- and/or carboxyl-terminal fusions ranging in length from one to one hundred or more residues, as well as intrasequence insertions of single or multiple amino acid residues. Intrasequence insertions may range generally from 1 to 10 amino residues, preferably from 1 to 5 residues. Examples of terminal insertions include the heterologous signal sequences necessary for secretion or for intracellular targeting in different host cells and sequences such as FLAG® or poly-histidine sequences useful for purifying the expressed protein.

In a preferred method, polynucleotides encoding the novel amino acid sequences are changed via site-directed mutagenesis. This method uses oligonucleotide sequences to alter a polynucleotide to encode the desired amino acid variant, as well as sufficient adjacent nucleotides on both sides of the changed amino acid to form a stable duplex on either side of the site being changed. In general, the techniques of site-directed mutagenesis are well known to those of skill in the art and this technique is exemplified by publications such as, Edelman et al., DNA 2:183 (1983). A versatile and efficient method for producing site-specific changes in a polynucleotide sequence was published by Zoller and Smith, Nucleic Acids Res. 10:6487-6500 (1982). PCR may also be used to create amino acid sequence variants of the novel nucleic acids. When small amounts of template DNA are used as starting material, primer(s) that differs slightly in sequence from the corresponding region in the template DNA can generate the desired amino acid variant. PCR amplification results in a population of product DNA fragments that differ from the polynucleotide template encoding the polypeptide at the position specified by the primer. The product DNA fragments replace the corresponding region in the plasmid and this gives a polynucleotide encoding the desired amino acid variant.

A further technique for generating amino acid variants is the cassette mutagenesis technique described in Wells, et al., Gene 34:315 (1985); and other mutagenesis techniques well known in the art, such as, for example, the techniques in Sambrook, et al., supra, and Current Protocols in Molecular Biology, Ausubel, et al. Due to the inherent degeneracy of the genetic code, other DNA sequences which encode substantially the same or a functionally equivalent amino acid sequence may be used in the practice of the invention for the cloning and expression of these novel nucleic acids. Such DNA sequences include those which are capable of hybridizing to the appropriate novel nucleic acid sequence under stringent conditions.

Polynucleotides encoding preferred polypeptide truncations of the invention can be used to generate polynucleotides encoding chimeric or fusion proteins comprising one or more domains of the invention and heterologous protein sequences.

The polynucleotides of the invention additionally include the complement of any of the polynucleotides recited above. The polynucleotide can be DNA (genomic, cDNA, amplified, or synthetic) or RNA. Methods and algorithms for obtaining such polynucleotides are well known to those of skill in the art and can include, for example, methods for determining hybridization conditions that can routinely isolate polynucleotides of the desired sequence identities.

In accordance with the invention, polynucleotide sequences comprising the mature protein sequences, coding for any one of SEQ ID NO: 3, 11, 13, 15, 17, 19, 21, 23, 25, 31, 39, 41, 43, 45, 47, 49, 51, or functional equivalents thereof, may be used to generate recombinant DNA molecules that direct the expression of that nucleic acid, or a functional equivalent thereof, in appropriate host cells. Also included are the cDNA inserts of any of the clones identified herein.

A polynucleotide according to the invention can be joined to any of a variety of other nucleotide sequences by well-established recombinant DNA techniques (see Sambrook, J. et al. (1989) Molecular Cloning: A Laboratory Manual, Cold Spring Harbor Laboratory, NY). Useful nucleotide sequences for joining to polynucleotides include an assortment of vectors, e.g., plasmids, cosmids, lambda phage derivatives, phagemids, and the like, that are well known in the art. Accordingly, the invention also provides a vector including a polynucleotide of the invention and a host cell containing the polynucleotide. In general, the vector contains an origin of replication functional in at least one organism, convenient restriction endonuclease sites, and a selectable marker for the host cell. Vectors according to the invention include expression vectors, replication vectors, probe generation vectors, and sequencing vectors. A host cell according to the invention can be a prokaryotic or eukaryotic cell and can be a unicellular organism or part of a multicellular organism.

The present invention further provides recombinant constructs comprising a nucleic acid having any of the nucleotide sequences of SEQ ID NO: 1-2, 12, 14, 16, 18, 20, 22, 24, 30, 38, 40, 42, 44, 46, 48, 50 or a fragment thereof or any other polynucleotides of the invention. In one embodiment, the recombinant constructs of the present invention comprise a vector, such as a plasmid or viral vector, into which a nucleic acid having any of the nucleotide sequences of SEQ ID NO: 1-2, 12, 14, 16, 18, 20, 22, 24, 30, 38, 40, 42, 44, 46, 48, 50 or a fragment thereof is inserted, in a forward or reverse orientation. In the case of a vector comprising one of the ORFs of the present invention, the vector may further comprise regulatory sequences, including for example, a promoter, operably linked to the ORF. Large numbers of suitable vectors and promoters are known to those of skill in the art and are commercially available for generating the recombinant constructs of the present invention. The following vectors are provided by way of example. Bacterial: pBs, phagescript, PsiX174, pBluescript SK, pBs KS, pNH8a, pNH16a, pNH18a, pNH46a (Stratagene); pTrc99A, pKK223-3, pKK233-3, pDR540, pRIT5 (Pharmacia). Eukaryotic: pWLneo, pSV2cat, pOG44, PXTI, pSG (Stratagene) pSVK3, pBPV, pMSG, and pSVL (Pharmacia).

The isolated polynucleotide of the invention may be operably linked to an expression control sequence such as the pMT2 or pED expression vectors disclosed in Kaufman et al., Nucleic Acids Res. 19:4485-4490 (1991), in order to produce the protein recombinantly. Many suitable expression control sequences are known in the art. General methods of expressing recombinant proteins are also known and are exemplified in R. Kaufman, Methods in Enzymology 185:537-566 (1990). As defined herein “operably linked” means that the isolated polynucleotide of the invention and an expression control sequence are situated within a vector or cell in such a way that the protein is expressed by a host cell which has been transformed (transfected) with the ligated polynucleotide/expression control sequence.

Promoter regions can be selected from any desired gene using CAT (chloramphenicol transferase) vectors or other vectors with selectable markers. Two appropriate vectors are pKK232-8 and pCM7. Particular named bacterial promoters include lacI, lacZ, T3, T7, gpt, lambda PR, and trc. Eukaryotic promoters include CMV immediate early, HSV thymidine kinase, early and late SV40, LTRs from retrovirus, and mouse metallothionein-1. Selection of the appropriate vector and promoter is well within the level of ordinary skill in the art. Generally, recombinant expression vectors will include origins of replication and selectable markers permitting transformation of the host cell, e.g., the ampicillin resistance gene of E. coli and S. cerevisiae TRP1 gene, and a promoter derived from a highly expressed gene to direct transcription of a downstream structural sequence. Such promoters can be derived from operons encoding glycolytic enzymes such as 3-phosphoglycerate kinase (PGK), a-factor, acid phosphatase, or heat shock proteins, among others. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an amino terminal identification peptide imparting desired characteristics, e.g., stabilization or simplified purification of expressed recombinant product. Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and to, if desirable, provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice.

As a representative but non-limiting example, useful expression vectors for bacterial use can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and GEM 1 (Promega Biotech, Madison, Wis., USA). These pBR322 “backbone” sections are combined with an appropriate promoter and the structural sequence to be expressed. Following transformation of a suitable host strain and growth of the host strain to an appropriate cell density, the selected promoter is induced or derepressed by appropriate means (e.g., temperature shift or chemical induction) and cells are cultured for an additional period. Cells are typically harvested by centrifugation, disrupted by physical or chemical means, and the resulting crude extract retained for further purification.

Polynucleotides of the invention can also be used to induce immune responses. For example, as described in Fan, et al., Nat. Biotech. 17:870-872 (1999), incorporated herein by reference, nucleic acid sequences encoding a polypeptide may be used to generate antibodies against the encoded polypeptide following topical administration of naked plasmid DNA or following injection, and preferably intramuscular injection of the DNA. The nucleic acid sequences are preferably inserted in a recombinant expression vector and may be in the form of naked DNA.

5.4.6 Gene Therapy

Mutations in the polynucleotides of the invention gene may result in loss of normal function of the encoded protein. The invention thus provides gene therapy to restore normal activity of the polypeptides of the invention; or to treat disease states involving polypeptides of the invention. Delivery of a functional gene encoding polypeptides of the invention to appropriate cells is effected ex vivo, in situ, or in vivo by use of vectors, and more particularly viral vectors (e.g., adenovirus, adeno-associated virus, or a retrovirus), or ex vivo by use of physical DNA transfer methods (e.g., liposomes or chemical treatments). See, for example, Anderson, Nature, 392(Suppl):25-20 (1998). For additional reviews of gene therapy technology see Friedmann, Science, 244: 1275-1281 (1989); Verma, Scientific American: 68-84 (1990); and Miller, Nature, 357: 455-460 (1992), all of which are herein incorporated by reference in their entirety. Introduction of any one of the nucleotides of the present invention or a gene encoding the polypeptides of the present invention can also be accomplished with extrachromosomal substrates (transient expression) or artificial chromosomes (stable expression). Cells may also be cultured ex vivo in the presence of proteins of the present invention in order to proliferate or to produce a desired effect on or activity in such cells. Treated cells can then be introduced in vivo for therapeutic purposes. Alternatively, it is contemplated that in other human disease states, preventing the expression of or inhibiting the activity of polypeptides of the invention will be useful in treating the disease states. It is contemplated that antisense therapy or gene therapy could be applied to negatively regulate the expression of polypeptides of the invention.

Other methods of inhibiting expression of a protein include the introduction of antisense molecules to the nucleic acids of the present invention, their complements, or their translated RNA sequences, by methods known in the art. Further, the polypeptides of the present invention can be inhibited by using targeted deletion methods, or the insertion of a negative regulatory element such as a silencer, which is tissue specific.

The present invention still further provides cells genetically engineered in vivo to express the polynucleotides of the invention, wherein such polynucleotides are in operative association with a regulatory sequence heterologous to the host cell which drives expression of the polynucleotides in the cell. These methods can be used to increase or decrease the expression of the polynucleotides of the present invention.

Knowledge of DNA sequences provided by the invention allows for modification of cells to permit, increase, or decrease, expression of endogenous polypeptide. Cells can be modified (e.g., by homologous recombination) to provide increased polypeptide expression by replacing, in whole or in part, the naturally occurring promoter with all or part of a heterologous promoter so that the cells express the protein at higher levels. The heterologous promoter is inserted in such a manner that it is operatively linked to the desired protein encoding sequences. See, for example, PCT International Publication No. WO 94/12650, PCT International Publication No. WO 92/20808, and PCT International Publication No. WO 91/09955, all of which are incorporated by reference in their entirety. It is also contemplated that, in addition to heterologous promoter DNA, amplifiable marker DNA (e.g., ada, dhfr, and the multifunctional CAD gene which encodes carbamyl phosphate synthase, aspartate transcarbamylase, and dihydroorotase) and/or intron DNA may be inserted along with the heterologous promoter DNA. If linked to the desired protein coding sequence, amplification of the marker DNA by standard selection methods results in co-amplification of the desired protein coding sequences in the cells.

In another embodiment of the present invention, cells and tissues may be engineered to express an endogenous gene comprising the polynucleotides of the invention under the control of inducible regulatory elements, in which case the regulatory sequences of the endogenous gene may be replaced by homologous recombination. As described herein, gene targeting can be used to replace a gene's existing regulatory region with a regulatory sequence isolated from a different gene or a novel regulatory sequence synthesized by genetic engineering methods. Such regulatory sequences may be comprised of promoters, enhancers, scaffold-attachment regions, negative regulatory elements, transcriptional initiation sites, regulatory protein binding sites or combinations of said sequences. Alternatively, sequences which affect the structure or stability of the RNA or protein produced may be replaced, removed, added, or otherwise modified by targeting. These sequences include polyadenylation signals, mRNA stability elements, splice sites, leader sequences for enhancing or modifying transport or secretion properties of the protein, or other sequences which alter or improve the function or stability of protein or RNA molecules.

The targeting event may be a simple insertion of the regulatory sequence, placing the gene under the control of the new regulatory sequence, e.g., inserting a new promoter or enhancer or both upstream of a gene. Alternatively, the targeting event may be a simple deletion of a regulatory element, such as the deletion of a tissue-specific negative regulatory element. Alternatively, the targeting event may replace an existing element; for example, a tissue-specific enhancer can be replaced by an enhancer that has broader or different cell-type specificity than the naturally occurring elements. Here, the naturally occurring sequences are deleted and new sequences are added. In all cases, the identification of the targeting event may be facilitated by the use of one or more selectable marker genes that are contiguous with the targeting DNA, allowing for the selection of cells in which the exogenous DNA has integrated into the cell genome. The identification of the targeting event may also be facilitated by the use of one or more marker genes exhibiting the property of negative selection, such that the negatively selectable marker is linked to the exogenous DNA, but configured such that the negatively selectable marker flanks the targeting sequence, and such that a correct homologous recombination event with sequences in the host cell genome does not result in the stable integration of the negatively selectable marker. Markers useful for this purpose include the Herpes Simplex Virus thymidine kinase (TK) gene or the bacterial xanthine-guanine phosphoribosyl-transferase (gpt) gene.

The gene targeting or gene activation techniques which can be used in accordance with this aspect of the invention are more particularly described in U.S. Pat. No. 5,272,071 to Chappel; U.S. Pat. No. 5,578,461 to Sherwin et al.; International Application No. PCT/US92/09627 (WO93/09222) by Selden et al.; and International Application No. PCT/US90/06436 (WO91/06667) by Skoultchi et al., each of which is incorporated by reference herein in its entirety.

5.5 Anti-KIRHy Antibodies

Alternatively, immunotargeting involves the administration of components of the immune system, such as antibodies, antibody fragments, or primed cells of the immune system against the target. Methods of immunotargeting cancer cells using antibodies or antibody fragments are well known in the art. U.S. Pat. No. 6,306,393 describes the use of anti-CD22 antibodies in the immunotherapy of B-cell malignancies, and U.S. Pat. No. 6,329,503 describes immunotargeting of cells that express serpentine transmembrane antigens (both U.S. patents are herein incorporated by reference in their entirety).

KIRHy antibodies (including humanized or human monoclonal antibodies or fragments or other modifications thereof, optionally conjugated to cytotoxic agents) may be introduced into a patient such that the antibody binds to KIRHy expressed by cancer cells and mediates the destruction of the cells and the tumor and/or inhibits the growth of the cells or the tumor. Without intending to limit the disclosure, mechanisms by which such antibodies can exert a therapeutic effect may include complement-mediated cytolysis, antibody-dependent cellular cytotoxicity (ADCC), modulating the physiologic function of KIRHy, inhibiting binding or signal transduction pathways, modulating tumor cell differentiation, altering tumor angiogenesis factor profiles, modulating the secretion of immune stimulating or tumor suppressing cytokines and growth factors, modulating cellular adhesion, and/or by inducing apoptosis. KIRHy antibodies conjugated to toxic or therapeutic agents, such as radioligands or cytosolic toxins, may also be used therapeutically to deliver the toxic or therapeutic agent directly to KIRHy-bearing tumor cells.

KIRHy antibodies may be used to suppress the immune system in patients receiving organ transplants or in patients with autoimmune diseases such as arthritis. Healthy immune cells would be targeted by these antibodies leading their death and clearance from the system, thus suppressing the immune system.

KIRHy antibodies may be used as antibody therapy for solid tumors which express this action. Cancer immunotherapy using antibodies provides a novel approach to treating cancers associated with cells that specifically express KIRHy. As described above, KIRHy is highly expressed in AML and histiocytic lymphoma cell lines indicating that KIRHy may be used as an therapeutic antibody target and a diagnostic marker for certain cell types or disorders (e.g., AML). Cancer immunotherapy using antibodies has been previously described for other types of cancer, including but not limited to colon cancer (Arlen et al., Crit. Rev. Immunol. 18:133-138 (1998)), multiple myeloma (Ozaki et al., Blood 90:3179-3186 (1997); Tsunenari et al., Blood 90:2437-2444 (1997)), gastric cancer (Kasprzyk et al., Cancer Res. 52:2771-2776 (1992)), B cell lymphoma (Funakoshi et al., J. Immunother. Emphasisi Tumor Immunol. 19:93-101 (1996)), leukemia (Zhong et al., Leuk. Res. 20:581-589 (1996)), colorectal cancer (Moun et al., Cancer Res. 54:6160-6166 (1994); Velders et al., Cancer Res. 55:4398-4403 (1995)), and breast cancer (Shepard et al., J. Clin. Immunol. 11:117-127 (1991), all of the above listed references are herein incorporated by reference in their entirety).

Although KIRHy antibody therapy may be useful for all stages of the foregoing cancers, antibody therapy may be particularly appropriate in advanced or metastatic cancers. Combining the antibody therapy method with a chemotherapeutic, radiation or surgical regimen may be preferred in patients that have not received chemotherapeutic treatment, whereas treatment with the antibody therapy may be indicated for patients who have received one or more chemotherapies. Additionally, antibody therapy can also enable the use of reduced dosages of concomitant chemotherapy, particularly in patients that do not tolerate the toxicity of the chemotherapeutic agent very well. Furthermore, treatment of cancer patients with KIRHy antibody with tumors resistant to chemotherapeutic agents might induce sensitivity and responsiveness to these agents in combination.

Prior to anti-KIRHy immunotargeting, a patient may be evaluated for the presence and level of KIRHy expression by the cancer cells, preferably using immunohistochemical assessments of tumor tissue, quantitative KIRHy imaging, quantitative RT-PCR, or other techniques capable of reliably indicating the presence and degree of KIRHy expression. For example, a blood or biopsy sample may be evaluated by immunohistochemical methods to determine the presence of KIRHy-expressing cells or to determine the extent of KIRHy expression on the surface of the cells within the sample. Methods for immunohistochemical analysis of tumor tissues or released fragments of KIRHy in the serum are well known in the art.

Anti-KIRHy antibodies useful in treating cancers include those, which are capable of initiating a potent immune response against the tumor and those, which are capable of direct cytotoxicity. In this regard, anti-KIRHy monoclonal antibodies may elicit tumor cell lysis by either complement-mediated or ADCC mechanisms, both of which require an intact Fc portion of the immunoglobulin molecule for interaction with effector cell Fc receptor sites or complement proteins. In addition, anti-KIRHy antibodies that exert a direct biological effect on tumor growth are useful in the practice of the invention. Potential mechanisms by which such directly cytotoxic antibodies may act include inhibition of cell growth, modulation of cellular differentiation, modulation of tumor angiogenesis factor profiles, and the induction of apoptosis. The mechanism by which a particular anti-KIRHy antibody exerts an anti-tumor effect may be evaluated using any number of in vitro assays designed to determine ADCC, ADMMC, complement-mediated cell lysis, and so forth, as is generally known in the art.

The anti-tumor activity of a particular anti-KIRHy antibody, or combination of anti-KIRHy antibody, may be evaluated in vivo using a suitable animal model, for example, xenogenic AML cancer models wherein human AML cells are introduced into immune compromised animals, such as nude or SCID mice. Efficacy may be predicted using assays, which measure inhibition of tumor formation, tumor regression or metastasis, and the like.

It should be noted that the use of murine or other non-human monoclonal antibodies, human/mouse chimeric mAbs may induce moderate to strong immune responses in some patients. In the most severe cases, such an immune response may lead to the extensive formation of immune complexes, which, potentially, can cause renal failure. Accordingly, preferred monoclonal antibodies used in the practice of the therapeutic methods of the invention are those which are either fully human or humanized and which bind specifically to the target KIRHy antigen with high affinity but exhibit low or no antigenicity in the patient.

The method of the invention contemplates the administration of single anti-KIRHy monoclonal antibodies (mAbs) as well as combinations, or “cocktails”, of different mAbs. Two or more monoclonal antibodies that bind to KIRHy may provide an improved effect compared to a single antibody. Alternatively, a combination of an anti-KIRHy antibody with an antibody that binds a different antigen may provide an improved effect compared to a single antibody. Such mAb cocktails may have certain advantages inasmuch as they contain mAbs, which exploit different effector mechanisms or combine directly cytotoxic mAbs with mAbs that rely on immune effector functionality. Such mAbs in combination may exhibit synergistic therapeutic effects. In addition, the administration of anti-KIRHy mAbs may be combined with other therapeutic agents, including but not limited to various chemotherapeutic agents, androgen-blockers, and immune modulators (e.g., IL-2, GM-CSF). The anti-KIRHy mAbs may be administered in their “naked” or unconjugated form, or may have therapeutic agents conjugated to them. Additionally, bispecific antibodies may be used. Such an antibody would have one antigenic binding domain specific for KIRHy and the other antigenic binding domain specific for another antigen (such as CD20 for example). Finally, Fab KIRHy antibodies or fragments of these antibodies (including fragments conjugated to other protein sequences or toxins) may also be used as therapeutic agents.

Antibodies that specifically bind KIRHy are useful in compositions and methods for immunotargeting cells expressing KIRHy and for diagnosing a disease or disorder wherein cells involved in the disorder express KIRHy. Such antibodies include monoclonal and polyclonal antibodies, single chain antibodies, chimeric antibodies, bifunctional/bispecific antibodies, humanized antibodies, human antibodies, and complementary determining region (CDR)-grafted antibodies, including compounds that include CDR and/or antigen-binding sequences, which specifically recognize KIRHy. Antibody fragments, including Fab, Fab′, F(ab′)₂, and Fᵥ, are also useful.

The term “specific for” indicates that the variable regions of the antibodies recognize and bind KIRHy exclusively (i.e., able to distinguish KIRHy from other similar polypeptides despite sequence identity, homology, or similarity found in the family of polypeptides), but may also interact with other proteins (for example, S. aureus protein A or other antibodies in ELISA techniques) through interactions with sequences outside the variable region of the antibodies, and in particular, in the constant region of the molecule. Screening assays in which one can determine binding specificity of an anti-KIRHy antibody are well known and routinely practiced in the art. (Chapter 6, Antibodies A Laboratory Manual, Eds. Harlow, et al., Cold Spring Harbor Laboratory; Cold Spring Harbor, N.Y. (1988), herein incorporated by reference in its entirety).

KIRHy polypeptides can be used to immunize animals to obtain polyclonal and monoclonal antibodies that specifically react with KIRHy. Such antibodies can be obtained using either the entire protein or fragments thereof as an immunogen. The peptide immunogens additionally may contain a cysteine residue at the carboxyl terminus, and are conjugated to a hapten such as keyhole limpet hemocyanin (KLH). Methods for synthesizing such peptides have been previously described (Merrifield, J. Amer. Chem. Soc. 85, 2149-2154 (1963); Krstenansky, et al., FEBS Lett. 211: 10 (1987), both of which are incorporated by reference in their entirety). Techniques for preparing polyclonal and monoclonal antibodies as well as hybridomas capable of producing the desired antibody have also been previously disclosed (Campbell, Monoclonal Antibodies Technology: Laboratory Techniques in Biochemistry and Molecular Biology, Elsevier Science Publishers, Amsterdam, The Netherlands (1984); St. Groth, et al., J. Immunol. 35:1-21 (1990); Kohler and Milstein, Nature 256:495-497 (1975)), the trioma technique, the human B-cell hybridoma technique (Kozbor, et al., Immunology Today 4:72 (1983); Cole, et al., in, Monoclonal Antibodies and Cancer Therapy, Alan R. Liss, Inc., pp. 77-96 (1985), all of which are incorporated by reference in their entirety).

Any animal capable of producing antibodies can be immunized with a KIRHy peptide or polypeptide. Methods for immunization include subcutaneous or intraperitoneal injection of the polypeptide. The amount of the KIRHy peptide or polypeptide used for immunization depends on the animal that is immunized, antigenicity of the peptide and the site of injection. The KIRHy peptide or polypeptide used as an immunogen may be modified or administered in an adjuvant in order to increase the protein's antigenicity. Methods of increasing the antigenicity of a protein are well known in the art and include, but are not limited to, coupling the antigen with a heterologous protein (such as globulin or β-galactosidase) or through the inclusion of an adjuvant during immunization.

For monoclonal antibodies, spleen cells from the immunized animals are removed, fused with myeloma cells, such as SP2/0-Ag14 myeloma cells, and allowed to become monoclonal antibody producing hybridoma cells. Any one of a number of methods well known in the art can be used to identify the hybridoma cell that produces an antibody with the desired characteristics. These include screening the hybridomas with an ELISA assay, Western blot analysis, or radioimmunoassay (Lutz, et al., Exp. Cell Res. 175:109-124 (1988), herein incorporated by reference in its entirety). Hybridomas secreting the desired antibodies are cloned and the class and subclass is determined using procedures known in the art (Campbell, A. M., Monoclonal Antibody Technology: Laboratory Techniques in Biochemistry and Molecular Biology, Elsevier Science Publishers, Amsterdam, The Netherlands (1984), herein incorporated by reference in its entirety). Techniques described for the production of single chain antibodies can be adapted to produce single chain antibodies to KIRHy (U.S. Pat. No. 4,946,778, herein incorporated by reference in its entirety).

For polyclonal antibodies, antibody-containing antiserum is isolated from the immunized animal and is screened for the presence of antibodies with the desired specificity using one of the above-described procedures.

Because antibodies from rodents tend to elicit strong immune responses against the antibodies when administered to a human, such antibodies may have limited effectiveness in therapeutic methods of the invention. Methods of producing antibodies that do not produce a strong immune response against the administered antibodies are well known in the art. For example, the anti-KIRHy antibody can be a nonhuman primate antibody. Methods of making such antibodies in baboons are disclosed in PCT publication No. WO 91/11465 and Losman et al., Int. J. Cancer 46:310-314 (1990), both of which are herein incorporated by reference in their entirety. In one embodiment, the anti-KIRHy antibody is a humanized monoclonal antibody. Methods of producing humanized antibodies have been previously described. (U.S. Pat. Nos. 5,997,867 and 5,985,279, Jones et al., Nature 321:522 (1986); Riechmann et al., Nature 332:323(1988); Verhoeyen et al., Science 239:1534-1536 (1988); Carter et al., Proc. Nat'l Acad. Sci. USA 89:4285-4289 (1992); Sandhu, Crit. Rev. Biotech. 12:437-462 (1992); and Singer, et al., J. Immun. 150:2844-2857 (1993), all of which are herein incorporated by reference in their entirety). In another embodiment, the anti-KIRHy antibody is a human monoclonal antibody. Humanized antibodies are produced by transgenic mice that have been engineered to produce human antibodies. Hybridomas derived from such mice will secrete large amounts of human monoclonal antibodies. Methods for obtaining human antibodies from transgenic mice are described in Green, et al., Nature Genet. 7:13-21(1994), Lonberg, et al., Nature 368:856 (1994), and Taylor, et al., Int. Immun. 6:579 (1994), all of which are herein incorporated by reference in their entirety.

The present invention also includes the use of anti-KIRHy antibody fragments. Antibody fragments can be prepared by proteolytic hydrolysis of an antibody or by expression in E. coli of the DNA coding for the fragment. Antibody fragments can be obtained by pepsin or papain digestion of whole antibodies. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab fragments and an Fc fragment directly. These methods have been previously described (U.S. Pat. Nos. 4,036,945 and 4,331,647, Nisonoff, et al., Arch Biochem. Biophys. 89:230 (1960); Porter, Biochem. J. 73:119 (1959), Edelman, et al., Meth. Enzymol. 1:422 (1967), all of which are herein incorporated by reference in their entirety). Other methods of cleaving antibodies, such as separation of heavy chains to form monovalent light-heavy chain fragments, further cleavage of fragments, or other enzymatic, chemical or genetic techniques may also be used, so long as the fragments bind to the antigen that is recognized by the intact antibody. For example, Fv fragments comprise an association of V_{H} and V_{L} chains, which can be noncovalent (Inbar et al., Proc. Nat'l Acad. Sci. USA 69:2659 (1972), herein incorporated by reference in its entirety). Alternatively, the variable chains can be linked by an intermolecular disulfide bond or cross-linked by chemicals such as glutaraldehyde.

In one embodiment, the Fv fragments comprise V_{H} and V_{L} chains that are connected by a peptide linker. These single-chain antigen binding proteins (sFv) are prepared by constructing a structural gene comprising DNA sequences encoding the V_{H} and V_{L} domains which are connected by an oligonucleotide. The structural gene is inserted into an expression vector, which is subsequently introduced into a host cell, such as E. coli. The recombinant host cells synthesize a single polypeptide chain with a linker peptide bridging the two V domains. Methods for producing sFvs have been previously described (U.S. Pat. No. 4,946,778, Whitlow, et al., Methods: A Companion to Methods in Enzymology 2:97 (1991), Bird, et al., Science 242:423 (1988), Pack, et al., Bio/Technology 11:1271 (1993), all of which are herein incorporated by reference in their entirety).

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (Larrick, et al., Methods: A Companion to Methods in Enzymology 2:106 (1991); Courtenay-Luck, pp. 166-179 in, Monoclonal Antibodies Production, Engineering and Clinical Applications, Eds. Ritter et al., Cambridge University Press (1995); Ward, et al., pp. 137-185 in, Monoclonal Antibodies Principles and Applications, Eds. Birch et al., Wiley-Liss, Inc. (1995), all of which are herein incorporated by reference in their entirety).

The present invention further provides the above-described antibodies in detectably labeled form. Antibodies can be detectably labeled through the use of radioisotopes, affinity labels (such as biotin, avidin, etc.), enzymatic labels (such as horseradish peroxidase, alkaline phosphatase, etc.) fluorescent labels (such as FITC or rhodamine, etc.), paramagnetic atoms, etc. Procedures for accomplishing such labeling have been previously disclosed (Sternberger, et al., J. Histochem. Cytochem. 18:315 (1970); Bayer, et al., Meth. Enzym. 62:308 (1979); Engval, et al., Immunol. 109:129 (1972); Goding, J. Immunol. Meth. 13:215 (1976), all of which are herein incorporated by reference in their entirety).

The labeled antibodies can be used for in vitro, in vivo, and in situ assays to identify cells or tissues in which KIRHy is expressed. Furthermore, the labeled antibodies can be used to identify the presence of secreted KIRHy in a biological sample, such as a blood, urine, lymphatic fluid, saliva and other extracellular fluid samples.

5.5.1 Anti-KIRHy Antibody Conjugates

The present invention contemplates the use of “naked” anti-KIRHy antibodies, as well as the use of immunoconjugates. Immunoconjugates can be prepared by indirectly conjugating a therapeutic agent such as a cytotoxic agent to an antibody component. Toxic moieties include, for example, plant toxins, such as abrin, ricin, modeccin, viscumin, pokeweed anti-viral protein, saporin, gelonin, momoridin, trichosanthin, barley toxin; bacterial toxins, such as Diptheria toxin, Pseudomonas endotoxin and exotoxin, Staphylococcal enterotoxin A; fungal toxins, such as α-sarcin, restrictocin; cytotoxic RNases, such as extracellular pancreatic RNases; DNase I (Pastan, et al., Cell 47:641 (1986); Goldenberg, Cancer Journal for Clinicians 44:43 (1994), herein incorporated by reference in their entirety), calicheamicin, and radioisotopes, such as ³²P, ⁶⁷Cu, ⁷⁷As, ¹⁰⁵Rh, ¹⁰⁹Pd, ¹¹¹Ag, ¹²¹Sn, ¹³¹I, ¹⁶⁶Ho, ¹⁷⁷Lu, ¹⁸⁶Re, ¹⁸⁸Re, ¹⁹⁴Ir, ¹⁹⁹Au (Illidge and Brock, Curr Pharm. Design 6:1399 (2000), herein incorporated by reference in its entirety). In humans, clinical trials are underway utilizing a yttrium-90 conjugated anti-CD20 antibody for B cell lymphomas (Cancer Chemother Pharmacol 48(Suppl 1):S91-S95 (2001), herein incorporated by reference in its entirety).

General techniques have been previously described (U.S. Pat. Nos. 6,306,393 and 5,057,313, Shih, et al., Int. J. Cancer 41:832-839 (1988); Shih, et al., Int. J. Cancer 46:1101-1106 (1990), all of which are herein incorporated by reference in their entirety). The general method involves reacting an antibody component having an oxidized carbohydrate portion with a carrier polymer that has at least one free amine function and that is loaded with a plurality of drug, toxin, chelator, boron addends, or other therapeutic agent. This reaction results in an initial Schiff base (imine) linkage, which can be stabilized by reduction to a secondary amine to form the final conjugate.

The carrier polymer is preferably an aminodextran or polypeptide of at least 50 amino acid residues, although other substantially equivalent polymer carriers can also be used. Preferably, the final immunoconjugate is soluble in an aqueous solution, such as mammalian serum, for ease of administration and effective targeting for use in therapy. Thus, solubilizing functions on the carrier polymer will enhance the serum solubility of the final immunoconjugate. In particular, an aminodextran will be preferred.

The process for preparing an immunoconjugate with an aminodextran carrier typically begins with a dextran polymer, advantageously a dextran of average molecular weight of about 10,000-100,000. The dextran is reacted with an oxidizing agent to affect a controlled oxidation of a portion of its carbohydrate rings to generate aldehyde groups. The oxidation is conveniently effected with glycolytic chemical reagents such as NaIO₄, according to conventional procedures. The oxidized dextran is then reacted with a polyamine, preferably a diamine, and more preferably, a mono- or polyhydroxy-diamine. Suitable amines include ethylene diamine, propylene diamine, or other like polymethylene diamines, diethylene triamine or like polyamines, 1,3-diamino-2-hydroxypropane, or other like hydroxylated diamines or polyamines, and the like. An excess of the amine relative to the aldehyde groups of the dextran is used to ensure substantially complete conversion of the aldehyde functions to Schiff base groups. A reducing agent, such as NaBH₄, NaBH₃ CN or the like, is used to effect reductive stabilization of the resultant Schiff base intermediate. The resultant adduct can be purified by passage through a conventional sizing column or ultrafiltration membrane to remove cross-linked dextrans. Other conventional methods of derivatizing a dextran to introduce amine functions can also be used, e.g., reaction with cyanogen bromide, followed by reaction with a diamine.

The aminodextran is then reacted with a derivative of the particular drug, toxin, chelator, immunomodulator, boron addend, or other therapeutic agent to be loaded, in an activated form, preferably, a carboxyl-activated derivative, prepared by conventional means, e.g., using dicyclohexylcarbodiimide (DCC) or a water soluble variant thereof, to form an intermediate adduct. Alternatively, polypeptide toxins such as pokeweed antiviral protein or ricin A-chain, and the like, can be coupled to aminodextran by glutaraldehyde condensation or by reaction of activated carboxyl groups on the protein with amines on the aminodextran.

Chelators for radiometals or magnetic resonance enhancers are well-known in the art. Typical are derivatives of ethylenediaminetetraacetic acid (EDTA) and diethylenetriaminepentaacetic acid (DTPA). These chelators typically have groups on the side chain by which the chelator can be attached to a carrier. Such groups include, e.g., benzylisothiocyanate, by which the DTPA or EDTA can be coupled to the amine group of a carrier. Alternatively, carboxyl groups or amine groups on a chelator can be coupled to a carrier by activation or prior derivatization and then coupling, all by well-known means.

Boron addends, such as carboranes, can be attached to antibody components by conventional methods. For example, carboranes can be prepared with carboxyl functions on pendant side chains, as is well known in the art. Attachment of such carboranes to a carrier, e.g., aminodextran, can be achieved by activation of the carboxyl groups of the carboranes and condensation with amines on the carrier to produce an intermediate conjugate. Such intermediate conjugates are then attached to antibody components to produce therapeutically useful immunoconjugates, as described below.

A polypeptide carrier can be used instead of aminodextran, but the polypeptide carrier should have at least 50 amino acid residues in the chain, preferably 100-5000 amino acid residues. At least some of the amino acids should be lysine residues or glutamate or aspartate residues. The pendant amines of lysine residues and pendant carboxylates of glutamine and aspartate are convenient for attaching a drug, toxin, immunomodulator, chelator, boron addend or other therapeutic agent. Examples of suitable polypeptide carriers include polylysine, polyglutamic acid, polyaspartic acid, co-polymers thereof, and mixed polymers of these amino acids and others, e.g., serines, to confer desirable solubility properties on the resultant loaded carrier and immunoconjugate.

Conjugation of the intermediate conjugate with the antibody component is effected by oxidizing the carbohydrate portion of the antibody component and reacting the resulting aldehyde (and ketone) carbonyls with amine groups remaining on the carrier after loading with a drug, toxin, chelator, immunomodulator, boron addend, or other therapeutic agent. Alternatively, an intermediate conjugate can be attached to an oxidized antibody component via amine groups that have been introduced in the intermediate conjugate after loading with the therapeutic agent. Oxidation is conveniently effected either chemically, e.g., with NaIO₄ or other glycolytic reagent, or enzymatically, e.g., with neuraminidase and galactose oxidase. In the case of an aminodextran carrier, not all of the amines of the aminodextran are typically used for loading a therapeutic agent. The remaining amines of aminodextran condense with the oxidized antibody component to form Schiff base adducts, which are then reductively stabilized, normally with a borohydride reducing agent.

Analogous procedures are used to produce other immunoconjugates according to the invention. Loaded polypeptide carriers preferably have free lysine residues remaining for condensation with the oxidized carbohydrate portion of an antibody component. Carboxyls on the polypeptide carrier can, if necessary, be converted to amines by, e.g., activation with DCC and reaction with an excess of a diamine.

The final immunoconjugate is purified using conventional techniques, such as sizing chromatography on Sephacryl S-300 or affinity chromatography using one or more KIRHy epitopes.

Alternatively, immunoconjugates can be prepared by directly conjugating an antibody component with a therapeutic agent. The general procedure is analogous to the indirect method of conjugation except that a therapeutic agent is directly attached to an oxidized antibody component. It will be appreciated that other therapeutic agents can be substituted for the chelators described herein. Those of skill in the art will be able to devise conjugation schemes without undue experimentation.

As a further illustration, a therapeutic agent can be attached at the hinge region of a reduced antibody component via disulfide bond formation. For example, the tetanus toxoid peptides can be constructed with a single cysteine residue that is used to attach the peptide to an antibody component. As an alternative, such peptides can be attached to the antibody component using a heterobifunctional cross-linker, such as N-succinyl 3-(2-pyridyldithio)proprionate (SPDP) (Yu, et al., Int. J. Cancer 56:244 (1994), herein incorporated by reference in its entirety). General techniques for such conjugation have been previously described (Wong, Chemistry of Protein Conjugation and Cross-linking, CRC Press (1991); Upeslacis, et al., pp. 187-230 in, Monoclonal Antibodies Principles and Applications, Eds. Birch et al., Wiley-Liss, Inc. (1995); Price, pp. 60-84 in, Monoclonal Antibodies: Production, Engineering and Clinical Applications Eds. Ritter, et al., Cambridge University Press (1995), all of which are herein incorporated by reference in their entirety).

As described above, carbohydrate moieties in the Fc region of an antibody can be used to conjugate a therapeutic agent. However, the Fc region may be absent if an antibody fragment is used as the antibody component of the immunoconjugate. Nevertheless, it is possible to introduce a carbohydrate moiety into the light chain variable region of an antibody or antibody fragment (Leung, et al., J. Immunol. 154:5919-5926 (1995); U.S. Pat. No. 5,443,953), both of which are herein incorporated by reference in their entirety. The engineered carbohydrate moiety is then used to attach a therapeutic agent.

In addition, those of skill in the art will recognize numerous possible variations of the conjugation methods. For example, the carbohydrate moiety can be used to attach polyethyleneglycol in order to extend the half-life of an intact antibody, or antigen-binding fragment thereof, in blood, lymph, or other extracellular fluids. Moreover, it is possible to construct a “divalent immunoconjugate” by attaching therapeutic agents to a carbohydrate moiety and to a free sulfhydryl group. Such a free sulfhydryl group may be located in the hinge region of the antibody component.

5.5.2 Anti-KIRHy Antibody Fusion Proteins

When the therapeutic agent to be conjugated to the antibody is a protein, the present invention contemplates the use of fusion proteins comprising one or more anti-KIRHy antibody moieties and an immunomodulator or toxin moiety. Methods of making antibody fusion proteins have been previously described (U.S. Pat. No. 6,306,393, herein incorporated by reference in its entirety). Antibody fusion proteins comprising an interleukin-2 moiety have also been previously disclosed (Boleti, et al., Ann. Oncol. 6:945 (1995), Nicolet, et al., Cancer Gene Ther. 2:161 (1995), Becker, et al., Proc. Nat'l Acad. Sci. USA 93:7826 (1996), Hank, et al., Clin. Cancer Res. 2:1951 (1996), Hu, et al., Cancer Res. 56:4998 (1996) all of which are herein incorporated by reference in their entirety). In addition, Yang, et al., Hum. Antibodies Hybridomas 6:129 (1995), herein incorporated by reference in its entirety, describe a fusion protein that includes an F(ab′)₂ fragment and a tumor necrosis factor alpha moiety.

Methods of making antibody-toxin fusion proteins in which a recombinant molecule comprises one or more antibody components and a toxin or chemotherapeutic agent also are known to those of skill in the art. For example, antibody-Pseudomonas exotoxin A fusion proteins have been described (Chaudhary, et al., Nature 339:394 (1989), Brinkmann, et al., Proc. Nat'l Acad. Sci. USA 88:8616 (1991), Batra, et al., Proc. Natl. Acad. Sci. USA 89:5867 (1992), Friedman, et al., J. Immunol. 150:3054 (1993), Wels, et al., Int. J. Can. 60:137 (1995), Fominaya et al., J. Biol. Chem. 271:10560 (1996), Kuan, et al., Biochemistry 35:2872 (1996), Schmidt, et al., Int. J. Can. 65:538 (1996), all of which are herein incorporated by reference in their entirety). Antibody-toxin fusion proteins containing a diphtheria toxin moiety have been described (Kreitman, et al., Leukemia 7:553 (1993), Nicholls, et al., J. Biol. Chem. 268:5302 (1993), Thompson, et al., J. Biol. Chem. 270:28037 (1995), and Vallera, et al., Blood 88:2342 (1996). Deonarain et al. (Tumor Targeting 1:177 (1995)), have described an antibody-toxin fusion protein having an RNase moiety, while Linardou, et al. (Cell Biophys. 24-25:243 (1994), all of which are herein incorporated by reference in their entirety), produced an antibody-toxin fusion protein comprising a DNase I component. Gelonin and Staphylococcal enterotoxin-A have been used as the toxin moieties in antibody-toxin fusion proteins (Wang, et al., Abstracts of the 209th ACS National Meeting, Anaheim, Calif., Apr. 2-6, 1995, Part 1, BIOT005; Dohlsten, et al., Proc. Nat'l Acad. Sci. USA 91:8945 (1994), both of which herein incorporated by reference in their entirety).

5.5.3 Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface-active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants that can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

5.5.4 Monoclonal Antibodies

The term “monoclonal antibody” (mAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen-binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368:812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

5.5.5 Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann, et al., Nature, 332:323-327 (1988); Verhoeyen, et al., Science, 239:1534-1536 (1988)), by substituting rodent CDRs or CDR sequences for the corresponding sequences of a human antibody. (See also U.S. Pat. No. 5,225,539). In some instances, Fv framework residues of the human immunoglobulin are replaced by corresponding non-human residues. Humanized antibodies can also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

5.5.6 Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., Immunol Today 4: 72 (1983)) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., Proc Natl Acad Sci USA 80: 2026-2030 (1983)) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10:779-783 (1992)); Lonberg et al. (Nature 368:856-859 (1994)); Morrison (Nature 368:812-13 (1994)); Fishwild et al, (Nature Biotechnology, 14:845-51 (1996)); Neuberger (Nature Biotechnology, 14:826 (1996)); and Lonberg and Huszar (Intern. Rev. Immunol. 13:65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

5.5.7 FAB Fragments and Single Chain KIRHy Antibodies

According to the invention, techniques can be adapted for the production of single-chain antibodies specific to KIRHy (see e.g., U.S. Pat. No. 4,946,778). In addition, methods can be adapted for the construction of F_{ab} expression libraries (see e.g., Huse, et al., Science 246:1275-1281 (1989)) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

5.5.8 Bispecific KIRHy Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., 1991 EMBO J., 10, 3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121: 210 (1986).

According to another approach described in WO 96/27011, the interface between a pair of antibody molecules can be engineered to maximize the percentage of heterodimers that are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full-length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148:1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

5.5.9 Heteroconjugate KIRHy Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

5.5.10 Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176:1191-1195 (1992) and Shopes, J. Immunol., 148:2918-2922 (1992). Homodimeric antibodies with enhanced anti-tumor activity can also be prepared using heterobifunctional cross-linkers as described in Wolff et al. Cancer Research, 53:2560-2565 (1993). Alternatively, an antibody can be engineered that has dual Fc regions and can thereby have enhanced complement lysis and ADCC capabilities. See Stevenson et al., Anti-Cancer Drug Design, 3:219-230 (1989).

5.5.11 Generating Antibodies Using Phage Display

The antibodies of the present invention can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous-phage including fd and M13 binding domains expressed from phage with Fab, Fv or disulfide-stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187:9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax et al., BioTechniques 12(6):864-869 (1992); and Sawai et al., AJRI 34:2634 (1995); and Better et al., Science 240:1041-1043 (1988) (said references incorporated by reference in their entireties). Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:10381040 (1988).

5.5.12 Polynucleotides Encoding Antibodies

The invention further provides polynucleotides comprising a nucleotide sequence encoding an antibody of the invention and fragments thereof. The invention also encompasses polynucleotides that hybridize under stringent or lower stringency hybridization conditions, e.g., as defined supra, to polynucleotides that encode an antibody, preferably, that specifically binds to a polypeptide of the invention, preferably, an antibody that binds to a polypeptide having the amino acid sequence of any one of SEQ ID NO: 3-7, 11, 13, 15, 17, 19, 21, 23, 25-29, 31-37, 39, 41, 43, 45, 47, 49, 51.

The polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994) herein incorporated by reference in its entirety), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence and corresponding amino acid sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

In a specific embodiment, the amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody, as described supra. The framework regions may be naturally occurring or consensus framework regions, and preferably human framework regions (see, e.g., Chothia et al., J. Mol. Biol. 278: 457-479 (1998) for a listing of human framework regions (herein incorporated by reference in its entirety)). Preferably, the polynucleotide generated by the combination of the framework regions and CDRs encodes an antibody that specifically binds a polypeptide of the invention. Preferably, as discussed supra, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985), all of which are herein incorporated by reference in their entirety) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human immunoglobulin constant region, e.g., humanized antibodies.

Alternatively, techniques described for the production of single chain antibodies (U.S. Pat. No. 4,946,778; Bird, Science 242:423-442 (1988); Huston et al., Proc. Natl. Acad. Sci. USA 85:5879-5883 (1988); and Ward et al., Nature 334:544-54 (1989) all of which are herein incorporated by reference in their entirety) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science 242:1038-1041 (1988) herein incorporated by reference in its entirety).

More preferably, a clone encoding an antibody of the present invention may be obtained according to the method described in the Example section herein.

5.5.13 Antibody-Based Gene Therapy

In a specific embodiment, nucleic acids comprising sequences encoding antibodies or functional derivatives thereof, are administered to treat, inhibit or prevent a disease or disorder associated with aberrant expression and/or activity of a polypeptide of the invention, by way of gene therapy. Gene therapy refers to therapy performed by the administration to a subject of an expressed or expressible nucleic acid. In this embodiment of the invention, the nucleic acids produce their encoded protein that mediates a therapeutic effect.

Any of the methods for gene therapy available in the art can be used according to the present invention. Exemplary methods are described below.

For general reviews of the methods of gene therapy, see Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 11(5):155-215 (1993) (all of which are each herein incorporated by reference in their entirety). Methods commonly known in the art of recombinant DNA technology which can be used are described in Ausubel et al. (eds.), Current Protocols in Molecular Biology, John Wiley & Sons, NY (1993); and 30 Kriegler, Gene Transfer and Expression, A Laboratory Manual, Stockton Press, NY (1990).

In a preferred aspect, the compound comprises nucleic acid sequences encoding an antibody, said nucleic acid sequences being part of expression vectors that express the antibody or fragments or chimeric proteins or heavy or light chains thereof in a suitable host. In particular, such nucleic acid sequences have promoters operably linked to the antibody coding region, said promoter being inducible or constitutive, and, optionally, tissue-specific. In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, Proc. Natl. Acad. Sci. USA 86:8932-8935 (1989); Zijlstra et al., Nature 342:435-438 (1989). In specific embodiments, the expressed antibody molecule is a single chain antibody; alternatively, the nucleic acid sequences include sequences encoding both the heavy and light chains, or fragments thereof, of the antibody.

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem. 262:4429-4432 (1987)) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; W092/20316; W093/14188, WO 93/20221). Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijlstra et al., Nature 342:435-438 (1989) each of which is herein incorporated by reference in its entirety).

In a specific embodiment, viral vectors that contains nucleic acid sequences encoding an antibody of the invention are used. For example, a retroviral vector can be used (see Miller et al., Meth. Enzymol. 217:581-599 (1993) herein incorporated by reference in its entirety). These retroviral vectors contain the components necessary for the correct packaging of the viral genome and integration into the host cell DNA. The nucleic acid sequences encoding the antibody to be used in gene therapy are cloned into one or more vectors, which facilitates delivery of the gene into a patient. More detail about retroviral vectors can be found in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993), all of which are each herein incorporated by reference in its entirety.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). In a preferred embodiment, adenovirus vectors are used.

Adeno-associated virus (AAV) has also been proposed for use in gene therapy (Walsh et al., Proc, Soc. Exp. Biol. Med. 204:289-300 (1993); U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, Meth. Enzymol. 217:599-618 (1993); Cohen et al., Meth. Enzymol. 217:618-644 (1993); Cline, Pharmac. Ther. 29:69-92m (1985) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; 35 blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, nucleic acid sequences encoding an antibody are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g. PCT Publication WO 94/08598; Stemple and Anderson, Cell 71:973-985 (1992); Rheinwals, Meth. Cell Bio. 21A:229 (1980); and Pittelkow and Scott, May Clinic Proc. 61:771 (1986), all of which are herein incorporated by reference in their entirety).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

5.6 KIRHy Polypeptides

The isolated polypeptides of the invention include, but are not limited to, a polypeptide comprising: the amino acid sequence set forth as any one of SEQ ID NO: 3-7, 11, 13, 15, 17, 19, 21, 23, 25-29, 31-37, 39, 41, 43, 45, 47, 49, 51, or an amino acid sequence encoded by any one of the nucleotide sequences SEQ ID NO: 1-2, 12, 14, 16, 18, 20, 22, 24, 30, 38, 40, 42, 44, 46, 48, 50 or the corresponding full length or mature protein. Polypeptides of the invention also include polypeptides preferably with biological or immunological activity that are encoded by: (a) a polynucleotide having any one of the nucleotide sequences set forth in the SEQ ID NO: 1-2, 12, 14, 16, 18, 20, 22, 24, 30, 38, 40, 42, 44, 46, 48, 50 or (b) polynucleotides encoding any one of the amino acid sequences set forth as SEQ ID NO: 3-7, 11, 13, 15, 17, 19, 21, 23, 25-29, 31-37, 39, 41, 43, 45, 47, 49, 51, or (c) polynucleotides that hybridize to the complement of the polynucleotides of either (a) or (b) under stringent hybridization conditions. The invention also provides biologically active or immunologically active variants of any of the polypeptide amino acid sequences set forth as SEQ ID NO: 3-7, 11, 13, 15, 17, 19, 21, 23, 25-29, 31-37, 39, 41, 43, 45, 47, 49, 51, or the corresponding full length or mature protein; and “substantial equivalents” thereof (e.g., with at least about 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98% or 99%, most typically at least 99% amino acid identity) that retain biological activity. Polypeptides encoded by allelic variants may have a similar, increased, or decreased activity compared to polypeptides comprising SEQ ID NO: 3-7, 11, 13, 15, 17, 19, 21, 23, 25-29, 31-37, 39, 41, 43, 45, 47, 49, 51.

Fragments of the proteins of the present invention which are capable of exhibiting biological activity are also encompassed by the present invention. Fragments of the protein may be in linear form or they may be cyclized using known methods, for example, as described in H. U. Saragovi, et al., Bio/Technology 10:773-778 (1992) and in R. S. McDowell, et al., J. Amer. Chem. Soc. 114:9245-9253 (1992), both of which are incorporated herein by reference. Such fragments may be fused to carrier molecules such as immunoglobulins for many purposes, including increasing the valency of protein binding sites.

The present invention also provides both full-length and mature forms (for example, without a signal sequence or precursor sequence) of the disclosed proteins. The protein coding sequence is identified in the sequence listing by translation of the disclosed nucleotide sequences. The mature form of such protein may be obtained by expression of a full-length polynucleotide in a suitable mammalian cell or other host cell. The sequence of the mature form of the protein is also determinable from the amino acid sequence of the full-length form. Where proteins of the present invention are membrane bound, soluble forms of the proteins are also provided. In such forms, part or all of the regions causing the proteins to be membrane bound are deleted so that the proteins are fully secreted from the cell in which it is expressed.

Protein compositions of the present invention may further comprise an acceptable carrier, such as a hydrophilic, e.g., pharmaceutically acceptable, carrier.

The present invention further provides isolated polypeptides encoded by the nucleic acid fragments of the present invention or by degenerate variants of the nucleic acid fragments of the present invention. By “degenerate variant” is intended nucleotide fragments which differ from a nucleic acid fragment of the present invention (e.g., an ORF) by nucleotide sequence but, due to the degeneracy of the genetic code, encode an identical polypeptide sequence. Preferred nucleic acid fragments of the present invention are the ORFs that encode proteins.

A variety of methodologies known in the art can be utilized to obtain any one of the isolated polypeptides or proteins of the present invention. At the simplest level, the amino acid sequence can be synthesized using commercially available peptide synthesizers. The synthetically-constructed protein sequences, by virtue of sharing primary, secondary or tertiary structural and/or conformational characteristics with proteins may possess biological properties in common therewith, including protein activity. This technique is particularly useful in producing small peptides and fragments of larger polypeptides. Fragments are useful, for example, in generating antibodies against the native polypeptide. Thus, they may be employed as biologically active or immunological substitutes for natural, purified proteins in screening of therapeutic compounds and in immunological processes for the development of antibodies.

The polypeptides and proteins of the present invention can alternatively be purified from cells which have been altered to express the desired polypeptide or protein. As used herein, a cell is said to be altered to express a desired polypeptide or protein when the cell, through genetic manipulation, is made to produce a polypeptide or protein which it normally does not produce or which the cell normally produces at a lower level. One skilled in the art can readily adapt procedures for introducing and expressing either recombinant or synthetic sequences into eukaryotic or prokaryotic cells in order to generate a cell which produces one of the polypeptides or proteins of the present invention.

The invention also relates to methods for producing a polypeptide comprising growing a culture of host cells of the invention in a suitable culture medium, and purifying the protein from the cells or the culture in which the cells are grown. For example, the methods of the invention include a process for producing a polypeptide in which a host cell containing a suitable expression vector that includes a polynucleotide of the invention is cultured under conditions that allow expression of the encoded polypeptide. The polypeptide can be recovered from the culture, conveniently from the culture medium, or from a lysate prepared from the host cells and further purified. Preferred embodiments include those in which the protein produced by such process is a full length or mature form of the protein.

In an alternative method, the polypeptide or protein is purified from bacterial cells which naturally produce the polypeptide or protein. One skilled in the art can readily follow known methods for isolating polypeptides and proteins in order to obtain one of the isolated polypeptides or proteins of the present invention. These include, but are not limited to, immunochromatography, HPLC, size-exclusion chromatography, ion-exchange chromatography, and immuno-affinity chromatography. See, e.g., Scopes, Protein Purification Principles and Practice, Springer-Verlag (1994); Sambrook, et al., in Molecular Cloning: A Laboratory Manual; Ausubel et al., Current Protocols in Molecular Biology. Polypeptide fragments that retain biological/immunological activity include fragments comprising greater than about 100 amino acids, or greater than about 200 amino acids, and fragments that encode specific protein domains.

The purified polypeptides can be used in in vitro binding assays which are well known in the art to identify molecules which bind to the polypeptides. These molecules include but are not limited to, for e.g., small molecules, molecules from combinatorial libraries, antibodies or other proteins. The molecules identified in the binding assay are then tested for antagonist or agonist activity in in vivo tissue culture or animal models that are well known in the art. In brief, the molecules are titrated into a plurality of cell cultures or animals and then tested for either cell/animal death or prolonged survival of the animal/cells.

In addition, the peptides of the invention or molecules capable of binding to the peptides may be complexed with toxins, e.g., ricin or cholera, or with other compounds that are toxic to cells. The toxin-binding molecule complex is then targeted to a tumor or other cell by the specificity of the binding molecule for SEQ ID NO: 3-7, 11, 13, 15, 17, 19, 21, 23, 25-29, 31-37, 39, 41, 43, 45, 47, 49, 51.

The protein of the invention may also be expressed as a product of transgenic animals, e.g., as a component of the milk of transgenic cows, goats, pigs, or sheep which are characterized by somatic or germ cells containing a nucleotide sequence encoding the protein.

The proteins provided herein also include proteins characterized by amino acid sequences similar to those of purified proteins but into which modification are naturally provided or deliberately engineered. For example, modifications, in the peptide or DNA sequence, can be made by those skilled in the art using known techniques. Modifications of interest in the protein sequences may include the alteration, substitution, replacement, insertion or deletion of a selected amino acid residue in the coding sequence. For example, one or more of the cysteine residues may be deleted or replaced with another amino acid to alter the conformation of the molecule. Techniques for such alteration, substitution, replacement, insertion or deletion are well known to those skilled in the art (see, e.g., U.S. Pat. No. 4,518,584). Preferably, such alteration, substitution, replacement, insertion or deletion retains the desired activity of the protein. Regions of the protein that are important for the protein function can be determined by various methods known in the art including the alanine-scanning method which involved systematic substitution of single or strings of amino acids with alanine, followed by testing the resulting alanine-containing variant for biological activity. This type of analysis determines the importance of the substituted amino acid(s) in biological activity. Regions of the protein that are important for protein function may be determined by the eMATRIX program.

Other fragments and derivatives of the sequences of proteins which would be expected to retain protein activity in whole or in part and are useful for screening or other immunological methodologies may also be easily made by those skilled in the art given the disclosures herein. Such modifications are encompassed by the present invention.

The protein may also be produced by operably linking the isolated polynucleotide of the invention to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBat™ kit), and such methods are well known in the art, as described in Summers and Smith, Texas Agricultural Experiment Station Bulletin No. 1555 (1987), incorporated herein by reference. As used herein, an insect cell capable of expressing a polynucleotide of the present invention is “transformed.”

The protein of the invention may be prepared by culturing transformed host cells under culture conditions suitable to express the recombinant protein. The resulting expressed protein may then be purified from such culture (i.e., from culture medium or cell extracts) using known purification processes, such as gel filtration and ion exchange chromatography. The purification of the protein may also include an affinity column containing agents which will bind to the protein; one or more column steps over such affinity resins as concanavalin A-agarose, heparin-toyopearl™ or Cibacrom blue 3GA Sepharose™; one or more steps involving hydrophobic interaction chromatography using such resins as phenyl ether, butyl ether, or propyl ether; or immunoaffinity chromatography.

Alternatively, the protein of the invention may also be expressed in a form which will facilitate purification. For example, it may be expressed as a fusion protein, such as those of maltose binding protein (MBP), glutathione-S-transferase (GST) or thioredoxin (TRX), or as a His tag. Kits for expression and purification of such fusion proteins are commercially available from New England BioLab (Beverly, Mass.), Pharmacia (Piscataway, N.J.) and Invitrogen, respectively. The protein can also be tagged with an epitope and subsequently purified by using a specific antibody directed to such epitope. One such epitope (“FLAG®”) is commercially available from Kodak (New Haven, Conn.).

Finally, one or more reverse-phase high performance liquid chromatography (RP-HPLC) steps employing hydrophobic RP-HPLC media, e.g., silica gel having pendant methyl or other aliphatic groups, can be employed to further purify the protein. Some or all of the foregoing purification steps, in various combinations, can also be employed to provide a substantially homogeneous isolated recombinant protein. The protein thus purified is substantially free of other mammalian proteins and is defined in accordance with the present invention as an “isolated protein.”

The polypeptides of the invention include analogs (variants). The polypeptides of the invention include KIRHy analogs. This embraces fragments of KIRHy, as well KIRHy polypeptides which comprise one or more amino acids deleted, inserted, or substituted. Also, analogs of KIRHy embrace fusions of the KIRHy polypeptides or modifications of the KIRHy polypeptides, wherein the KIRHy polypeptide or analog is fused to another moiety or moieties, e.g., targeting moiety or another therapeutic agent. Such analogs may exhibit improved properties such as activity and/or stability. Examples of moieties which may be fused to the KIRHy polypeptide or an analog include, for example, targeting moieties which provide for the delivery of polypeptide to hematopoietic or cancer cells.

5.6.1 Hematopoiesis Regulating Activity of KIRHy

KIRHy may be involved in regulation of hematopoiesis and, consequently, in the treatment of myeloid or lymphoid cell disorders. Even marginal biological activity in support of colony forming cells or of factor-dependent cell lines indicates involvement in regulating hematopoiesis, e.g. in supporting the growth and proliferation of erythroid progenitor cells alone or in combination with other cytokines, thereby indicating utility, for example, in treating various anemias or for use in conjunction with irradiation/chemotherapy to stimulate the production of erythroid precursors and/or erythroid cells; in supporting the growth and proliferation of myeloid cells such as granulocytes and monocytes/macrophages (i.e., traditional CSF activity) useful, for example, in conjunction with chemotherapy to prevent or treat consequent myelo-suppression; in supporting the growth and proliferation of megakaryocytes and consequently of platelets thereby allowing prevention or treatment of various platelet disorders such as thrombocytopenia, and generally for use in place of or complimentary to platelet transfusions; and/or in supporting the growth and proliferation of hematopoietic stem cells which are capable of maturing to any and all of the above-mentioned hematopoietic cells and therefore find therapeutic utility in various stem cell disorders (such as those usually treated with transplantation, including, without limitation, aplastic anemia and paroxysmal nocturnal hemoglobinuria), as well as in repopulating the stem cell compartment post irradiation/chemotherapy, either in vivo or ex vivo (i.e., in conjunction with bone marrow transplantation or with peripheral progenitor cell transplantation (homologous or heterologous)) as normal cells or genetically manipulated for gene therapy.

Therapeutic compositions of the invention can be used in the following:

Suitable assays for proliferation and differentiation of various hematopoietic lines are cited above.

Assays for embryonic stem cell differentiation (which will identify, among others, proteins that influence embryonic differentiation hematopoiesis) include, without limitation, those described in: Johansson et al. Cell. Biol. 15:141-151, 1995; Keller et al., Mol. and Cell. Biol. 13:473-486, 1993; McClanahan et al., Blood 81:2903-2915, 1993.

Assays for stem cell survival and differentiation (which will identify, among others, proteins that regulate lympho-hematopoiesis) include, without limitation, those described in: Methylcellulose colony forming assays, Freshney, M. G. In Culture of Hematopoietic Cells. R. I. Freshney, et al. eds. Vol pp. 265-268, Wiley-Liss, Inc., New York, N.Y. 1994; Hirayama et al., Proc. Natl. Acad. Sci. USA 89:5907-5911, 1992; Primitive hematopoietic colony forming cells with high proliferative potential, McNiece, I. K. and Briddell, R. A. In Culture of Hematopoietic Cells. R. I. Freshney, et al. eds. Vol pp. 23-39, Wiley-Liss, Inc., New York, N.Y. 1994; Neben et al., Exp. Hematol. 22:353-359, 1994; Cobblestone area forming cell assay, Ploemacher, R. E. In Culture of Hematopoietic Cells. R. I. Freshney, et al. eds. Vol pp. 1-21, Wiley-Liss, Inc., New York, N.Y. 1994; Long term bone marrow cultures in the presence of stromal cells, Spooncer, E., Dexter, M. and Allen, T. In Culture of Hematopoietic Cells. R. I. Freshney, et al. eds. Vol pp. 163-179, Wiley-Liss, Inc., New York, N.Y. 1994; Long term culture initiating cell assay, Sutherland, H. J. In Culture of Hematopoietic Cells. R. I. Freshney, et al. eds. Vol pp. 139-162, Wiley-Liss, Inc., New York, N.Y. 1994.

5.6.2 Immune Stimulating or Suppressing Activity OF KIRHy

KIRHy may also exhibit immune stimulating or immune suppressing activity, including without limitation the activities for which assays are described herein. A polynucleotide of the invention can encode a polypeptide exhibiting such activities. A protein may be useful in the treatment of various immune deficiencies and disorders (including severe combined immunodeficiency (SCID)), e.g., in regulating (up or down) growth and proliferation of T and/or B lymphocytes, as well as effecting the cytolytic activity of NK cells and other cell populations. These immune deficiencies may be genetic or be caused by viral (e.g., HIV) as well as bacterial or fungal infections, or may result from autoimmune disorders. More specifically, infectious diseases causes by viral, bacterial, fungal or other infection may be treatable using a protein of the present invention, including infections by HIV, hepatitis viruses, herpes viruses, mycobacteria, Leishmania spp., malaria spp. and various fungal infections such as candidiasis. Of course, in this regard, proteins of the present invention may also be useful where a boost to the immune system generally may be desirable, i.e., in the treatment of cancer.

Autoimmune disorders which may be treated using a protein of the present invention include, for example, connective tissue disease, multiple sclerosis, systemic lupus erythematosus, rheumatoid arthritis, autoimmune pulmonary inflammation, Guillain-Barre syndrome, autoimmune thyroiditis, insulin dependent diabetes mellitis, myasthenia gravis, graft-versus-host disease and autoimmune inflammatory eye disease. Such a protein (or antagonists thereof, including antibodies) of the present invention may also to be useful in the treatment of allergic reactions and conditions (e.g., anaphylaxis, serum sickness, drug reactions, food allergies, insect venom allergies, mastocytosis, allergic rhinitis, hypersensitivity pneumonitis, urticaria, angioedema, eczema, atopic dermatitis, allergic contact dermatitis, erythema multiforme, Stevens-Johnson syndrome, allergic conjunctivitis, atopic keratoconjunctivitis, venereal keratoconjunctivitis, giant papillary conjunctivitis and contact allergies), such as asthma (particularly allergic asthma) or other respiratory problems. Other conditions, in which immune suppression is desired (including, for example, organ transplantation), may also be treatable using a protein (or antagonists thereof) of the present invention. The therapeutic effects of the polypeptides or antagonists thereof on allergic reactions can be evaluated by in vivo animals models such as the cumulative contact enhancement test (Lastborn et al., Toxicology 125: 59-66, 1998), skin prick test (Hoffmann et al., Allergy 54: 446-54,1999), guinea pig skin sensitization test (Vohr et al., Arch. Toxocol. 73: 501-9), and murine local lymph node assay (Kimber et al., J. Toxicol. Environ. Health 53: 563-79).

Using the proteins of the invention it may also be possible to modulate immune responses, in a number of ways. Down regulation may be in the form of inhibiting or blocking an immune response already in progress or may involve preventing the induction of an immune response. The functions of activated T cells may be inhibited by suppressing T cell responses or by inducing specific tolerance in T cells, or both. Immunosuppression of T cell responses is generally an active, non-antigen-specific, process which requires continuous exposure of the T cells to the suppressive agent. Tolerance, which involves inducing non-responsiveness or anergy in T cells, is distinguishable from immunosuppression in that it is generally antigen-specific and persists after exposure to the tolerizing agent has ceased. Operationally, tolerance can be demonstrated by the lack of a T cell response upon reexposure to specific antigen in the absence of the tolerizing agent.

Down regulating or preventing one or more antigen functions (including without limitation B lymphocyte antigen functions (such as, for example, B7)), e.g., preventing high level lymphokine synthesis by activated T cells, will be useful in situations of tissue, skin and organ transplantation and in graft-versus-host disease (GVHD). For example, blockage of T cell function should result in reduced tissue destruction in tissue transplantation. Typically, in tissue transplants, rejection of the transplant is initiated through its recognition as foreign by T cells, followed by an immune reaction that destroys the transplant. The administration of a therapeutic composition of the invention may prevent cytokine synthesis by immune cells, such as T cells, and thus acts as an immunosuppressant. Moreover, a lack of costimulation may also be sufficient to anergize the T cells, thereby inducing tolerance in a subject. Induction of long-term tolerance by B lymphocyte antigen-blocking reagents may avoid the necessity of repeated administration of these blocking reagents. To achieve sufficient immunosuppression or tolerance in a subject, it may also be necessary to block the function of a combination of B lymphocyte antigens.

The efficacy of particular therapeutic compositions in preventing organ transplant rejection or GVHD can be assessed using animal models that are predictive of efficacy in humans. Examples of appropriate systems which can be used include allogeneic cardiac grafts in rats and xenogeneic pancreatic islet cell grafts in mice, both of which have been used to examine the immunosuppressive effects of CTLA4Ig fusion proteins in vivo as described in Lenschow et al., Science 257:789-792 (1992) and Turka et al., Proc. Natl. Acad. Sci. USA, 89:11102-11105 (1992). In addition, murine models of GVHD (see Paul ed., Fundamental Immunology, Raven Press, New York, 1989, pp. 846-847) can be used to determine the effect of therapeutic compositions of the invention on the development of that disease.

Blocking antigen function may also be therapeutically useful for treating autoimmune diseases. Many autoimmune disorders are the result of inappropriate activation of T cells that are reactive against self-tissue and which promote the production of cytokines and autoantibodies involved in the pathology of the diseases. Preventing the activation of autoreactive T cells may reduce or eliminate disease symptoms. Administration of reagents which block stimulation of T cells can be used to inhibit T cell activation and prevent production of autoantibodies or T cell-derived cytokines which may be involved in the disease process. Additionally, blocking reagents may induce antigen-specific tolerance of autoreactive T cells which could lead to long-term relief from the disease. The efficacy of blocking reagents in preventing or alleviating autoimmune disorders can be determined using a number of well-characterized animal models of human autoimmune diseases. Examples include murine experimental autoimmune encephalitis, systemic lupus erythematosus in MRL/lpr/lpr mice or NZB hybrid mice, murine autoimmune collagen arthritis, diabetes mellitus in NOD mice and BB rats, and murine experimental myasthenia gravis (see Paul ed., Fundamental Immunology, Raven Press, New York, 1989, pp. 840-856).

Upregulation of an antigen function (e.g., a B lymphocyte antigen function), as a means of up regulating immune responses, may also be useful in therapy. Upregulation of immune responses may be in the form of enhancing an existing immune response or eliciting an initial immune response. For example, enhancing an immune response may be useful in cases of viral infection, including systemic viral diseases such as influenza, the common cold, and encephalitis.

Alternatively, anti-viral immune responses may be enhanced in an infected patient by removing T cells from the patient, costimulating the T cells in vitro with viral antigen-pulsed APCs either expressing a peptide of the present invention or together with a stimulatory form of a soluble peptide of the present invention and reintroducing the in vitro activated T cells into the patient. Another method of enhancing anti-viral immune responses would be to isolate infected cells from a patient, transfect them with a nucleic acid encoding a protein of the present invention as described herein such that the cells express all or a portion of the protein on their surface, and reintroduce the transfected cells into the patient. The infected cells would now be capable of delivering a costimulatory signal to, and thereby activate, T cells in vivo.

A KIRHy polypeptide may provide the necessary stimulation signal to T cells to induce a T cell mediated immune response against the transfected tumor cells. In addition, tumor cells which lack MHC class I or MHC class II molecules, or which fail to reexpress sufficient mounts of MHC class I or MHC class II molecules, can be transfected with nucleic acid encoding all or a portion of (e.g., a cytoplasmic-domain truncated portion) of an MHC class I alpha chain protein and β₂ microglobulin protein or an MHC class II alpha chain protein and an MHC class II beta chain protein to thereby express MHC class I or MHC class II proteins on the cell surface. Expression of the appropriate class I or class II MHC in conjunction with a peptide having the activity of a B lymphocyte antigen (e.g., B7-1, B7-2, B7-3) induces a T cell mediated immune response against the transfected tumor cell. Optionally, a gene encoding an antisense construct which blocks expression of an MHC class II associated protein, such as the invariant chain, can also be cotransfected with a DNA encoding a peptide having the activity of a B lymphocyte antigen to promote presentation of tumor associated antigens and induce tumor specific immunity. Thus, the induction of a T cell mediated immune response in a human subject may be sufficient to overcome tumor-specific tolerance in the subject.

The activity of a protein of the invention may, among other means, be measured by the following methods:

Suitable assays for thymocyte or splenocyte cytotoxicity include, without limitation, those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 3, In Vitro assays for Mouse Lymphocyte Function 3.1-3.19; Chapter 7, Immunologic studies in Humans); Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Takai et al., J. Immunol. 140:508-512, 1988; Bowman et al., J. Virology 61:1992-1998; Bertagnolli et al., Cellular Immunology 133:327-341, 1991; Brown et al., J. Immunol. 153:3079-3092, 1994.

Assays for T-cell-dependent immunoglobulin responses and isotype switching (which will identify, among others, proteins that modulate T-cell dependent antibody responses and that affect Th1/Th2 profiles) include, without limitation, those described in: Maliszewski, J. Immunol. 144:3028-3033, 1990; and Assays for B cell function: In vitro antibody production, Mond, J. J. and Brunswick, M. In Current Protocols in Immunology. J. E. e.a. Coligan eds. Vol 1 pp. 3.8.1-3.8.16, John Wiley and Sons, Toronto. 1994.

Mixed lymphocyte reaction (MLR) assays (which will identify, among others, proteins that generate predominantly Th1 and CTL responses) include, without limitation, those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Takai et al., J. Immunol. 140:508-512, 1988; Bertagnolli et al., J. Immunol. 149:3778-3783, 1992.

Dendritic cell-dependent assays (which will identify, among others, proteins expressed by dendritic cells that activate naive T-cells) include, without limitation, those described in: Guery et al., J. Immunol. 134:536-544, 1995; Inaba et al., J. Exp. Med. 173:549-559, 1991; Macatonia et al., J. Immunol. 154:5071-5079, 1995; Porgador et al., J. Exp. Med. 182:255-260, 1995; Nair et al., J. Virology 67:4062-4069, 1993; Huang et al., Science 264:961-965, 1994; Macatonia et al., J. Exp. Med. 169:1255-1264, 1989; Bhardwaj et al., J. Clin. Invest. 94:797-807, 1994; and Inaba et al., J. Exp. Med. 172:631-640, 1990.

Assays for lymphocyte survival/apoptosis (which will identify, among others, proteins that prevent apoptosis after superantigen induction and proteins that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Res. 53:1945-1951, 1993; Itoh et al., Cell 66:233-243, 1991; Zacharchuk, J. Immunol. 145:4037-4045, 1990; Zamai et al., Cytometry 14:891-897, 1993; Gorczyca et al., Int. J. Oncology 1:639-648, 1992.

Assays for proteins that influence early steps of T-cell commitment and development include, without limitation, those described in: Antica et al., Blood 84:111-117, 1994; Fine et al., Cellular Immunology 155:111-122, 1994; Galy et al., Blood 85:2770-2778, 1995; Toki et al., Proc. Nat. Acad. Sci. USA 88:7548-7551, 1991.

5.6.3 Anti-Inflammatory Activity of KIRHy

Compositions of the present invention may also exhibit anti-inflammatory activity. The anti-inflammatory activity may be achieved by providing a stimulus to cells involved in the inflammatory response, by inhibiting or promoting cell-cell interactions (such as, for example, cell adhesion), by inhibiting or promoting chemotaxis of cells involved in the inflammatory process, inhibiting or promoting cell extravasation, or by stimulating or suppressing production of other factors which more directly inhibit or promote an inflammatory response. Compositions with such activities can be used to treat inflammatory conditions including chronic or acute conditions), including without limitation intimation associated with infection (such as septic shock, sepsis or systemic inflammatory response syndrome (SIRS)), ischemia-reperfusion injury, endotoxin lethality, arthritis, complement-mediated hyperacute rejection, nephritis, cytokine or chemokine-induced lung injury, inflammatory bowel disease, Crohn's disease or resulting from over production of cytokines such as TNF or IL-1. Compositions of the invention may also be useful to treat anaphylaxis and hypersensitivity to an antigenic substance or material. Compositions of this invention may be utilized to prevent or treat conditions such as, but not limited to, sepsis, acute pancreatitis, endotoxin shock, cytokine induced shock, rheumatoid arthritis, chronic inflammatory arthritis, pancreatic cell damage from diabetes mellitus type 1, graft-vs-host disease, inflammatory bowel disease, inflammation associated with pulmonary disease, other autoimmune disease or inflammatory disease, an antiproliferative agent such as for acute or chronic myelogenous leukemia or in the prevention of premature labor secondary to intrauterine infections.

The immunosuppressive effects of the compositions of the invention against rheumatoid arthritis is determined in an experimental animal model system. The experimental model system is adjuvant induced arthritis in rats, and the protocol is described by J. Holoshitz, et al., 1983, Science, 219:56, or by B. Waksman et al., 1963, Int. Arch. Allergy Appl. Immunol., 23:129. Induction of the disease can be caused by a single injection, generally intradermally, of a suspension of killed Mycobacterium tuberculosis in complete Freund's adjuvant (CFA). The route of injection can vary, but rats may be injected at the base of the tail with an adjuvant mixture. The polypeptide is administered in phosphate buffered solution (PBS) at a dose of about 1-5 mg/kg. The control consists of administering PBS only.

The procedure for testing the effects of the test compound would consist of intradermally injecting killed Mycobacterium tuberculosis in CFA followed by immediately administering the test compound and subsequent treatment every other day until day 24. At 14, 15, 18, 20, 22, and 24 days after injection of Mycobacterium CFA, an overall arthritis score may be obtained as described by J. Holoskitz above. An analysis of the data would reveal that the test compound would have a dramatic affect on the swelling of the joints as measured by a decrease of the arthritis score.

5.7 Peptides

KIRHy peptides, such as fragments of the extracellular region, can be used to target toxins or radioisotopes to tumor cells in vivo by binding to or interacting with the cell surface antigens of the invention expressed on tumor or diseased cells. Much like an antibody, these fragments may specifically target cells expressing this antigen. Targeted delivery of these cytotoxic agents to the tumor cells would result in cell death and suppression of tumor growth. An example of the ability of an extracellular fragment binding to and activating its intact receptor by homophilic binding has been demonstrated with the CD84 receptor (Martin et al., J. Immunol. 167:3668-3676 (2001), herein incorporated by reference in its entirety).

Extracellular fragments of KIRHy can be used to modulate immune cells expressing the protein by KIRHy binding to and activating its own receptor on the cell surface resulting in stimulating the release of cytokines (such as interferon gamma from NK cells, T cells, B cells or myeloid cells, for example) that may enhance or suppress the immune system, depending on the type of cytokine released. Additionally, binding of these fragments to cells bearing KIRHy of the can activate these cells and stimulate proliferation. Some fragments may bind to the intact KIRHy and block activation signals and cytokine release by immune cells thereby having an immunosuppressive effect. Fragments that activate and stimulate the immune system may have anti-tumor properties by stimulating an immunological response resulting in immune-mediated tumor cell killing. The same fragments may result in stimulating the immune system to mount an enhanced response to foreign invaders such as viruses and bacteria. Fragments that suppress the immune response can be useful in treating lymphoproliferative disorders, auto-immune diseases, graft-vs-host disease, and inflammatory diseases, such as emphysema.

5.8 Other Binding Peptides or Small Molecules

Screening of organic compound or peptide libraries with recombinantly expressed KIRHy protein of the invention may be useful for identification of therapeutic molecules that function to specifically bind to or even inhibit the activity of KIRHy. Synthetic and naturally occurring products can be screened in a number of ways deemed routine to those of skill in the art. Random peptide libraries are displayed on phage (phage display) or on bacteria, such as on E. coli. These random peptide display libraries can be used to screen for peptides which interact with a known target which can be a protein or a polypeptide, such as a ligand or receptor, a biological or synthetic macromolecule, or organic or inorganic substances. By way of example, diversity libraries, such as random or combinatorial peptide or nonpeptide libraries can be screened for molecules that specifically bind to KIRHy polypeptides. Many libraries are known in the art that can be used, i.e. chemically synthesized libraries, recombinant (i.e. phage display libraries), and in vitro translation-based libraries. Techniques for creating and screening such random peptide display libraries are known in the art (Ladner et al., U.S. Pat. No. 5,223,409; Ladner et al., U.S. Pat. No. 4,946,778; Ladner et al., U.S. Pat. No. 5,403,484; Ladner et al., U.S. Pat. No. 5,571,698, all of which are herein incorporated by reference in their entirety) and random peptide display libraries and kits for screening such libraries are available commercially, for instance from Clontech (Palo Alto, Calif.), Invitrogen Inc. (San Diego, Calif.), New England Biolabs, Inc. (Beverly, Mass.), and Pharmacia KLB Biotechnology Inc. (Piscataway, N.J.). Random peptide display libraries can be screened using the KIRHy sequences disclosed herein to identify proteins which bind to KIRHy.

Examples of chemically synthesized libraries are described in Fodor et al., Science 251:767-773 (1991); Houghten et al., Nature 354:84-86 (1991); Lam et al., Nature 354:82-84 (1991); Medynski, Bio/Technology 12:709-710 (1994); Gallop et al., J. Med. Chem. 37:1233-1251 (1994); Ohlmeyer et al., Proc. Natl. Acad. Sci. USA 90:10922-10926 (1993); Erb et al., Proc. Natl. Acad. Sci. USA 91:11422-11426 (1994); Houghten et al., Biotechniques 13:412 (1992); Jayawickreme et al., Proc. Natl. Acad. Sci. USA 91:1614-1618 (1994); Salmon et al., Proc. Natl. Acad. Sci. USA 90:11708-11712 (1993); International Publication No. WO 93/20242; Brenner and Lerner, Proc. Natl. Acad. Sci. USA 89:5381-5383 (1992), all of which are herein incorporated by reference in their entirety.

Examples of phage display libraries are described in Scott and Smith, Science 249:386-390 (1990); Devlin et al., Science 249:404-406 (1990); Christian et al., J. Mol. Biol. 227:711-718 (1992); Lenstra, J. Immunol. Meth. 152:149-157 (1992); Kay et al., Gene 128:59-65 (1993); International Publication No. WO 94/18318, all of which are herein incorporated by reference in their entirety.

In vitro translation-based libraries include but are not limited to those described in International Publication No. WO 91/05058, and Mattheakis et al., Proc. Natl. Acad. Sci. USA 91:9022-9026 (1994), both of which are herein incorporated by reference in their entirety.

By way of examples of nonpeptide libraries, a benzodiazepine library (see for example, Bunin et al., Proc. Natl. Acad. Sci. USA 91:4708-4712 (1994), herein incorporated by reference in its entirety) can be adapted for use. Peptoid libraries (Simon et al., Proc. Natl. Acad. Sci. USA 89:9367-9371 (1992), herein incorporated by reference in its entirety) can also be used. Another example of a library that can be used, in which the amide functionalities in peptides have been permethylated to generate a chemically transformed combinatorial library, is described by Ostresh et al. (Proc. Natl. Acad. Sci. USA 91:11138-11142 (1994), herein incorporated by reference in its entirety).

Screening the libraries can be accomplished by any of a variety of commonly known methods. See, for example, the following references which disclose screening of peptide libraries: Parmley and Smith, Adv. Exp. Med. Biol. 251:215-218 (1989); Scott and Smith, Science 249:386-390 (1990); Fowlkes et al., Biotechniques 13:422-427 (1992); Oldenburg et al., Proc. Natl. Acad. Sci. USA 89:5393-5397 (1992); Yu et al., Cell 76:933-945 (1994); Staudt et al., Science 241:577-580 (1988); Bock et al., Nature 355:564-566 (1992); Tuerk et al., Proc. Natl. Acad. Sci. USA 89:6988-6992 (1992); Ellington et al., Nature 355:850-852 (1992); Rebar and Pabo, Science 263:671-673 (1993); and International Publication No. WO 94/18318, all of which are herein incorporated by reference in their entirety.

In a specific embodiment, screening can be carried out by contacting the library members with a KIRHy protein (or nucleic acid or derivative) immobilized on a solid phase and harvesting those library members that bind to the protein (or nucleic acid or derivative). Examples of such screening methods, termed “panning” techniques are described by way of example in Parmley and Smith, Gene 73:305-318 (1988); Fowlkes et al., Biotechniques 13:422-427 (1992); International Publication No. WO 94/18318, all of which are herein incorporated by reference in their entirety, and in references cited hereinabove.

In another embodiment, the two-hybrid system for selecting interacting protein in yeast (Fields and Song, Nature 340:245-246 (1989); Chien et al., Proc. Natl. Acad. Sci. USA 88:9578-9582 (1991), both of which are herein incorporated by reference in their entirety) can be used to identify molecules that specifically bind to KIRHy or a KIRHy derivative.

These “binding polypeptides” or small molecules which interact with KIRHy polypeptides can be used for tagging or targeting cells; for isolating homolog polypeptides by affinity purification; they can be directly or indirectly conjugated to drugs, toxins, radionuclides and the like. These binding polypeptides or small molecules can also be used in analytical methods such as for screening expression libraries and neutralizing activity, i.e., for blocking interaction between ligand and receptor, or viral binding to a receptor. The binding polypeptides or small molecules can also be used for diagnostic assays for determining circulating levels of KIRHy polypeptides of the invention; for detecting or quantitating soluble KIRHy polypeptides as marker of underlying pathology or disease. These binding polypeptides or small molecules can also act as KIRHy “antagonists” to block KIRHy binding and signal transduction in vitro and in vivo. These anti-KIRHy binding polypeptides or small molecules would be useful for inhibiting KIRHy activity or protein binding.

Binding polypeptides can also be directly or indirectly conjugated to drugs, toxins, radionuclides and the like, and these conjugates used for in vivo diagnostic or therapeutic applications. Binding peptides can also be fused to other polypeptides, for example an immunoglobulin constant chain or portions thereof, to enhance their half-life, and can be made multivalent (through, e.g. branched or repeating units) to increase binding affinity for KIRHy. For instance, binding polypeptides of the present invention can be used to identify or treat tissues or organs that express a corresponding anti-complementary molecule (receptor or antigen, respectively, for instance). More specifically, binding polypeptides or bioactive fragments or portions thereof, can be coupled to detectable or cytotoxic molecules and delivered to a mammal having cells, tissues or organs that express the anti-complementary molecule.

Suitable detectable molecules may be directly or indirectly attached to the binding polypeptide, and include radionuclides, enzymes, substrates, cofactors, inhibitors, fluorescent markers, chemiluminescent markers, magnetic particles and the like. Suitable cytotoxic molecules may be directly or indirectly attached to the binding polypeptide, and include bacterial or plant toxins (for instance, diphtheria toxin, Pseudomonas exotoxin, ricin, abrin and the like), as well as therapeutic radionuclides, such as iodine-131, rhenium-188, or yttrium-90 (either directly attached to the binding polypeptide, or indirectly attached through a means of a chelating moiety, for instance). Binding polypeptides may also be conjugated to cytotoxic drugs, such as adriamycin. For indirect attachment of a detectable or cytotoxic molecule, the detectable or cytotoxic molecule can be conjugated with a member of a complementary/anticomplementary pair, where the other member is bound to the binding polypeptide. For these purposes, biotin/streptavidin is an exemplary complementary/anticomplementary pair.

In another embodiment, binding polypeptide-toxin fusion proteins can be used for targeted cell or tissue inhibition or ablation (for instance, to treat cancer cells or tissues). Alternatively, if the binding polypeptide has multiple functional domains (i.e., an activation domain or a ligand binding domain, plus a targeting domain), a fusion protein including only the targeting domain may be suitable for directing a detectable molecule, a cytotoxic molecule, or a complementary molecule to a cell or tissue type of interest. In instances where the domain only fusion protein includes a complementary molecule, the anti-complementary molecule can be conjugated to a detectable or cytotoxic molecule. Such domain-complementary molecule fusion proteins thus represent a generic targeting vehicle for cell/tissue-specific delivery of generic anti-complementary-detectable/cytotoxic molecule conjugates.

5.9 Diseases Amenable to Anti-KIRHy Targeting

In one aspect, the present invention provides reagents and methods useful for treating diseases and conditions wherein cells associated with the disease or disorder express KIRHy such as, but not limited to, AML. In addition, these diseases can include cancers, and other hyperproliferative conditions, such as hyperplasia, psoriasis, contact dermatitis, immunological disorders, wound healing, arthritis, and autoimmune disease. Whether the cells associated with a disease or condition express KIRHy can be determined using the diagnostic methods described herein.

Comparisons of KIRHy mRNA and protein expression levels between diseased cells, tissue or fluid (blood, lymphatic fluid, etc.) and corresponding normal samples are made to determine if the patient will be responsive to therapy targeting KIRHy antigens of the invention. Methods for detecting and quantifying the expression of KIRHy mRNA or protein use standard nucleic acid and protein detection and quantitation techniques that are well known in the art and are described in Sambrook, et al., Molecular Cloning: A Laboratory Manual, Cold Spring Harbor Laboratory, NY (1989) or Ausubel, et al., Current Protocols in Molecular Biology, John Wiley & Sons, New York, N.Y. (1989), both of which are incorporated herein by reference in their entirety. Standard methods for the detection and quantification of KIRHy mRNA include in situ hybridization using labeled KIRHy riboprobes (Gemou-Engesaeth, et al., Pediatrics 109: E24-E32 (2002), herein incorporated by reference in its entirety), Northern blot and related techniques using KIRHy polynucleotide probes (Kunzli, et al., Cancer 94: 228 (2002), herein incorporated by reference in its entirety), RT-PCR analysis using KIRHy-specific primers (Angchaiskisiri, et al., Blood 99:130 (2002), herein incorporated by reference in its entirety), and other amplification detection methods, such as branched chain DNA solution hybridization assay (Jardi, et al., J. Viral Hepat. 8:465-471 (2001), herein incorporated by reference in its entirety), transcription-mediated amplification (Kimura, et al., J. Clin. Microbiol. 40:439-445 (2002), herein incorporated by reference in its entirety), microarray products, such as oligos, cDNAs, and monoclonal antibodies, and real-time PCR (Simpson, et al., Molec. Vision, 6:178-183 (2000), herein incorporated by reference in its entirety). Standard methods for the detection and quantification of KIRHy protein include western blot analysis (Sambrook, 1989 supra, Ausubel, 1989 supra)), immunocytochemistry (Racila, et al., Proc. Natl. Acad. Sci. USA 95:4589-4594 (1998), herein incorporated by reference in its entirety), and a variety of immunoassays, including enzyme-linked immunosorbant assay (ELISA), radioimmuno assay (RIA), and specific enzyme immunoassay (EIA) (Sambrook, 1989 supra, Ausubel, 1989 supra). Peripheral blood cells can also be analyzed for KIRHy expression using flow cytometry using, for example, immunomagnetic beads specific for KIRHy (Racila, 1998 supra) or biotinylated KIRHy antibodies (Soltys, et al., J. Immunol. 168:1903 (2002), herein incorporated by reference in its entirety).

Yet another related aspect of the invention is directed to methods for gauging tumor aggressiveness by determining the levels of KIRHy protein or mRNA in tumor cells compared to the corresponding normal cells (Orlandi, et al., Cancer Res. 62:567 (2002), herein incorporated by reference in its entirety). In one embodiment, the disease or disorder is a cancer.

The diseases treatable by methods of the present invention preferably occur in mammals. Mammals include, for example, humans and other primates, as well as pet or companion animals such as dogs and cats, laboratory animals such as rats, mice and rabbits, and farm animals such as horses, pigs, sheep, and cattle.

Tumors or neoplasms include growths of tissue cells in which the multiplication of the cells is uncontrolled and progressive. Some such growths are benign, but others are termed “malignant” and may lead to death of the organism. Malignant neoplasms or “cancers” are distinguished from benign growths in that, in addition to exhibiting aggressive cellular proliferation, they may invade surrounding tissues and metastasize. Moreover, malignant neoplasms are characterized in that they show a greater loss of differentiation (greater “dedifferentiation”), and greater loss of their organization relative to one another and their surrounding tissues. This property is also called “anaplasia.”

Neoplasms treatable by the present invention also include solid phase tumors/malignancies, i.e., carcinomas, locally advanced tumors and human soft tissue sarcomas. Carcinomas include those malignant neoplasms derived from epithelial cells that infiltrate (invade) the surrounding tissues and give rise to metastatic cancers, including lymphatic metastases. Adenocarcinomas are carcinomas derived from glandular tissue, or which form recognizable glandular structures. Another broad category or cancers includes sarcomas, which are tumors whose cells are embedded in a fibrillar or homogeneous substance like embryonic connective tissue. The invention also enables treatment of cancers of the myeloid or lymphoid systems, including leukemias, lymphomas and other cancers that typically do not present as a tumor mass, but are distributed in the vascular or lymphoreticular systems. The invention also enables treatment of non-malignant and/or pre-cancerous proliferative disorders such as hypereosinophilic syndrome and other myelodysplastic/myeloproliferative disorders.

The types of cancer or tumor cells that may be amenable to treatment according to the invention include AML. Other types of cancer that may benefit from KIRHy targeting include: chronic lymphocytic leukemia, chronic myelomonocytic leukemia, chronic myelocytic leukemia, cutaneous T-cell lymphoma, hairy cell leukemia, erythroleukemia, chronic myeloid (granulocytic) leukemia, Hodgkin's disease, and non-Hodgkin's lymphoma, gastrointestinal cancers including esophageal cancer, stomach cancer, colon cancer, colorectal cancer, polyps associated with colorectal neoplasms, pancreatic cancer and gallbladder cancer, cancer of the adrenal cortex, ACTH-producing tumor, bladder cancer, brain cancer including intrinsic brain tumors, neuroblastomas, astrocytic brain tumors, gliomas, and metastatic tumor cell invasion of the central nervous system, Ewing's sarcoma, head and neck cancer including mouth cancer and larynx cancer, kidney cancer including renal cell carcinoma, liver cancer, lung cancer including small and non-small cell lung cancers, malignant peritoneal effusion, malignant pleural effusion, skin cancers including malignant melanoma, tumor progression of human skin keratinocytes, squamous cell carcinoma, basal cell carcinoma, and hemangiopericytoma, mesothelioma, Kaposi's sarcoma, bone cancer including osteomas and sarcomas such as fibrosarcoma and osteosarcoma, cancers of the female reproductive tract including uterine cancer, endometrial cancer, ovarian cancer, ovarian (germ cell) cancer and solid tumors in the ovarian follicle, vaginal cancer, cancer of the vulva, and cervical cancer; breast cancer (small cell and ductal), penile cancer, prostate cancer, retinoblastoma, testicular cancer, thyroid cancer, trophoblastic neoplasms, and Wilms' tumor.

The invention is particularly illustrated herein in reference to treatment of certain types of experimentally defined cancers. In these illustrative treatments, standard state-of-the-art in vitro and in vivo models have been used. These methods can be used to identify agents that can be expected to be efficacious in in vivo treatment regimens. However, it will be understood that the method of the invention is not limited to the treatment of these tumor types, but extends to any cancer derived from any organ system. As demonstrated in the Examples, KIRHy is highly expressed in hematopoietic cell-related disorders such as AML and histiocytic lymphoma. Leukemias can result from uncontrolled B cell proliferation initially within the bone marrow before disseminating to the peripheral blood, spleen, lymph nodes and finally to other tissues. Uncontrolled B cell proliferation also may result in the development of lymphomas that arise within the lymph nodes and then spread to the blood and bone marrow. Targeting KIRHy is useful in treating myeloproliferative disorders, B cell malignancies, leukemias, lymphomas and myelomas including but not limited to multiple myeloma, Burkitt's lymphoma, cutaneous B cell lymphoma, primary follicular cutaneous B cell lymphoma, B lineage acute lymphoblastic leukemia (ALL), B cell non-Hodgkin's lymphoma (NHL), B cell chronic lymphocytic leukemia (CLL), acute lymphoblastic leukemia, hairy cell leukemia (HCL), acute myelogenous leukemia, acute myelomonocytic leukemia, chronic myelogenous leukemia, lymphosarcoma cell leukemia, splenic marginal zone lymphoma, diffuse large B cell lymphoma, B cell large cell lymphoma, malignant lymphoma, prolymphocytic leukemia (PLL), lymphoplasma cytoid lymphoma, mantle cell lymphoma, mucosa-associated lymphoid tissue (MALT) lymphoma, primary thyroid lymphoma, intravascular malignant lymphomatosis, splenic lymphoma, Hodgkin's Disease, and intragraft angiotropic large-cell lymphoma. Expression of KIRHy has also been demonstrated in Example 4 to be expressed in acute monocytic leukemia, acute myeloid leukemia, acute myelogenous leukemia, anaplastic large T cell lymphoma, B cell lymphoma, chronic myelogenous leukemia, diffuse large B cell lymphoma, follicular lymphoma, histiocytic lymphoma, Hodgkin's lymphoma, large B cell lymphoma, myeloma, non-Hodgkin's lymphoma, and plasmacytoma cell lines and tissue, and may be treated with KIRHy antibodies. Other diseases that may be treated by the methods of the present invention include myelodysplastic syndromes, hypereosinophilic syndrome, multicentric Castleman's disease, primary amyloidosis, Franklin's disease, Seligmann's disease, primary effusion lymphoma, post-transplant lymphoproliferative disease (PTLD) [associated with EBV infection], paraneoplastic pemphigus, chronic lymphoproliferative disorders, X-linked lymphoproliferative syndrome (XLP), acquired angioedema, angioimmunoblastic lymphadenopathy with dysproteinemia, Herman's syndrome, post-splenectomy syndrome, congenital dyserythropoietic anemia type III, lymphoma-associated hemophagocytic syndrome (LAHS), necrotizing ulcerative stomatitis, Kikuchi's disease, lymphomatoid granulomatosis, Richter's syndrome, polycythemic vera (PV), Gaucher's disease, Gougerot-Sjogren syndrome, Kaposi's sarcoma, cerebral lymphoplasmocytic proliferation (Bind and Neel syndrome), X-linked lymphoproliferative disorders, pathogen associated disorders such as mononucleosis (Epstein Barr Virus), lymphoplasma cellular disorders, post-transplantational plasma cell dyscrasias, and Good's syndrome.

Therapeutic compositions of the invention may be effective in adult and pediatric oncology including in solid phase tumors/malignancies, locally advanced tumors, human soft tissue sarcomas, metastatic cancer, including lymphatic metastases, blood cell malignancies, including multiple myeloma, acute and chronic leukemias and lymphomas, head and neck cancers, including mouth cancer, larynx cancer, and thyroid cancer, lung cancers including small cell carcinoma and non-small cell cancers, breast cancers including small cell carcinoma and ductal carcinoma, gastrointestinal cancers including esophageal cancer, stomach cancer, colon cancer, colorectal cancer and polyps associated with colorectal neoplasia, pancreatic cancers, liver cancer, urologic cancers including bladder cancer and prostate cancer, malignancies of the female genital tract including ovarian carcinoma, uterine (including endometrial) cancers, and solid tumor in the ovarian follicle, kidney cancers including renal cell carcinoma, brain cancers including intrinsic brain tumors, neuroblastoma, astrocytic brain tumors, gliomas, metastatic tumor cell invasion in the central nervous system, bone cancers including osteomas, sarcomas including fibrosarcoma and osteosarcoma, skin cancers including malignant melanoma, tumor progression of human skin keratinocytes, squamous cell carcinoma, basal cell carcinoma, hemangiopericytoma, and Karposi's sarcoma.

Autoimmune diseases can be associated with hyperactive B cell activity that results in autoantibody production. Additionally, autoimmune diseases can be associated with uncontrolled protease activity (Wernike et al., Arthritis Rheum. 46:64-74 (2002)) and aberrant cytokine activity (Rodenburg et al., Ann. Rheum. Dis. 58:648-652 (1999), both of which are herein incorporated by reference in their entirety). Inhibition of the development of autoantibody-producing cells or proliferation of such cells may be therapeutically effective in decreasing the levels of autoantibodies in autoimmune diseases. Inhibition of protease activity may reduce the extent of tissue invasion and inflammation associated with autoimmune diseases including but not limited to systemic lupus erythematosus, Hashimoto thyroiditis, Sjogren's syndrome, pericarditis lupus, Crohn's Disease, graft-verses-host disease, Graves' disease, myasthenia gravis, autoimmune hemolytic anemia, autoimmune thrombocytopenia, asthma, cryoglubulinemia, primary biliary sclerosis, pernicious anemia, Waldenstrom macroglobulinemia, hyperviscosity syndrome, macroglobulinemia, cold agglutinin disease, monoclonal gammopathy of undetermined origin, anetoderma and POEMS syndrome (polyneuropathy, organomegaly, endocrinopathy, M component, skin changes), connective tissue disease, multiple sclerosis, cystic fibrosis, rheumatoid arthritis, autoimmune pulmonary inflammation, psoriasis, Guillain-Barre syndrome, autoimmune thyroiditis, insulin dependent diabetes mellitis, autoimmune inflammatory eye disease, Goodpasture's disease, Rasmussen's encephalitis, dermatitis herpetiformis, thyoma, autoimmune polyglandular syndrome type 1, primary and secondary membranous nephropathy, cancer-associated retinopathy, autoimmune hepatitis type 1, mixed cryoglobulinemia with renal involvement, cystoid macular edema, endometriosis, IgM polyneuropathy (including Hyper IgM syndrome), demyelinating diseases, angiomatosis, and monoclonal gammopathy.

Targeting KIRHy may also be useful in the treatment of allergic reactions and conditions e.g., anaphylaxis, serum sickness, drug reactions, food allergies, insect venom allergies, mastocytosis, allergic rhinitis, hypersensitivity pneumonitis, urticaria, angioedema, eczema, atopic dermatitis, allergic contact dermatitis, erythema multiforme, Stevens-Johnson syndrome, allergic conjunctivitis, atopic keratoconjunctivitis, venereal keratoconjunctivitis, giant papillary conjunctivitis, allergic gastroenteropathy, inflammatory bowel disorder (IBD), and contact allergies, such as asthma (particularly allergic asthma), or other respiratory problems.

Targeting KIRHy may also be useful in the management or prevention of transplant rejection in patients in need of transplants such as stem cells, tissue or organ transplant. Thus, one aspect of the invention may find therapeutic utility in various diseases (such as those usually treated with transplantation, including without limitation, aplastic anemia and paroxysmal nocturnal hemoglobinuria) as wells in repopulating the stem cell compartment post irradiation/chemotherapy, either in vivo or ex vivo (i.e. in conjunction with bone marrow transplantation or with peripheral progenitor cell transplantation (homologous or heterologous) as normal cells or genetically manipulated for gene therapy.

Targeting of KIRHy may also be possible to modulate immune responses, in a number of ways. Down regulation may be in the form of inhibiting or blocking an immune response already in progress or may involve preventing the induction of an immune response. Down regulating or preventing one or more antigen functions (including without limitation B lymphocyte antigen functions, e.g., modulating or preventing high level lymphokine synthesis by activated T cells, will be useful in situations of tissue, skin and organ transplantation and in graft-versus-host disease (GVHD). For example, blockage of T cell function should result in reduced tissue destruction in tissue transplantation. Typically, in tissue transplants, rejection of the transplant is initiated through its recognition as foreign by T cells, followed by an immune reaction that destroys the transplant. The administration of a therapeutic composition of the invention may prevent cytokine synthesis by immune cells, such as T cells, and thus acts as an immunosuppressant. Moreover, a lack of costimulation may also be sufficient to anergize the T cells, thereby inducing tolerance in a subject. Induction of long-term tolerance by B lymphocyte antigen-blocking reagents may avoid the necessity of repeated administration of these blocking reagents. To achieve sufficient immunosuppression or tolerance in a subject, it may also be necessary to block the function of a combination of B lymphocyte antigens.

The efficacy of particular therapeutic compositions in preventing organ transplant rejection or GVHD can be assessed using animal models that are predictive of efficacy in humans. Examples of appropriate systems which can be used include allogeneic cardiac grafts in rats and xenogeneic pancreatic islet cell grafts in mice, both of which have been used to examine the immunosuppressive effects of CTLA4Ig fusion proteins in vivo as described in Lenschow et al., Science 257:789-792 (1992) and Turka et al., Proc. Natl. Acad. Sci. USA, 89:11102-11105 (1992), both of which are herein incorporated by reference. In addition, murine models of GVHD (see Paul ed., Fundamental Immunology, Raven Press, New York, 1989, pp. 846-847, herein incorporated by reference) can be used to determine the effect of therapeutic compositions of the invention on the development of that disease.

5.10 Administration

The anti-KIRHy monoclonal antibodies used in the practice of a method of the invention may be formulated into pharmaceutical compositions comprising a carrier suitable for the desired delivery method. Suitable carriers include any material which when combined with the anti-KIRHy antibodies retains the anti-tumor function of the antibody and is nonreactive with the subject's immune systems. Examples include, but are not limited to, any of a number of standard pharmaceutical carriers such as sterile phosphate buffered saline solutions, bacteriostatic water, and the like.

The anti-KIRHy antibody formulations may be administered via any route capable of delivering the antibodies to the tumor site. Potentially effective routes of administration include, but are not limited to, intravenous, intraperitoneal, intramuscular, intratumor, intradermal, and the like. The preferred route of administration is by intravenous injection. A preferred formulation for intravenous injection comprises anti-KIRHy mAbs in a solution of preserved bacteriostatic water, sterile unpreserved water, and/or diluted in polyvinylchloride or polyethylene bags containing 0.9% sterile sodium chloride for Injection, USP. The anti-KIRHy mAb preparation may be lyophilized and stored as a sterile powder, preferably under vacuum, and then reconstituted in bacteriostatic water containing, for example, benzyl alcohol preservative, or in sterile water prior to injection.

Treatment will generally involve the repeated administration of the anti-KIRHy antibody preparation via an acceptable route of administration such as intravenous injection (IV), typically at a dose in the range of about 0.1 to about 10 mg/kg body weight; however other exemplary doses in the range of 0.01 mg/kg to about 100 mg/kg are also contemplated. Doses in the range of 10-500 mg mAb per week may be effective and well tolerated. Rituximab (RITUXAN®), a chimeric CD20 antibody used to treat B-cell lymphoma, non-Hodgkin's lymphoma, and relapsed indolent lymphoma, is typically administered at 375 mg/m² by IV infusion once a week for 4 to 8 doses. Sometimes a second course is necessary, but no more than 2 courses are allowed. An effective dosage range for RITUXAN® would be 50 to 500 mg/m² (Maloney, et al., Blood 84: 2457-2466 (1994); Davis, et al., J. Clin. Oncol. 18: 3135-3143 (2000), both of which are herein incorporated by reference in their entirety). Based on clinical experience with Trastuzumab (HERCEPTIN®), a humanized monoclonal antibody used to treat HER2 (human epidermal growth factor 2)-positive metastatic breast cancer (Slamon, et al., Mol Cell Biol. 9: 1165 (1989), herein incorporated by reference in its entirety), an initial loading dose of approximately 4 mg/kg patient body weight IV followed by weekly doses of about 2 mg/kg IV of the anti-KIRHy mAb preparation may represent an acceptable dosing regimen (Slamon, et al., N. Engl. J. Med. 344: 783(2001), herein incorporated by reference in its entirety). Preferably, the initial loading dose is administered as a 90 minute or longer infusion. The periodic maintenance dose may be administered as a 30 minute or longer infusion, provided the initial dose was well tolerated. However, as one of skill in the art will understand, various factors will influence the ideal dose regimen in a particular case. Such factors may include, for example, the binding affinity and half life of the mAb or mAbs used, the degree of KIRHy overexpression in the patient, the extent of circulating shed KIRHy antigen, the desired steady-state antibody concentration level, frequency of treatment, and the influence of chemotherapeutic agents used in combination with the treatment method of the invention.

Treatment can also involve anti-KIRHy antibodies conjugated to radioisotopes. Studies using radiolabeled-anticarcinoembryonic antigen (anti-CEA) monoclonal antibodies, provide a dosage guideline for tumor regression of 2-3 infusions of 30-80 mCi/m² (Behr, et al. Clin, Cancer Res. 5(10 Suppl.): 3232s-3242s (1999), Juweid, et al., J. Nucl. Med. 39:34-42 (1998), both of which are herein incorporated in their entirety).

Alternatively, dendritic cells transfected with mRNA encoding KIRHy can be used as a vaccine to stimulate T-cell mediated anti-tumor responses. Studies with dendritic cells transfected with prostate-specific antigen mRNA suggest a 3 cycles of intravenous administration of 1×10⁷-5×10⁷ cells for 2-6 weeks concomitant with an intradermal injection of 10⁷ cells may provide a suitable dosage regimen (Heiser, et al., J. Clin. Invest. 109:409-417 (2002); Hadzantonis and O'Neill, Cancer Biother. Radiopharm. 1:11-22 (1999), both of which are herein incorporated in their entirety). Other exemplary doses of between 1×10⁵ to 1×10⁹ or 1×10⁶ to 1×10⁸ cells are also contemplated.

Naked DNA vaccines using plasmids encoding KIRHy can induce an immunologic anti-tumor response. Administration of naked DNA by direct injection into the skin and muscle is not associated with limitations encountered using viral vectors, such as the development of adverse immune reactions and risk of insertional mutagenesis (Hengge, et al., J. Invest. Dermatol. 116:979 (2001), herein incorporated in its entirety). Studies have shown that direct injection of exogenous cDNA into muscle tissue results in a strong immune response and protective immunity (Ilan, Curr. Opin. Mol. Ther. 1:116-120 (1999), herein incorporated in its entirety). Physical (gene gun, electroporation) and chemical (cationic lipid or polymer) approaches have been developed to enhance efficiency and target cell specificity of gene transfer by plasmid DNA (Nishikawa and Huang, Hum. Gene Ther. 12:861-870 (2001), herein incorporated in its entirety). Plasmid DNA can also be administered to the lungs by aerosol delivery (Densmore, et al., Mol. Ther. 1:180-188 (2000)). Gene therapy by direct injection of naked or lipid-coated plasmid DNA is envisioned for the prevention, treatment, and cure of diseases such as cancer, acquired immunodeficiency syndrome, cystic fibrosis, cerebrovascular disease, and hypertension (Prazeres, et al., Trends Biotechnol. 17:169-174 (1999); Weihl, et al., Neurosurgery 44:239-252 (1999), both of which are herein incorporated in their entirety). HIV-1 DNA vaccine dose-escalating studies indicate administration of 30-300 μg/dose as a suitable therapy (Weber, et al., Eur. J. Clin. Microbiol. Infect. Dis. 20: 800 (2001), herein incorporated in its entirety. Naked DNA injected intracerebrally into the mouse brain was shown to provide expression of a reporter protein, wherein expression was dose-dependent and maximal for 150 μg DNA injected (Schwartz, et al., Gene Ther. 3:405-411 (1996), herein incorporated in its entirety). Gene expression in mice after intramuscular injection of nanospheres containing 1 microgram of beta-galactosidase plasmid was greater and more prolonged than was observed after an injection with an equal amount of naked DNA or DNA complexed with Lipofectamine (Truong, et al., Hum. Gene Ther. 9:1709-1717 (1998), herein incorporated in its entirety). In a study of plasmid-mediated gene transfer into skeletal muscle as a means of providing a therapeutic source of insulin, wherein four plasmid constructs comprising a mouse furin cDNA transgene and rat proinsulin cDNA were injected into the calf muscles of male Balb/c mice, the optimal dose for most constructs was 100 micrograms plasmid DNA (Kon, et al. J. Gene Med. 1:186-194 (1999), herein incorporated in its entirety). Other exemplary doses of 1-1000 μg/dose or 10-500 μg/dose are also contemplated.

Optimally, patients should be evaluated for the level of circulating shed KIRHy antigen in serum in order to assist in the determination of the most effective dosing regimen and related factors. Such evaluations may also be used for monitoring purposes throughout therapy, and may be useful to gauge therapeutic success in combination with evaluating other parameters.

5.10.1 KIRHy Targeting Compositions

Compositions for targeting KIRHy-expressing cells are within the scope of the present invention. Pharmaceutical compositions comprising antibodies are described in detail in, for example, U.S. Pat. No. 6,171,586, herein incorporated in its entirety. Such compositions comprise a therapeutically or prophylactically effective amount an antibody, or a fragment, variant, derivative or fusion thereof as described herein, in admixture with a pharmaceutically acceptable agent. Typically, the KIRHy targeting agent will be sufficiently purified for administration to an animal.

The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents [such as ethylenediamine tetraacetic acid (EDTA)]; complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants. (Remington's Pharmaceutical Sciences, 18th Edition, Ed. A. R. Gennaro, Mack Publishing Company, (1990), herein incorporated in its entirety).

The optimal pharmaceutical composition will be determined by one skilled in the art depending upon, for example, the intended route of administration, delivery format, and desired dosage. See, for example, Remington's Pharmaceutical Sciences, supra. Such compositions may influence the physical state, stability, rate of in vivo release, and rate of in vivo clearance of the KIRHy targeting agent.

The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute therefor. In one embodiment of the present invention, KIRHy targeting agent compositions may be prepared for storage by mixing the selected composition having the desired degree of purity with optional formulation agents (Remington's Pharmaceutical Sciences, supra) in the form of a lyophilized cake or an aqueous solution. Further, the binding agent product may be formulated as a lyophilizate using appropriate excipients such as sucrose.

The pharmaceutical compositions can be selected for parenteral delivery. Alternatively, the compositions may be selected for inhalation or for delivery through the digestive tract, such as orally. The preparation of such pharmaceutically acceptable compositions is within the skill of the art. The formulation components are present in concentrations that are acceptable to the site of administration. For example, buffers are used to maintain the composition at physiological pH or at slightly lower pH, typically within a pH range of from about 5 to about 8. When parenteral administration is contemplated, the therapeutic compositions for use in this invention may be in the form of a pyrogen-free, parenterally acceptable aqueous solution comprising the KIRHy targeting agent in a pharmaceutically acceptable vehicle. A particularly suitable vehicle for parenteral injection is sterile distilled water in which a KIRHy targeting agent is formulated as a sterile, isotonic solution, properly preserved. Yet another preparation can involve the formulation of the desired molecule with an agent, such as injectable microspheres, bio-erodible particles, polymeric compounds (polylactic acid, polyglycolic acid), beads, or liposomes, that provides for the controlled or sustained release of the product which may then be delivered via a depot injection. Hyaluronic acid may also be used, and this may have the effect of promoting sustained duration in the circulation. Other suitable means for the introduction of the desired molecule include implantable drug delivery devices.

In another aspect, pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances that increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also contain suitable stabilizers or agents to increase the solubility of the compounds and allow for the preparation of highly concentrated solutions.

In another embodiment, a pharmaceutical composition may be formulated for inhalation. For example, a KIRHy targeting agent may be formulated as a dry powder for inhalation. Polypeptide or nucleic acid molecule inhalation solutions may also be formulated with a propellant for aerosol delivery. In yet another embodiment, solutions may be nebulized. Pulmonary administration is further described in PCT Application No. PCT/US94/001875, herein incorporated in its entirety, which describes pulmonary delivery of chemically modified proteins.

It is also contemplated that certain formulations may be administered orally. In one embodiment of the present invention, KIRHy targeting agents that are administered in this fashion can be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents can be included to facilitate absorption of the binding agent molecule. Diluents, flavorings, low melting point waxes, vegetable oils, lubricants, suspending agents, tablet disintegrating agents, and binders may also be employed.

Pharmaceutical compositions for oral administration can also be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combining active compounds with solid excipient and processing the resultant mixture of granules (optionally, after grinding) to obtain tablets or dragee cores. Suitable auxiliaries can be added, if desired. Suitable excipients include carbohydrate or protein fillers, such as sugars, including lactose, sucrose, mannitol, and sorbitol; starch from corn, wheat, rice, potato, or other plants; cellulose, such as methyl cellulose, hydroxypropylmethyl-cellulose, or sodium carboxymethylcellulose; gums, including arabic and tragacanth; and proteins, such as gelatin and collagen. If desired, disintegrating or solubilizing agents may be added, such as the cross-linked polyvinyl pyrrolidone, agar, and alginic acid or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations that can be used orally also include push-fit capsules made of gelatin, as well as soft, sealed capsules made of gelatin and a coating, such as glycerol or sorbitol. Push-fit capsules can contain active ingredients mixed with fillers or binders, such as lactose or starches, lubricants, such as talc or magnesium stearate, and, optionally, stabilizers. In soft capsules, the KIRHy targeting agent may be dissolved or suspended in suitable liquids, such as fatty oils, liquid, or liquid polyethylene glycol with or without stabilizers.

Another pharmaceutical composition may involve an effective quantity of KIRHy targeting agent in a mixture with non-toxic excipients that are suitable for the manufacture of tablets. By dissolving the tablets in sterile water, or other appropriate vehicle, solutions can be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving KIRHy targeting agents in sustained- or controlled-delivery formulations. Techniques for formulating a variety of other sustained- or controlled-delivery means, such as liposome carriers, bio-erodible microparticles or porous beads and depot injections, are also known to those skilled in the art. See, for example, PCT/US93/00829, herein incorporated in its entirety, that describes controlled release of porous polymeric microparticles for the delivery of pharmaceutical compositions. Additional examples of sustained-release preparations include semipermeable polymer matrices in the form of shaped articles, e.g. films, or microcapsules. Sustained release matrices may include polyesters, hydrogels, polylactides (U.S. Pat. No. 3,773,919; European Patent No. EP 58,481), copolymers of L-glutamic acid and gamma ethyl-L-glutamate (Sidman et al., Biopolymers, 22:547-556 (1983)), poly (2-hydroxyethyl-methacrylate) (Langer et al., J Biomed Mater Res, 15:167-277, (1981)) and (Langer et al., Chem Tech, 12:98-105(1982)), ethylene vinyl acetate (Langer et al., supra) or poly-D (−)-3-hydroxybutyric acid (European Patent No. EP 133,988, all of which are herein incorporated in their entirety). Sustained-release compositions also include liposomes, which can be prepared by any of several methods known in the art. See e.g., Epstein, et al., Proc Natl Acad Sci (USA), 82:3688-3692 (1985); European Patent Nos. EP 36,676, EP 88,046, and EP 143,949, all of which are herein incorporated by reference in their entirety.

The pharmaceutical composition to be used for in vivo administration typically must be sterile. This may be accomplished by filtration through sterile filtration membranes. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or a dehydrated or lyophilized powder. Such formulations may be stored either in a ready-to-use form or in a form (e.g., lyophilized) requiring reconstitution prior to administration.

In a specific embodiment, the present invention is directed to kits for producing a single-dose administration unit. The kits may each contain both a first container having a dried KIRHy targeting agent and a second container having an aqueous formulation. Also included within the scope of this invention are kits containing single and multi-chambered pre-filled syringes (e.g., liquid syringes and lyosyringes).

5.10.2 Dosage

An effective amount of a pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which KIRHy targeting agent is being used, the route of administration, and the size (body weight, body surface or organ size) and condition (the age and general health) of the patient. Accordingly, the clinician may titer the dosage and modify the route of administration to obtain the optimal therapeutic effect. A typical dosage may range from about 0.1 mg/kg to up to about 100 mg/kg or more, depending on the factors mentioned above. In other embodiments, the dosage may range from 0.1 mg/kg up to about 100 mg/kg; or 0.01 mg/kg to 1 g/kg; or 1 mg/kg up to about 100 mg/kg or 5 mg/kg up to about 100 mg/kg. In other embodiments, the dosage may range from 10 mCi to 100 mCi per dose for radioimmunotherapy, from about 1×10⁷-5×10⁷ cells or 1×10⁵ to 1×10⁹ cells or 1×10⁶ to 1×10⁸ cells per injection or infusion, or from 30 μg to 300 μg naked DNA per dose or 1-1000 μg/dose or 10-500 μg/dose, depending on the factors listed above.

For any compound, the therapeutically effective dose can be estimated initially either in cell culture assays or in animal models such as mice, rats, rabbits, dogs, or pigs. An animal model may also be used to determine the appropriate concentration range and route of administration. Such information can then be used to determine useful doses and routes for administration in humans.

The exact dosage will be determined in light of factors related to the subject requiring treatment. Dosage and administration are adjusted to provide sufficient levels of the active compound or to maintain the desired effect. Factors that may be taken into account include the severity of the disease state, the general health of the subject, the age, weight, and gender of the subject, time and frequency of administration, drug combination(s), reaction sensitivities, and response to therapy. Long-acting pharmaceutical compositions may be administered every 3 to 4 days, every week, or biweekly depending on the half-life and clearance rate of the particular formulation.

The frequency of dosing will depend upon the pharmacokinetic parameters of the KIRHy targeting agent in the formulation used. Typically, a composition is administered until a dosage is reached that achieves the desired effect. The composition may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data.

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amount is well within the capability of those skilled in the art, especially in light of the detailed disclosure provided herein. For any compound used in the method of the invention, the therapeutically effective dose can be estimated initially from appropriate in vitro assays. For example, a dose can be formulated in animal models to achieve a circulating concentration range that can be used to more accurately determine useful doses in humans. For example, a dose can be formulated in animal models to achieve a circulating concentration range that includes the IC₅₀ as determined in cell culture (i.e., the concentration of the test compound which achieves a half-maximal inhibition of the protein's biological activity). Such information can be used to more accurately determine useful doses in humans.

A therapeutically effective dose refers to that amount of the compound that results in amelioration of symptoms or a prolongation of survival in a patient. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds which exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. See, e.g., Fingl et al., 1975, in “The Pharmacological Basis of Therapeutics”, Ch. 1 p. 1. Dosage amount and interval may be adjusted individually to provide plasma levels of the active moiety which are sufficient to maintain the desired effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compounds should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

An exemplary dosage regimen for polypeptides or other compositions of the invention will be in the range of about 0.01 μg/kg to 100 mg/kg of body weight daily, with the preferred dose being about 0.1 μg/kg to 25 mg/kg of patient body weight daily, varying in adults and children. Dosing may be once daily, or equivalent doses may be delivered at longer or shorter intervals.

The amount of composition administered will, of course, be dependent on the subject being treated, on the subject's age and weight, the severity of the affliction, the manner of administration and the judgment of the prescribing physician.

5.10.3 Routes of Administration

The route of administration of the pharmaceutical composition is in accord with known methods, e.g. orally, through injection by intravenous, intraperitoneal, intracerebral (intra-parenchymal), intracerebroventricular, intramuscular, intra-ocular, intra-arterial, intraportal, intralesional routes, intramedullary, intrathecal, intraventricular, transdermal, subcutaneous, intraperitoneal, intranasal, enteral, topical, sublingual, urethral, vaginal, or rectal means, by sustained release systems, by implantation devices, or through inhalation. Where desired, the compositions may be administered by bolus injection or continuously by infusion, or by implantation device.

Alternatively or additionally, the composition may be administered locally via implantation of a membrane, sponge, or another appropriate material on to which the KIRHy targeting agent has been absorbed or encapsulated. Where an implantation device is used, the device may be implanted into any suitable tissue or organ, and delivery of the KIRHy targeting agent may be via diffusion, timed-release bolus, or continuous administration.

In some cases, it may be desirable to use pharmaceutical compositions in an ex vivo manner. In such instances, cells, tissues, or organs that have been removed from the patient are exposed to the pharmaceutical compositions after which the cells, tissues and/or organs are subsequently implanted back into the patient.

In other cases, a KIRHy targeting agent can be delivered by implanting certain cells that have been genetically engineered to express and secrete the polypeptide. Such cells may be animal or human cells, and may be autologous, heterologous, or xenogeneic. Optionally, the cells may be immortalized. In order to decrease the chance of an immunological response, the cells may be encapsulated to avoid infiltration of surrounding tissues. The encapsulation materials are typically biocompatible, semi-permeable polymeric enclosures or membranes that allow the release of the protein product(s) but prevent the destruction of the cells by the patient's immune system or by other detrimental factors from the surrounding tissues.

5.11 Combination Therapy

KIRHy targeting agents of the invention can be utilized in combination with other therapeutic agents, and may enhance the effect of these other therapeutic agents such that a lesser daily amount, lesser total amount or reduced frequency of administration is required in order to achieve the same therapeutic effect at reduced toxicity. For cancer, these other therapeutics include, for example radiation treatment, chemotherapeutic agents, as well as other growth factors. For transplant rejection or autoimmune diseases, these other therapeutics include for example immunosuppressants such as cyclosporine, azathioprine corticosteroids, tacrolimus or mycophenolate mofetil.

In one embodiment, anti-KIRHy antibody is used as a radiosensitizer. In such embodiments, the anti-KIRHy antibody is conjugated to a radiosensitizing agent. The term “radiosensitizer,” as used herein, is defined as a molecule, preferably a low molecular weight molecule, administered to animals in therapeutically effective amounts to increase the sensitivity of the cells to be radiosensitized to electromagnetic radiation and/or to promote the treatment of diseases that are treatable with electromagnetic radiation. Diseases that are treatable with electromagnetic radiation include neoplastic diseases, benign and malignant tumors, and cancerous cells.

The terms “electromagnetic radiation” and “radiation” as used herein include, but are not limited to, radiation having the wavelength of 10-20 to 100 meters. Preferred embodiments of the present invention employ the electromagnetic radiation of: gamma-radiation (10⁻²⁰ to 10⁻¹³ m), X-ray radiation (10⁻¹² to 10⁻⁹ m), ultraviolet light (10 nm to 400 nm), visible light (400 nm to 700 nm), infrared radiation (700 nm to 1.0 mm), and microwave radiation (1 mm to 30 cm).

Radiosensitizers are known to increase the sensitivity of cancerous cells to the toxic effects of electromagnetic radiation. Many cancer treatment protocols currently employ radiosensitizers activated by the electromagnetic radiation of X-rays. Examples of X-ray activated radiosensitizers include, but are not limited to, the following: metronidazole, misonidazole, desmethylmisonidazole, pimonidazole, etanidazole, nimorazole, mitomycin C, RSU 1069, SR 4233, EO9, RB 6145, nicotinamide, 5-bromodeoxyuridine (BUdR), 5-iododeoxyuridine (IUdR), bromodeoxycytidine, fluorodeoxyuridine (FUdR), hydroxyurea, cisplatin, and therapeutically effective analogs and derivatives of the same.

Photodynamic therapy (PDT) of cancers employs visible light as the radiation activator of the sensitizing agent. Examples of photodynamic radiosensitizers include the following, but are not limited to: hematoporphyrin derivatives, Photofrin(r), benzoporphyrin derivatives, NPe6, tin etioporphyrin (SnET2), pheoborbide-a, bacteriochlorophyll-a, naphthalocyanines, phthalocyanines, zinc phthalocyanine, and therapeutically effective analogs and derivatives of the same.

Chemotherapy treatment can employ anti-neoplastic agents including, for example, alkylating agents including: nitrogen mustards, such as mechlorethamine, cyclophosphamide, ifosfamide, melphalan and chlorambucil; nitrosoureas, such as carmustine (BCNU), lomustine (CCNU), and semustine (methyl-CCNU); ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; ppipodophylotoxins such as etoposide and teniposide; antibiotics such as actimomycin D, daunomycin (rubidomycin), doxorubicin, mitoxantrone, idarubicin, bleomycins, plicamycin (mithramycin), mitomycinc, and actinomycin; enzymes such as L-asparaginase; biological response modifiers such as interferon-alpha, IL-2, G-CSF and GM-CSF; miscellaneous agents including platinum coordination complexes such as cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide.

Combination therapy with growth factors can include cytokines, lymphokines, growth factors, or other hematopoietic factors such as M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNF0, TNF1, TNF2, G-CSF, Meg-CSF, GM-CSF, thrombopoietin, stem cell factor, and erythropoietin. Other compositions can include known angiopoietins, for example, vascular endothelial growth factor (VEGF). Growth factors include angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor, cytokine-induced neutrophil chemotactic factor 1, cytokine-induced neutrophil chemotactic factor 2, endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor 2, growth related protein, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor, nerve growth factor, nerve growth factor receptor, neurotrophin-3, neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor, pre-B cell growth stimulating factor, stem cell factor, stem cell factor receptor, transforming growth factor, transforming growth factor 1, transforming growth factor 1.2, transforming growth factor 2, transforming growth factor 3, transforming growth factor 5, latent transforming growth factor 1, transforming growth factor binding protein I, transforming growth factor binding protein II, transforming growth factor binding protein III, tumor necrosis factor receptor type I, tumor necrosis factor receptor type II, urokinase-type plasminogen activator receptor, vascular endothelial growth factor, and chimeric proteins and biologically or immunologically active fragments thereof.

5.12 Diagnostic Uses of KIRHy

5.12.1 Assays for Determining KIRHy-Expression Status

Determining the status of KIRHy expression patterns in an individual may be used to diagnose cancer and may provide prognostic information useful in defining appropriate therapeutic options. Similarly, the expression status of KIRHy may provide information useful for predicting susceptibility to particular disease stages, progression, and/or tumor aggressiveness. The invention provides methods and assays for determining KIRHy expression status and diagnosing cancers that express KIRHy.

In one aspect, the invention provides assays useful in determining the presence of cancer in an individual, comprising detecting a significant increase or decrease, as applicable, in KIRHy mRNA or protein expression in a test cell or tissue or fluid sample relative to expression levels in the corresponding normal cell or tissue. In one embodiment, the presence of KIRHy mRNA is evaluated in tissue samples of a myeloproliferative disorder. The presence of significant KIRHy expression may be useful to indicate whether the disorder is susceptible to KIRHy targeting using a targeting composition of the invention. In a related embodiment, KIRHy expression status may be determined at the protein level rather than at the nucleic acid level. For example, such a method or assay would comprise determining the level of KIRHy expressed by cells in a test tissue sample and comparing the level so determined to the level of KIRHy expressed in a corresponding normal sample. In one embodiment, the presence of KIRHy is evaluated, for example, using immunohistochemical methods. KIRHy antibodies capable of detecting KIRHy expression may be used in a variety of assay formats well known in the art for this purpose.

Peripheral blood may be conveniently assayed for the presence of cancer cells, including lymphomas and leukemias, using RT-PCR to detect KIRHy expression. The presence of RT-PCR amplifiable KIRHy mRNA provides an indication of the presence of one of these types of cancer. A sensitive assay for detecting and characterizing carcinoma cells in blood may be used (Racila, et al., Proc. Natl. Acad. Sci. USA 95: 4589-4594 (1998), herein incorporated by reference in its entirety). This assay combines immunomagnetic enrichment with multiparameter flow cytometric and immunohistochemical analyses, and is highly sensitive for the detection of cancer cells in blood, reportedly capable of detecting one epithelial cell in 1 ml of peripheral blood.

A related aspect of the invention is directed to predicting susceptibility to developing cancer in an individual. In one embodiment, a method for predicting susceptibility to cancer comprises detecting KIRHy mRNA or KIRHy in a tissue sample, its presence indicating susceptibility to cancer, wherein the degree of KIRHy mRNA expression present is proportional to the degree of susceptibility.

Yet another related aspect of the invention is directed to methods for assessment of tumor aggressiveness (Orlandi, et al., Cancer Res. 62:567 (2002), herein incorporated by reference in its entirety). In one embodiment, a method for gauging aggressiveness of a tumor comprises determining the level of KIRHy mRNA or KIRHy protein expressed by cells in a sample of the tumor, comparing the level so determined to the level of KIRHy mRNA or KIRHy protein expressed in a corresponding normal tissue taken from the same individual or a normal tissue reference sample, wherein the degree of KIRHy mRNA or KIRHy protein expression in the tumor sample relative to the normal sample indicates the degree of aggressiveness.

Methods for detecting and quantifying the expression of KIRHy mRNA or protein are described herein and use standard nucleic acid and protein detection and quantification technologies well known in the art. Standard methods for the detection and quantification of KIRHy mRNA include in situ hybridization using labeled KIRHy riboprobes (Gemou-Engesaeth, et al., Pediatrics, 109:E24-E32 (2002)), Northern blot and related techniques using KIRHy polynucleotide probes (Kunzli, et al., Cancer 94:228 (2002)), RT-PCR analysis using primers specific for KIRHy (Angchaiskisiri, et al., Blood 99:130 (2002)), and other amplification type detection methods, such as, for example, branched DNA (Jardi, et al., J. Viral Hepat. 8:465-471 (2001)), SISBA, TMA (Kimura, et al., J. Clin. Microbiol. 40:439-445 (2002)), and microarray products of a variety of sorts, such as oligos, cDNAs, and monoclonal antibodies. In a specific embodiment, real-time RT-PCR may be used to detect and quantify KIRHy mRNA expression (Simpson, et al., Molec. Vision 6:178-183 (2000)). Standard methods for the detection and quantification of protein may be used for this purpose. In a specific embodiment, polyclonal or monoclonal antibodies specifically reactive with the wild-type KIRHy may be used in an immunohistochemical assay of biopsied tissue (Ristimaki, et al., Cancer Res. 62:632 (2002), herein incorporated by reference in its entirety).

5.12.2 Diagnostic Assays and Kits

The present invention further provides methods to identify the presence or expression of KIRHy, or homolog thereof, in a test sample, using a nucleic acid probe or antibodies of the present invention, optionally conjugated or otherwise associated with a suitable label.

In general, methods for detecting a KIRHy polynucleotide can comprise contacting a sample with a compound that binds to and forms a complex with the polynucleotide for a period sufficient to form the complex, and detecting the complex, so that if a complex is detected, a polynucleotide of the invention is detected in the sample. Such methods can also comprise contacting a sample under stringent hybridization conditions with nucleic acid primers that anneal to a polynucleotide of the invention under such conditions, and amplifying annealed polynucleotides, so that if a polynucleotide is amplified, a polynucleotide of the invention is detected in the sample.

In general, methods for detecting a polypeptide of the invention can comprise contacting a sample with a compound that binds to and forms a complex with the polypeptide for a period sufficient to form the complex, and detecting the complex, so that if a complex is detected, a polypeptide of the invention is detected in the sample.

In detail, such methods comprise incubating a test sample with one or more of the antibodies or one or more of the nucleic acid probes of the present invention and assaying for binding of the nucleic acid probes or antibodies to components within the test sample.

Conditions for incubating a nucleic acid probe or antibody with a test sample vary. Incubation conditions depend on the format employed in the assay, the detection methods employed, and the type and nature of the nucleic acid probe or antibody used in the assay. One skilled in the art will recognize that any one of the commonly available hybridization, amplification or immunological assay formats can readily be adapted to employ the nucleic acid probes or antibodies of the present invention. Examples of such assays can be found in Chard, T., An Introduction to Radioimmunoassay and Related Techniques, Elsevier Science Publishers, Amsterdam, The Netherlands (1986); Bullock, G. R. et al., Techniques in Immunocytochemistry, Academic Press, Orlando, Fla. Vol. 1 (1982), Vol. 2 (1983), Vol. 3 (1985); Tijssen, P., Practice and Theory of immunoassays: Laboratory Techniques in Biochemistry and Molecular Biology, Elsevier Science Publishers, Amsterdam, The Netherlands (1985). The test samples of the present invention include cells, protein or membrane extracts of cells, or biological fluids such as sputum, blood, serum, plasma, lymphatic fluid, or urine. The test sample used in the above-described method will vary based on the assay format, nature of the detection method and the tissues, cells or extracts used as the sample to be assayed. Methods for preparing protein extracts or membrane extracts of cells are well known in the art and can be readily be adapted in order to obtain a sample which is compatible with the system utilized.

In another embodiment of the present invention, kits are provided which contain the necessary reagents to carry out the assays of the present invention. Specifically, the invention provides a compartment kit to receive, in close confinement, one or more containers which comprises: (a) a first container comprising one of the probes or antibodies of the present invention; and (b) one or more other containers comprising one or more of the following: wash reagents, reagents capable of detecting presence of a bound probe or antibody.

In detail, a compartment kit includes any kit in which reagents are contained in separate containers. Such containers include small glass containers, plastic containers or strips of plastic or paper. Such containers allows one to efficiently transfer reagents from one compartment to another compartment such that the samples and reagents are not cross-contaminated, and the agents or solutions of each container can be added in a quantitative fashion from one compartment to another. Such containers will include a container which will accept the test sample, a container which contains the antibodies used in the assay, containers which contain wash reagents (such as phosphate buffered saline, Tris-buffers, etc.), and containers which contain the reagents used to detect the bound antibody or probe. Types of detection reagents include labeled nucleic acid probes, labeled secondary antibodies, or in the alternative, if the primary antibody is labeled, the enzymatic, or antibody binding reagents which are capable of reacting with the labeled antibody. One skilled in the art will readily recognize that the disclosed probes and antibodies of the present invention can be readily incorporated into one of the established kit formats which are well known in the art.

5.12.3 Medical Imaging

KIRHy antibodies that recognize KIRHy and fragments thereof are useful in medical imaging of sites expressing KIRHy. Such methods involve chemical attachment of a labeling or imaging agent, such as a radioisotope, which include ⁶⁷Cu, ⁹⁰Y, ¹²⁵I, ¹³¹I, ¹⁸⁶Re, ¹⁸⁸Re, ²¹¹At, ²¹²Bi, administration of the labeled antibody and fragment to a subject in a pharmaceutically acceptable carrier, and imaging the labeled antibody and fragment in vivo at the target site. Radiolabelled anti-KIRHy antibodies or fragments thereof may be particularly useful in in vivo imaging of KIRHy expressing cancers, such as lymphomas or leukemias. Such antibodies may provide highly sensitive methods for detecting metastasis of KIRHy-expressing cancers.

Upon consideration of the present disclosure, one of skill in the art will appreciate that many other embodiments and variations may be made in the scope of the present invention. Accordingly, it is intended that the broader aspects of the present invention not be limited to the disclosure of the following examples.

### 6. EXAMPLES

### Example 1

### Isolation of SEQ ID NO: 1 from a cDNA Library of Human Cells

The novel nucleic acids of SEQ ID NO: 1 were obtained from various human cDNA libraries using standard PCR, sequencing by hybridization sequence signature analysis, and Sanger sequencing techniques. The inserts of the library were amplified with PCR using primers specific for vector sequences flanking the inserts. These samples were spotted onto nylon membranes and interrogated with oligonucleotide probes to give sequence signatures. The clones were clustered into groups of similar or identical sequences, and single representative clones were selected from each group for gel sequencing. The 5′ sequence of the amplified inserts were then deduced using the reverse M13 sequencing primer in a typical Sanger sequencing protocol. PCR products were purified and subjected to fluorescent dye terminator cycle sequencing. Single-pass gel sequencing was done using a 377 Applied Biosystems (ABI) sequencer. These inserts was identified as a novel sequence not previously obtained from this library and not previously reported in public databases. This sequence is designated as SEQ ID NO: 1 in the attached sequence listing.

### Example 2

### Assemblage of SEQ ID NO: 2

The novel nucleic acids (SEQ ID NO: 2) of the invention were assembled from sequences that were obtained from various cDNA libraries by methods described in Example 1 above, and in some cases obtained from one or more public databases. The final sequence was assembled using the EST sequence as seed. Then a recursive algorithm was used to extend the seed into an extended assemblage, by pulling additional sequences from different databases (i.e. Hyseq's database containing EST sequences, dbEST, gb pri, and UniGene) that belong to this assemblage. The algorithm terminated when there was no additional sequences from the above databases that would extend the assemblage. Inclusion of component sequences into the assemblage was based on a BLASTN hit to the extending assemblage with BLAST score greater than 300 and percent identity greater than 95%.

TABLE 1

| | Corresponding | Corresponding | | | | |
|---|---|---|---|---|---|---|
| SEQ | SEQ ID NO. in | SEQ ID NO. in | | | Smith- | |
| ID | U.S.S.N. | U.S.S.N. | Accession | | Waterman | % |
| NO. | 09/631,451 | 09/491,404 | No. | Description | Score | Identity |
| 3 | 156 | 2882 | AJ010101 | Homo sapiens IRC1a | 334 | 37 |
| | | | | (NK cell IRC1a gene) | | |

### Example 3

### Tissue Expression Analysis of KIRHy1 Polynucleotides and Chromosomal Localization

By checking the Hyseq proprietary database established from screening by hybridization, SEQ ID NO: 2 was found to be expressed in following human tissue/cell cDNA:

TABLE 2

| | | Total No. | |
|---|---|---|---|
| Library | No. of Positive | of Clones | |
| Name | Clones | in the Library | Tissue Origin |
| LUC001 | 26 | 210372 | Leukocytes |
| ASP001 | 3 | 32114 | Adult spleen |
| LUC003 | 2 | 30296 | Leukocytes |
| SPLc01 | 6 | 110573 | Spleen |
| FLG001 | 1 | 28154 | Whole organ |
| ALG001 | 1 | 28271 | Adult lung |
| CLN001 | 1 | 28708 | Colon |
| UTR001 | 1 | 29595 | Uterus |
| ABR001 | 1 | 30163 | Adult brain |
| BMD002 | 2 | 75816 | Bone marrow |
| LGT002 | 4 | 158948 | Lung tumor |
| PLA003 | 1 | 80877 | Placenta |
| DGD004 | 1 | 91423 | Lymphocytes/Myeloma |
| THMc02 | 1 | 96791 | Thymus |
| SIN001 | 1 | 142562 | Whole organ |
| STM001 | 1 | 181899 | Bone Marrow |
| AOV010 | 1 | 259409 | Ovary |
| FLS002 | 1 | 709733 | Fetal liver/spleen |

The gene corresponding to SEQ ID NO: 2 was mapped to human chromosome 17 (specifically 17q25.2) by BLAST analysis with human genome sequences.

### Example 4

### KIRHy1 mRNA is Highly Expressed in B-Cell Cell Lines and Tissues

FIG. 8 shows the relative expression of mRNA derived from B-cell cell lines, healthy tissues, and tumor tissues derived from B cell lymphomas, follicular lymphomas, and myelomas.

Total mRNA derived from tissues and cells lines was subjected to quantitative real-time PCR (TaqMan) (Simpson, et al., Molec. Vision, 6:178-183 (2000) herein incorporated by reference) to determine the relative expression of KIRHy mRNA. Total mRNA derived from cell lines (obtained from ATCC, Manassas, Va.) was isolated using standard protocols. The cell lines were derived from acute myelogenous leukemia (AML193), acute myeloid leukemia (AM565), acute myelogenous leukemia (KG1), anaplastic large T cell lymphoma (L5664), B cell lymphoma (RA1), chronic myelogenous leukemia (K562), diffuse large B cell lymphoma (L22601), follicular lymphoma grade II/III (L5856), histiocytic lymphoma (U937), Hodgkin's lymphoma (HD5664), large B cell lymphoma (DB), non-Hodgkin's lymphoma (RL), and plasmacytoma (RPMI).

The mRNA derived from the tumor tissues was prepared from malignant B cells that had been isolated from the tumors. Tumor samples were obtained from different patients suffering from B cell lymphomas (H02-85T, H02-86T, H02-87T, H02-88T, H02-89T), follicular lymphoma (H02-74T, H02-75T, H02-76T, H02-77T, H02-78T), and myeloma (H02-79T, H02-80T, H02-81T, H02-82T, H02-83T, H02-84T). DNA sequences encoding Elongation Factor 1 were used as a positive control and normalization factors in all samples. All assays were performed in duplicate with the resulting values averaged. The y-axis shows the relative expression of the KIRHy1 mRNA, wherein the lowest expression was set as equal to 1 and the rest of the values are expressed as relative to 1.

FIG. 8 shows that relatively little expression of the KIRHy1 gene was found in healthy tissues with the exception of tissues that either produce or are infiltrated by B lymphocytes, namely lymph node and small intestine. The results show that KIRHy1 is most highly upregulated in AML and histiocytic lymphoma, indicating that KIRHy1 may be used as a therapeutic target or as a diagnostic marker for these types of disorders.

### Example 5

### Expression of KIRHy1 Proteins

A. Expression of 10458-V5-His and 10458-WT Protein in E. coli

1. 10458-V5-His and 10458-WT E. coli Expression Constructs

The DNA sequence encoding the extracellular domain of KIRHy1 spanning nt 52-474 (SEQ ID NO: 52) was amplified by PCR using the following primers: 5′-atgattgtcactcaaatcaccggtc-3′ (SEQ ID NO: 53) and 5′-ttattagccggtcagagttggggagctg-3′ (SEQ ID NO: 54). The PCR product was cloned in the E. coli expression vector pCRT7-CT-TOPO (invitrogen), generating the expression construct pCRT7-10458-WT.

The 10458-V5-His E. coli expression construct pCRT7-10458-V5-His was generated in a similar fashion using the following primers: 5′-atgattgtcactcaaatcaccggtc-3′ (SEQ ID NO: 55) and 5′-gccggtcagagttggggagctg-3′ (SEQ ID NO: 56).

2. 10458-V5-his and 10458-WT Protein Expression in E. Coli

The pCRT7-10458-V5-His (or pCRT-10458-WT) plasmid was transformed into BL21 (or DE3) pLysS competent E. coli cells (invitrogen). A colony was grown overnight in Terrific Broth (Difco) containing 50 μg/ml carbenicillin in a 37° C. shaker. The overnight culture was diluted 1:40 into fresh Terrific Broth containing 50 μg/ml carbenicillin in a 37° C. shaker set at 300 rpm. When the culture reached OD₆₀₀ of 1-2, IPTG was added to a final concentration of 1 mM to induce recombinant protein expression. Three hours after induction, the E. coli cell paste was harvested by centrifugation at 9000 rpm for 30 min at 4° C. and stored at −20° C. until used for purification (infra).

B. Expression in Mammalian Tissue Culture Cells

1. Expression of 10458-Fc Fusion Protein (SEQ ID NO: 31)

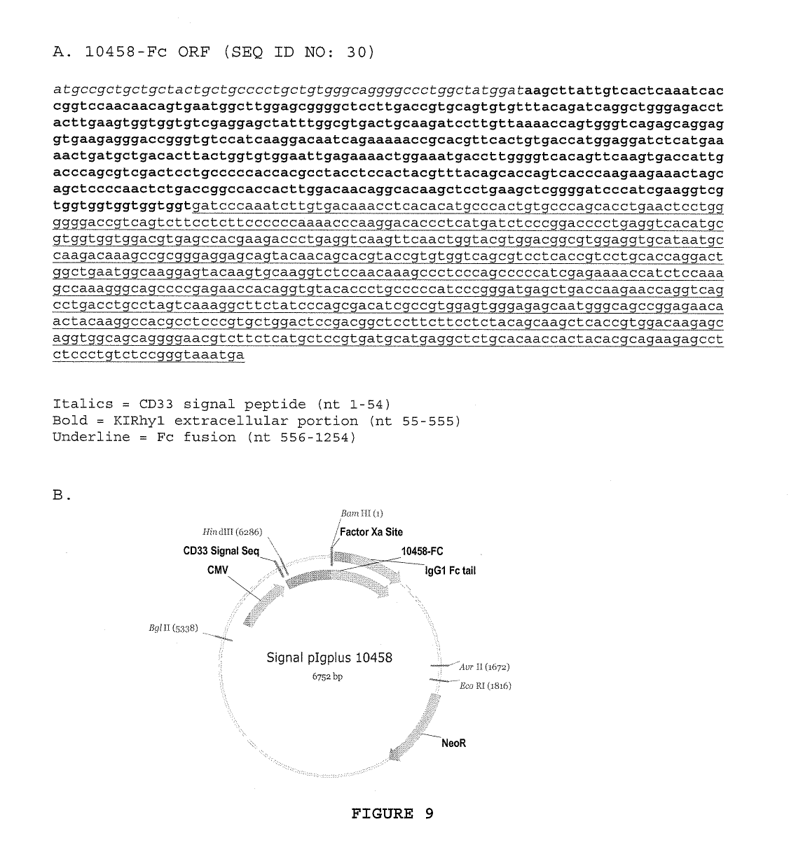

To ensure generation of antibodies against the native KIRHy1 molecule, the Signal pip Plus vector system was used (Ingenius, R&D Systems Europe Ltd., Abingdon, Oxfordshire, U.K.). The Signal pig Plus vector contains the signal peptide of CD33 and the human IgG1 Fc fragment. SEQ ID NO: 30 represents the open reading frame (ORF) for the KIRHy1-Fc (herein denoted as 10458-Fc) fragment (see FIG. 9). The CD33 signal peptide (italics) spans nt 1-54 of SEQ ID NO: 30, the KIRHy1 extracellular domain (residues 18-150 of SEQ ID NO: 3) (bold) spans nt 55-555 of SEQ ID NO: 30), and the IgG1 Fc fragment (underline) spans nt 556-1254 of SEQ ID NO: 30.

0.3×10⁶ CHO cells (ATCC) were plated in 6-well plates the day before transfection. Cells were transfected with 2 μg/well of 10458-Fc expression plasmid plus 6 μl of Fugene-6 (Roche). 48 hours post-transfection, the standard medium (F12, Invitrogen) was replaced with media containing G418 (Invitrogen) at 1 mg/ml. The media was changed every other day for 2 weeks to select for positive cells (those containing the 10458-Fc plasmid). After 2 weeks, cells were harvested and the cell media was assayed by Western blot for expression of 10458-Fc.

Stable 10458-Fc fusion protein-expressing CHO cells were grown and expanded in F-12K Nutrient Mixture (Kaighn's Modification, Invitrogen) medium plus 10% FBS, L-glutamine and penicillin/streptomycin in the presence of 1 mg/ml gentamycin (G418). The cells were then suspension adapted. These suspension-adapted cells were further adapted to CHO-S-SFM II medium (Invitrogen) plus 0.5% FBS in the presence of G418. These suspension and serum-deficient adapted cells were further expanded and grown in spinners with rotation (100 rpm/min) at a cell density>1×10⁶ cells/ml. After growing for 7-8 days, the cell suspension was harvested and centrifuged at 3,000 rpm/min. The supernatant was collected and used for protein purification (infra).

2. Expression of 10458-V5-His Fusion Protein (SEQ ID NO: 58)

To generate the 10458-V5-His fusion protein, HEK-293 cells (ATCC) were stably transfected with the 10458-V5-His expression plasmid as described above for 10458-Fc. Cells were transfected with 2 μg/well of 10458-pintron-V5-His tagged C-terminal plasmid plus 6 μl of Fugene-6 (Roche). 48 hours post-transfection, the standard medium (DMEM, Invitrogen) was replaced with media containing G418 (Invitrogen) at 1 mg/ml. The media was changed every other day for 2 weeks to select for positive cells (those containing the 10458-V5-His plasmid). After 2 weeks, cells were harvested and the cell media was assayed by Western blot for expression of 10458-V5-His.

As described above for 10458-Fc stable cell lines, stable 10458-V5His fusion protein-expressing HEK-293 cells were grown in DMEM plus 10% FBS, L-glutamine, penicillin/streptomycin and G418 and were suspension- and serum-deficient adapted to FreeStyle 293 Expression Medium (Invitrogen) plus 0.5% FBS in the presence of G418. These adapted cells were further expanded, and the supernatant was collected for protein purification as described above.

### Example 6

### Purification of KIRHy1 Proteins

A. Purification of 10458-V5-His and 10458-WT Protein from E. coli

1. 10458-V5-His from E. coli

15 g E. coli cell paste containing 10458 V5-His protein was resuspended in 150 mL 25 mM Hepes, pH 7. To prevent the degradation during the purification process, a protease inhibitor cocktail for purification of His-tagged proteins (Sigma) was added at a ratio of 1 mL per 20 g cell paste. Cells were lysed by passage through a homogenizer at 18000-20000 psi. The whole cell lysate was spun at 14,300×g for 40 minutes to separate supernatant and pellet. The soluble supernatant was removed and the pellet (insoluble inclusion body) was kept. The pellet was washed with 25 mM Hepes, 0.5M NaCl, pH 7 buffer, after which it was solubilized with 25 mM Hepes, 6M Guanidine HCl, pH 7 buffer at 4° C. overnight to extract the proteins from the inclusion body. The protein solution was passed through a Zeta Plus BioCap 30 SP filter capsules (Cuno) to remove insoluble components, nucleic acid, cell debris and endotoxins.

A 10 mL HiTrap Ni-chelating affinity column (Amersham) was equilibrated with 25 mM Hepes, pH 7, 6M Guanidine HCl. The Guanidine HCl solubilized protein solution was filtered with a 0.22 μm PES filter and loaded onto a Ni-chelating affinity column. The Ni Column was washed with 20 mM imidazole for 10 Column Volumes (CV) and protein was eluted with a gradient of 20 mM to 500 mM imidazole over 35 CV. The fractions were analyzed by SDS-PAGE and Western blot using an anti-V5 antibody (Invitrogen). Fractions containing 10458 V5-His were pooled to yield a protein solution that was 90% pure when analyzed by Comassie staining of an SDS-gel. The pooled protein solution was concentrated to 1-2 mg/mL and dialyzed against 25 mM Hepes, 6M urea, pH 7 which is the storage buffer. The final protein solution was passed through a sterile 0.22 μm filter and stored at −80° C. The purified 10458 V5His protein from E. coli was used to immunize the mice for monoclonal antibody development.

2. 10458 WT from E. coli

The procedure for resuspension of cell paste, cell breakage and spin separation of the soluble supernatant and insoluble inclusion body are the same as described above. The soluble supernatant was removed and the pellet (insoluble inclusion body) was kept and washed with 25 mM Hepes, 1 M urea, pH 7 buffer, after which the remaining pellet was solubilized with 100 mL 25 mM Hepes, 6M urea, pH 7 buffer at 4° C. overnight. The solubilized protein solution was passed through a Zeta Plus BioCap 30 SP filter capsules (Cuno) and followed by passing through a 0.22 μm PES filter.

The filtered protein solution was concentrated to 2-3 mg/mL for further purification using Bio-Rad Preparative Electrophoresis apparatus. The 10458 WT protein was purified on a 14% Acrylamide preparative SDS gel to separate from other different size impurities. The collected fractions were analyzed with SDS-PAGE and western blot. Fractions containing 10458 V5 were pooled to yield a protein solution with 90% purity. The purified 10458 WT protein from E. coli was used for ELISA screening of the positive clones after fusion of spleen cells.

B. Purification of 10458-Fc and 10458 V5-His Protein from Mammalian Cells

1. Purification of 10458-Fc from CHO Cells

The extracellular domain of the 10458-Fc fusion protein (SEQ ID NO: 31) was expressed in CHO cells grown in CHO SFM II media with addition of 0.5% FBS. The cell culture was harvested and the supernatant was stored at −80° C.

The frozen culture supernatant (−80° C.) containing secreted 10458-Fc fusion protein was thawed at 4° C. Mammalian protease inhibitor cocktail (Sigma) was added at 1:500 (v/v) dilution. The 5 L supernatant was filtered through a 0.22 μm PES filter (Corning) and loaded onto a 5 mL protein A column (Amersham, Hitrap column) which was equilibrated with PBS buffer (GIBCO). After loading, the protein A column was washed with 10 column volume (CV) of PBS buffer. 10458-Fc protein was eluted with 8 CV of 0.1 M Glycine, pH 2.8 buffer and collected in 1 mL fractions. A calculated amount of 1 M Tris pH 9 buffer was pre-added in the collected fractions to immediately neutralize the pH 2.8 protein solution to pH 7 as soon as eluted from the column. The fractions were analyzed by SDS-PAGE and Western blot. Fractions containing 10458-Fc were pooled and analyzed to be 75-80% pure. The purified 10458-Fc protein from CHO cells was used to immunize mice for monoclonal antibody development (infra).

2. Purification of 10458-V5 from 293 HEK Cells

The extracellular domain of 10458 V5-His tagged protein (SEQ ID NO: 58) was expressed in 293 HEK cells grown in 293 free-style media with addition of 0.5% FBS. The cell culture was harvested and the supernatant was stored at −80° C.

The frozen culture supernatant (−80° C.) containing secreted 10458 V5-His protein was thawed at 4° C. Protease inhibitors EDTA and Pefbloc (Roche) were added to the media to a final concentration of 1 mM and 0.4 mM, respectively. The media was filtered through a 0.22 μm PES filter (Corning). Media was concentrated down to 10-fold using TFF system (Pall Filtron) with 10 kDa cut-off membrane. The concentrated media was buffer exchanged with 20 mM sodium phosphate, 0.5M NaCl, pH 7. After media concentration/diafiltration, mammalian protease inhibitor cocktail (Sigma) was added at 1:500 (v/v) dilution.

A HiTrap Ni-chelating affinity column (Pharmacia) was equilibrated with 20 mM sodium phosphate, pH 7, 0.5 M NaCl. The buffer-exchanged media was filtered with 0.22 μm PES filter and loaded onto Ni-chelating affinity column. The Ni column was washed with 20 mM imidazole for 15 Column Volume (CV) and protein was eluted with a gradient of 20 mM to 220 mM imidazole over 30 CV. The fractions were analyzed by SDS-PAGE and Western blot. Fractions containing 10458 V5-His were pooled and analyzed to be 85% pure. The pooled protein solution was concentrated to 1 mg/mL and dialyzed against 20 mM sodium phosphate, 0.15M NaCl, pH 7 (PBS). The final protein solution in PBS buffer was passed through a sterile 0.22 μm filter and stored at −80° C. The purified 10458 V5-His protein from 293 HEK cells was used for ELISA screening of the positive clones after fusion of spleen cells for mAb generation.

### Example 7

### Production of KIRHy-Specific Antibodies

A. Polyclonal Antibodies

Cells expressing KIRHy are identified using KIRHy antibodies. Polyclonal antibodies are produced by DNA vaccination or by injection of peptide antigens into rabbits or other hosts. An animal, such as a rabbit, is immunized with a peptide from the extracellular region of KIRHy conjugated to a carrier protein, such as BSA (bovine serum albumin) or KLH (keyhole limpet hemocyanin). The rabbit is initially immunized with conjugated peptide in complete Freund's adjuvant, followed by a booster shot every two weeks with injections of conjugated peptide in incomplete Freund's adjuvant. Anti-KIRHy antibody is affinity purified from rabbit serum using KIRHy peptide coupled to Affi-Gel 10 (Bio-Rad), and stored in phosphate-buffered saline with 0.1% sodium azide.

One such polyclonal antibody was made using KLH conjugated to an immunogenic KIRHy peptide having the amino acid sequence Glu-Glu-Pro-Thr-Glu-Tyr-Ser-Thr-Ile-Ser-Arg-Pro (SEQ ID NO: 11) that corresponds to amino acid residues 294-305 of SEQ ID NO: 3 and is also found in the C-terminal tails of KIRHy3, KIRHy6 and KIRHy7. The anti-KIRHy peptide polyclonal antibody is herein denoted as 10458a. To determine that 10458a was KIRHy-specific, an expression vector encoding a V5/His tagged-KIRHy1 (pintron-KIRHy1, Nuvelo Inc.) was introduced into mammalian COS-7 cells. Western blot analysis of protein extracts of non-transfected cells and the KIRHy-containing cells was performed using 10458a as the primary antibody and a horseradish peroxidase-labeled anti-rabbit antibody (donkey anti-rabbit IgG) as the secondary antibody. Detection of an approximately 48 kD band in the KIRHy1-containing cells and lack thereof in the control cells indicated that 10458a was specific for KIRHy recognizing KIRHy1 as well as KIRHy3, KIRHy6, and KIRHy7.

B. Monoclonal Antibodies

1. mAb Generated from Bacterial Protein

Monoclonal antibodies were produced by injecting mice with a bacterially produced KIRHy1 extracellular peptide (see Example 5A supra), using standard protocols. The mice were boosted every 2 weeks until an appropriate immune response was identified. After fusion of the murine splenocytes with murine myeloma cells, the resulting hybridomas were grown in culture and selected for antibody production by clonal selection. The anti-KIRHy1 antibodies were secreted into the culture supernatant, and screened by enzyme-linked immunosorbent assay (ELISA) and Western blot analysis.

One of the monoclonal antibodies (mAb) that elicited a strong response specific to KIRHy1 was Clone #20 (an IgM), the characterization of which is described below in Example 7.

2. mAb Generated from 10458-Fc Fragment

Mice were injected s.c., 5 times at 2-3 week intervals with 0.05 mg 10458-Fc in phosphate buffered saline, pH 7.4. Two mice were injected, via a combination of s.c, i.p. and i.v. routes, with 0.1 to 0.3 mg of 10458-Fc each day for 3 days prior to the fusion. Cells from the spleen and lymph nodes of the mice were isolated and fused with P3X63-Ag8.653 myeloma cells using 50% polyethylene glycol. Cells were cultured and a hybridoma library of HAT-selected cells were isolated essentially as described in Kenney, et al. (Biotechnology 13:787-90 (1995) herein incorporated by reference in its entirety). The hybridoma library was cloned using a fluorescent activated cell sorter with an automatic cell deposition unit. Single viable cells were sorted into 96-well plates based upon the analysis criteria of forward-scatter, side-scatter and propidium iodide fluorescence (Kenney, et. al. supra). Sera and supernatant were screened by ELISA using goat anti-mouse IgG (gamma chain-specific) antibody-coated ELISA plate wells, which were then incubated with the mouse anti-10458-Fc antibody containing serum or supernatant, followed by purified 10458-V5-His protein, followed by goat anti-V5 antibody-HRP conjugate, followed by TMB substrate solution and stop reagent. The plate wells were washed to remove unbound antibody or antigen between all incubations.

3. Humanized mAbs

Humanized monoclonal antibodies are produced either by engineering a chimeric murine/human monoclonal antibody in which the murine-specific antibody regions are replaced by the human counterparts and produced in mammalian cells, or by using transgenic “knock out” mice in which the native antibody genes have been replaced by human antibody genes and immunizing the transgenic mice as described above.

### Example 8

### Diagnostic Methods Using KIRHy-Specific Antibodies to Detect KIRHy Expression

Expression of KIRHy1 in leukemia and myeloma cell lines was detected by Western blot analysis using the anti-KIRHy peptide polyclonal antibody 10458a (see Example 6 for Western details). All AML samples and the histiocytic cell line were positive for KIRHy expression (see Table 3).

TABLE 3

| Cell Lines | Tissue of Origin | KIRHy Expression |
|---|---|---|
| Lymphoid Cells | | |
| CA46 | B lymphocyte, Burkitt's lymphoma | − |
| Daudi | B lymphoblast, Burkitt's lymphoma | +/− |
| GA10 | B lymphocyte, Burkitt's lymphoma | − |
| HT | B lymphoblast, diffuse mixed lymphoma | − |
| Jurkat | Acute T cell leukemia | − |
| Molt 4 | T lymphoblast, acute lymphoblastic leukemia | − |
| Ramos | B lymphocyte, Burkitt's lymphoma (American) | − |
| RL | ascites, B lymphoblast, non-Hodgkin's lymphoma | − |
| RPMI 8226 | peripheral blood, B lymphocyte, plasmacytoma, | +/− |
| | myeloma | |
| U266 | B lymphocyte, plasmacytoma, myeloma | + |
| Myeloid Cells | | |
| AML-193 | peripheral blood, monocyte, acute monocytic leukemia | ++++ |
| | (AML FAB M5) | |
| CTV-1 | human acute myeloid leukemia (AML FAB M5) | − |
| GDM-1 | acute myelomonoblastic leukemia | +++ |
| HL60 | acute promyelocytic leukemia | + |
| Kasumi-3 | peripheral blood, lymphoblast, acute myeloblastic | +++ |
| | leukemia | |
| KG-1 | bone marrow, acute myelogenous leukemia | ++++ |
| K-562 | chronic myelogenous leukemia | − |
| ML-2 | human acute myelomonocytic leukemia (AML FAB M4) | + |
| NB-4 | acute promyelocytic leukemia | + |
| OCI-AML2 | human acute myeloid leukemia (AML FAB M4) | +++ |
| THP-1 | peripheral blood, monocyte, acute monocytic leukemia | +/− |
| UT-7 | human acute myeloid leukemia (AML FAB M7) | ++ |
| U937 | histiocytic lymphoma | ++++ |

The results show that KIRHy is highly expressed in acute myelogenous leukemia (AML) and histiocytic lymphoma. In addition, these results are consistent with the relative expression of KIRHy mRNA (see Example 4), indicating that KIRHy1 targeting may be useful as a therapeutic treatment or diagnostic assay for these disorders.

Expression of KIRHy in tissue samples (normal or acute myelogenous leukemia (AML) bone marrow) was detected using the anti-KIRHy peptide polyclonal antibody, 10458a (see Example 6). Samples were prepared for immunohistochemical (IHC) analysis (LifeSpan Biosciences, Inc., Seattle, Wash.) by fixing the tissue in 10% formalin embedding in paraffin, and sectioning using standard techniques. Sections were stained using 10458a followed by incubation with a secondary horseradish peroxidase (HRP)-conjugated antibody and visualized by the product of the HRP enzymatic reaction. Data as seen in Table 4 shows that KIRHy is highly expressed on the cell surface of AML bone marrow tissues (in 5 out of 5 patient samples). No expression of KIRHy was observed on the cell surface of normal bone marrow samples (5 out of 5 patient samples). These data show that KIRHy expression is found in AML tissues and are consistent with the relative expression of KIRHy mRNA (see Example 4).

TABLE 4

| Tissue | Positive | Total |
|---|---|---|
| Acute myelogenous leukemia bone | 5 | 5 |
| marrow | | |
| Normal bone marrow | 0 | 5 |

Additionally, antibody blocking assays were performed and analyzed by IHC. Normal and AML bone marrow tissue samples were prepared for IHC as stated above; however, before the samples were incubated with 10458a, 10458a was pre-treated with an excess of SEQ ID NO: 11 and then processed as stated above. In all the samples tested (5 out of 5), SEQ ID NO: 11 blocked the reactivity of 10458a in the AML bone marrow samples (see Table 5).

TABLE 5

| Tissue | Positive | Total |
|---|---|---|
| AML bone marrow | 5 | 5 |
| AML bone marrow blocked with SEQ ID NO: 11 | 0 | 5 |
| Normal bone marrow | 0 | 5 |

Expression of KIRHy on the surface of cells within a blood sample is detected by flow cytometry. Peripheral blood mononuclear cells (PBMC) are isolated from a blood sample using standard techniques. The cells are washed with ice-cold PBS and incubated on ice with a KIRHy-specific polyclonal antibody for 30 min. The cells are gently pelleted, washed with PBS, and incubated with a fluorescent anti-rabbit antibody for 30 min. on ice. After the incubation, the cells are gently pelleted, washed with ice cold PBS, and resuspended in PBS containing 0.1% sodium azide and stored on ice until analysis. Samples are analyzed using a FACScalibur flow cytometer (Becton Dickinson) and CELLQuest software (Becton Dickinson). Instrument settings are determined using FACS-Brite calibration beads (Becton-Dickinson).

Tumors expressing KIRHy are imaged using KIRHy-specific antibodies conjugated to a radionuclide, such as ¹²³I, and injected into the patient for targeting to the tumor followed by X-ray or magnetic resonance imaging.

### Example 9

### Analysis of Clone #20 Monoclonal Antibody

A. Epitope Mapping

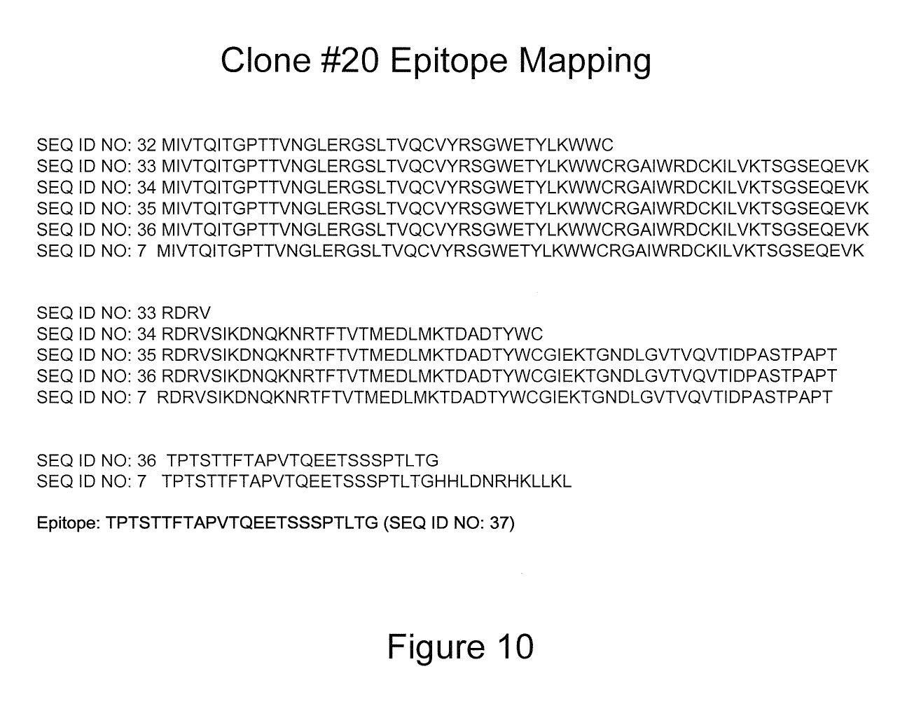

To determine the KIRHy epitope recognized by Clone #20, Western blot analysis was performed on a series of deletions of the KIRHy1 extracellular domain (SEQ ID NO: 7). The fragments were cloned into pCRT7 (Invitrogen) and expressed in and purified from E. coli (see Example 5A). The deletion fragments were resolved on a 4-20% polyacrylamide gel under denaturing conditions and probed with the Clone #20 supernatant using goat anti-mouse Ig-HRP as the secondary antibody. Clone #20 recognized the wild-type KIRHy1, V5/His-tagged KIRHy1 and KIRHy1 extracellular fragment 5 (SEQ ID NO: 36), but not fragments 1-4 (SEQ ID NO: 32-35) (see FIG. 10). Therefore, Clone #20 anti-KIRHy antibody recognizes the epitope between fragments 4 and 5, namely TPTSTTFTAPVTQEETSSSPTLTG (SEQ ID NO: 37).

B. FACS (Fluorescence Activated Cell Sorting) Analysis

1. Cell Surface Reactivity

OCI-AML-2 cells were used in a FACS analysis to determine if Clone #20 binding could shift the migration of the cells from negative with the isotype control (i.e. no cell surface binding) to positive (i.e. Clone #20 bound to cell surface KIRHy antigen).

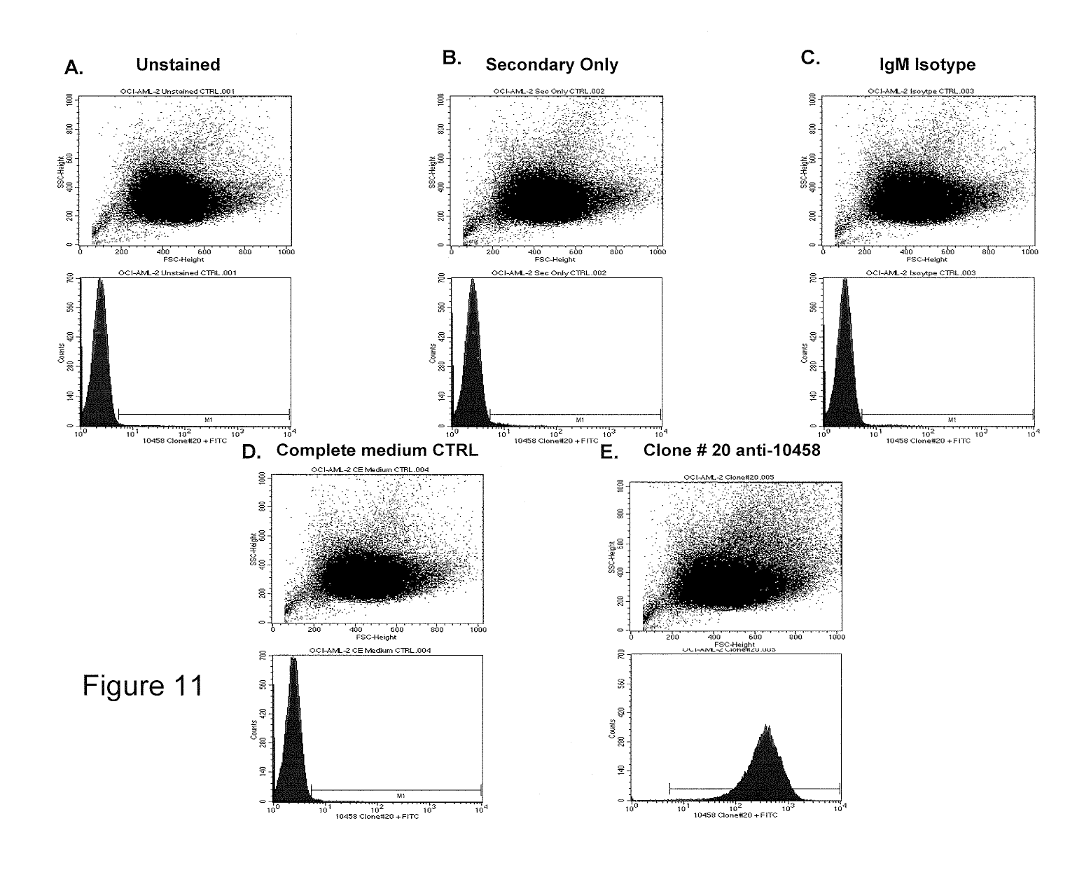

Briefly, 1-2×10⁶ target OCI-AML-2 cells were aliquoted, pelleted, and resuspended in 100 μl of FACS buffer (1% BSA in PBS) to which 1 μg/10⁶ cells of primary antibody (Clone #20 mAb, medium alone, IgM isotype control, or secondary antibody only) was added and incubated for 20 min on ice. Excess antibody was removed by twice washing in FACS wash buffer (PBS). The cell pellet was resuspended in 100 μl FACS buffer and secondary antibody (goat anti-mouse IgG+IgM (H+L)-FITC) was added if necessary. Excess secondary antibody was removed by twice washing in FACS wash buffer and the pellet was resuspended in 500 μl FACS wash buffer and analyzed by FACS. As can be seen in FIG. 11, only the anti-KIRHy mAb Clone #20 was able to label the surface of the target cells.

2. Epitope Blocking

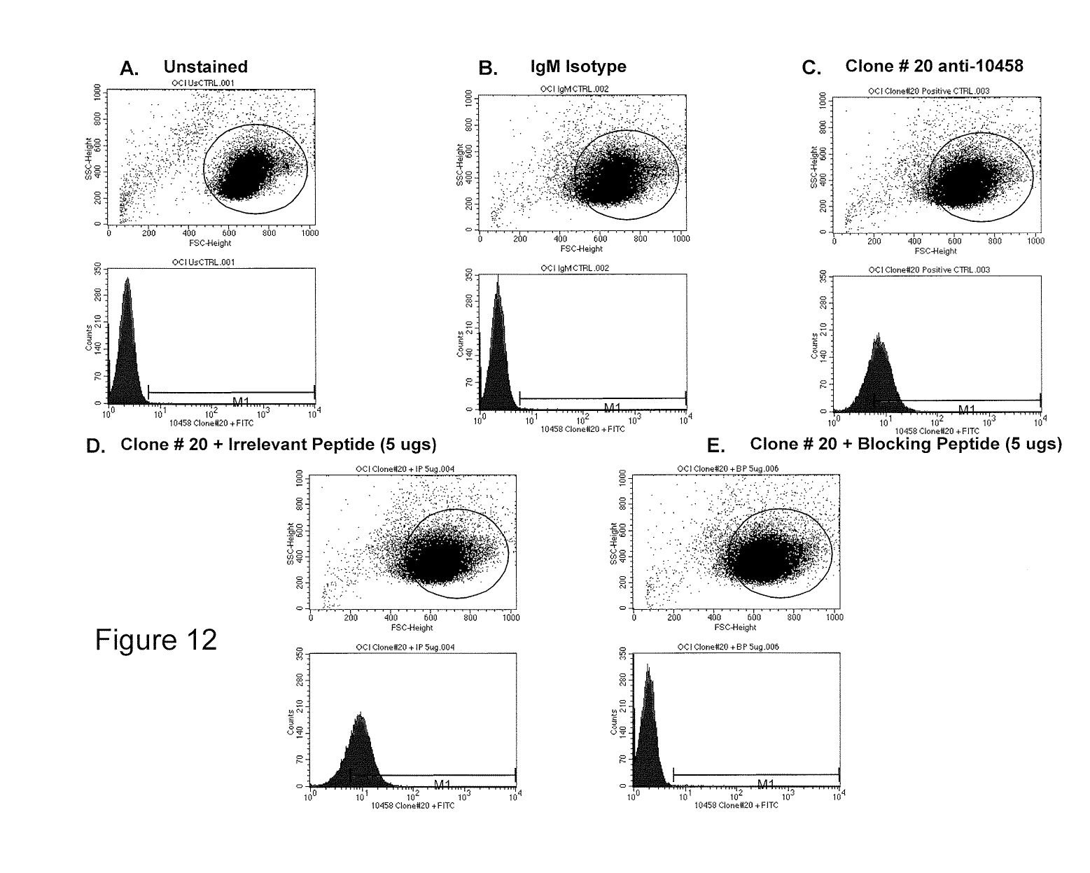

FACS analysis was utilized to determine if the Clone #20 mAb epitope [SEQ ID NO: 37, generated by SynPep Corp. (Dublin, Calif.)] could compete for the binding of Clone #20 mAb on the surface of OCI-AML-2 cells. Target cells were prepared as above with the exception of the addition of 5 μg of either the Clone #20 epitope (SEQ ID NO: 37) or an irrelevant peptide not found in the KIRHy1 sequence concomitant with the addition of primary antibody. As can be seen in FIG. 12, mAb Clone #20 shifts the cells indicating cell surface binding (FIG. 12C). Addition of the irrelevant peptide does not alter the shift (FIG. 12D); however, addition of the Clone #20 epitope peptide competes for binding to the cell surface and the cells do not shift (FIG. 12E).

3. Screening of AML Cell Lines

FACS analysis was performed as described above on a variety of AML cell lines. AML is most frequently sub-classified into eight well-defined variants, M0-M7 (see Table 6).

TABLE 6

| AML Stage | Classification |
|---|---|
| M0 | Acute myeloid leukemia with minimal |
| | evidence of myeloid differentiation |
| M1 | Acute myeloblastic leukemia without |
| | maturation |
| M2 | Acute myeloblastic leukemia with maturation |
| M3 | Acute promyelocytic leukemia (APL) |
| M4 | Acute myelomonocytic leukemia |
| M5 | Acute monocytic/monoblastic leukemia |
| M6 | Acute erythroleukemia |
| M7 | Acute megakaryoblastic leukemia |

Cell lines from the various AML stages were also analyzed. As can be seen in Table 7, many of the AML stages were positive for Clone #20 as were cell lines of chronic myeloid leukemia and T cell lymphoma origin. Therefore, anti-KIRHy antibodies are useful to target many stages of AML as well as other diseases.

TABLE 7

| | | KIRHy Expression |
|---|---|---|
| Cell Line | Histology | (FACS and Western) |
| HEK-293 | Neural | Negative |
| OCI-AML-2 | AML: M4 | Positive |
| NB4 | AML: M3 | Positive |
| ML-2 | AML: M4 | Positive |
| U937 | AML: ? | Positive |
| CTV-1 | AML: M5 | Positive (low) |
| HL-60 | AML: M2 | Positive |
| AML-193 | AML: M5 | Positive |
| MOLM-13 | AML: M5 | Positive |
| MonoMacs-6 | AML: M5 | Positive |
| THP-1 | AML: M? | Positive |
| KG-1 | AML: M? | Positive |
| MEG-1 | AML: Megakaryoblastic | Positive |
| Ku812 | CML: Basophil | Positive |
| SKM1 | AML: M5 | Positive |
| Kasumi | AML: M2 | Positive |
| Hut-78 | T cell lymphoma | Positive |
| Kasumi-6 | AML: M2 | Positive |
| B1647 | AML: erythromegakaryocytic | Positive |
| GDM-1 | AML: M? | Positive |
| ME-1 | AML: M4eo | Positive (low) |
| OCI-AML-5 | AML: M4 | Positive |
| F-36P | AML: M6 | Negative |
| PL-21 | AML: M? | Positive |
| NOMO-1 | MAL: M5a | Positive |
| GF-D8 | AML: M? | Positive |
| UT-7 | AML: M7 | Positive (low) |

C. Confocal Analysis

OCI-AML-2 samples were analyzed by confocal microscopy to identify binding of Clone #20 mAb to cell-surface KIRHy antigen. Cells were processed for FACS analysis as described above with the exception of the following steps. After removal of excess secondary antibody, cells were fixed in 4% formalin and placed on a glass microscope slide and then a non-bleaching reagent containing DAPI (to stain the nuclei) was added to the sample.

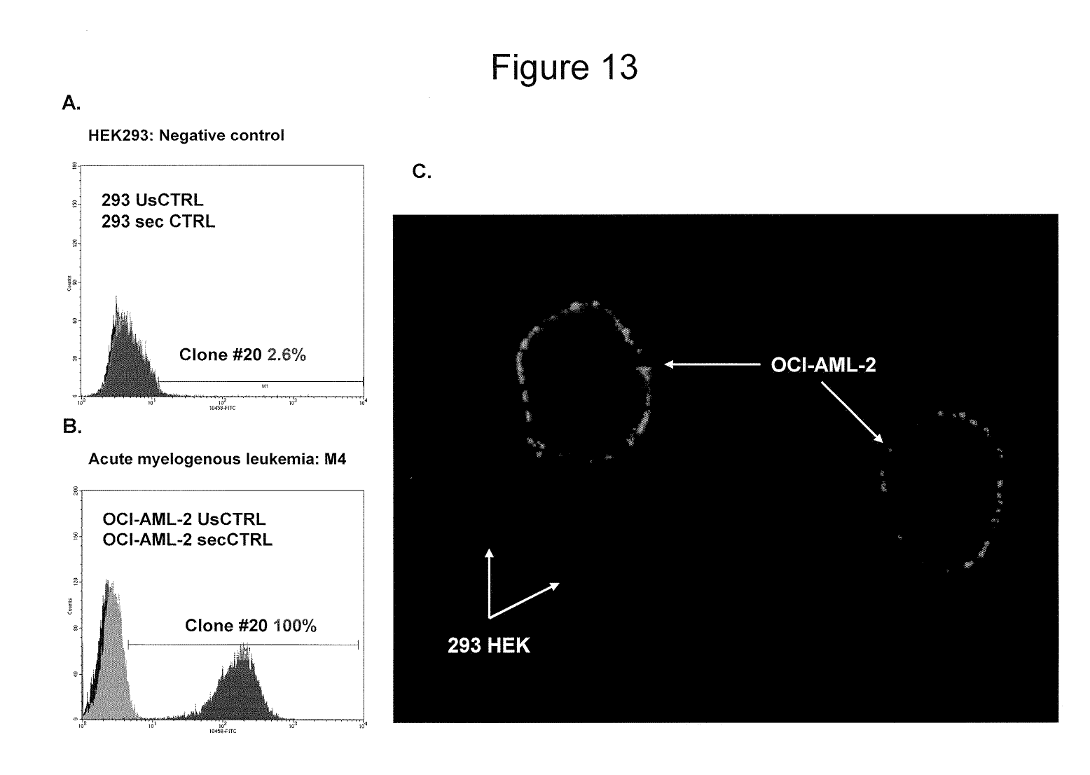

As can be seen in FIG. 13, 100% of the AML cells were positive by FACS analysis (FIG. 13B) and the presence of the fluorescent halo (indicated by white arrow) around the AML cells denotes the presence of KIRHy on the cell surface of OCI-AML-2 but not HEK-293 cells as detected by Clone #20 mAb.

D. Biological Activity

1. Cytotoxicity Analysis

Cytotoxicity analysis was performed using internalization of Mab-Zap (Advanced Targeting Systems, San Diego, Calif.) which is an anti-mouse Ig antibody conjugated to saponin (a ribosome inhibitor). Only by binding a primary antibody that is in turn internalized will this reagent kill. The saponin is conjugated to the secondary antibody via an acidic labile linkage that is cleaved upon entry into the endosome whereupon the toxin is released.

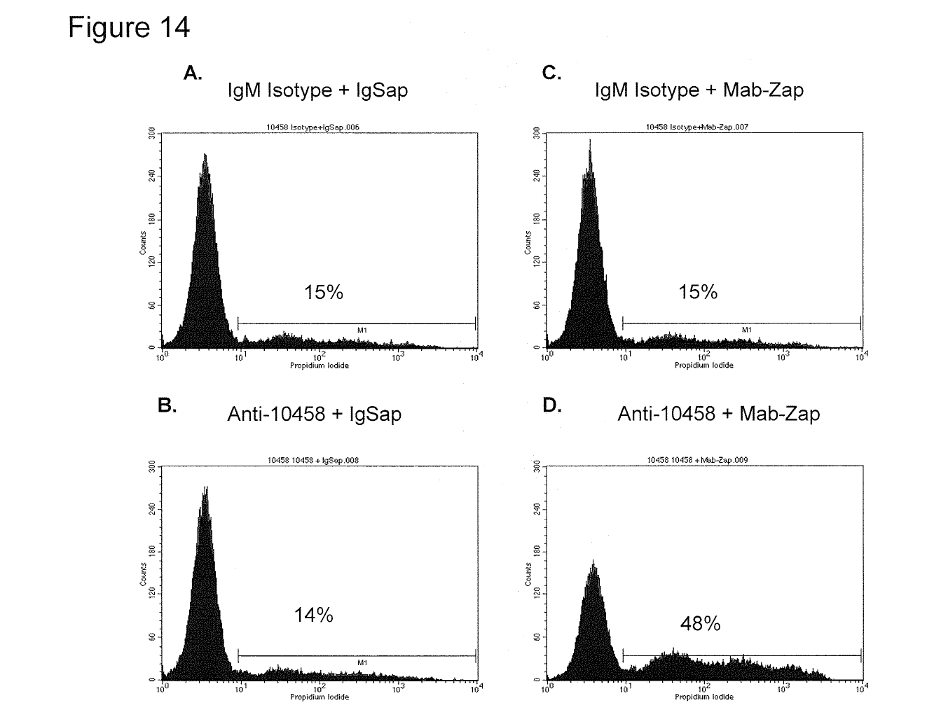

Cells were processed as described above for FACS analysis. Briefly, 1-2×10⁶ target OCI-AML-2 cells were aliquoted, pelleted, and resuspended in 100 μl of FACS buffer (1% BSA in PBS) to which 1 μg/10⁶ cells of antibody (treatments: IgM isotype control, Clone #20 mAb, IgSap, a non-specific antibody conjugated to saponin, and Mab-Zap, a mouse-specific antibody conjugated to saponin) was added and incubated for 20 min on ice. Excess antibody was removed by twice washing in FACS wash buffer (PBS). The cell pellet was resuspended in 500 μl FACS wash buffer and analyzed by FACS. Cells were resuspended in 500 μl medium, transferred to a 24-well plate and incubated at 37° C. for 4-6 hours. Killing was measured by FACS using internalization of propridium iodide (PI). PI is only able to enter cells if there is damage to the cell membrane (e.g. as a result of cell death). PI was used because it intercalates into DNA and has an emission spectrum in the FL3 range on the FACS machine. In this protocol, the proportion of cells with intercalated PI was counted. Only the internalized antibody will induce cell killing. The percentage noted on the histograms in FIG. 14 reflects the percent of cells killed by the treatment. The greatest percent killing was seen in the Clone #20+Mab-Zap treatment indicating that the Clone #20-KIRHy complex is internalized.

2. Complement-Dependent Cytotoxicity) CDC-Mediated Killing

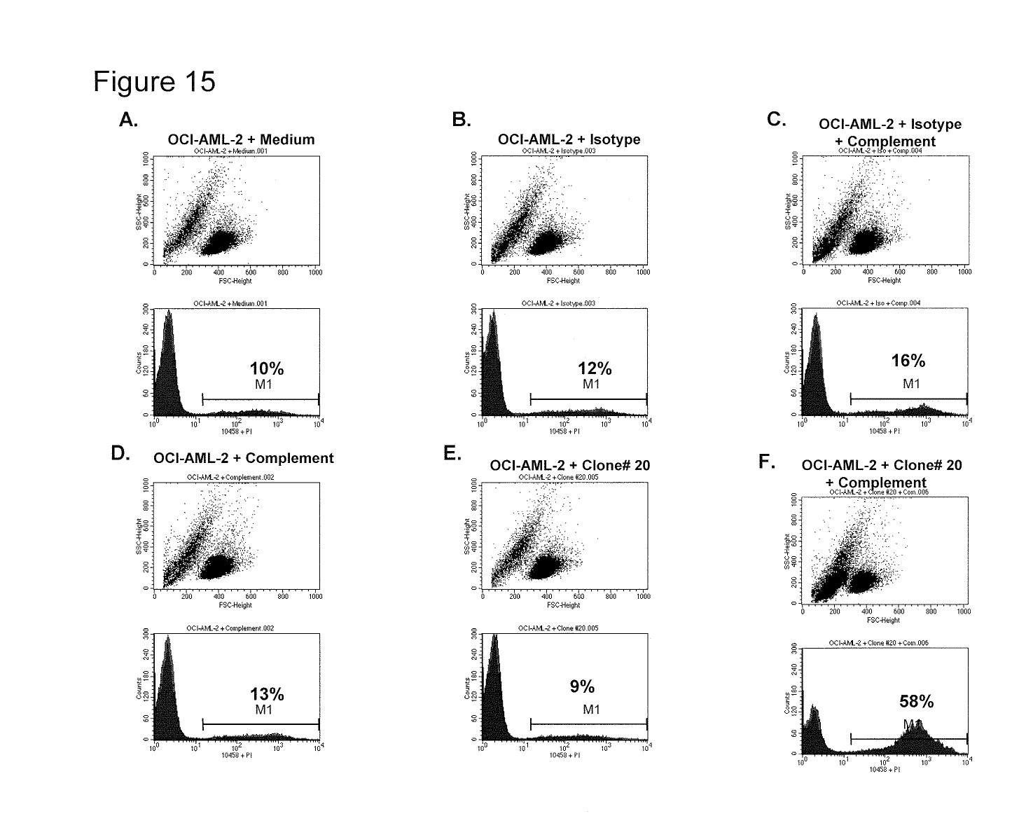

This CDC assay measures the ability of the Clone #20 mAb to fix complement on the surface of KIRHy antigen positive target cells. Cells were treated as described above for FACS analysis with the exception of resuspending the final cell pellet in 500 μl of medium and transferred to 24-well plates containing medium plus baby rabbit complement and incubated at 37° C. for 4-5 hours. Cell killing was measured by FACS analysis using internalization of PI. As can be seen in FIG. 15, the greatest CDC-mediated cell killing was in the Clone #20+ complement sample (FIG. 15F) indicating that Clone #20 can fix complement in response to KIRHy cell surface binding.

### Example 10

### Analysis of 10458-Fc Monoclonal Antibodies

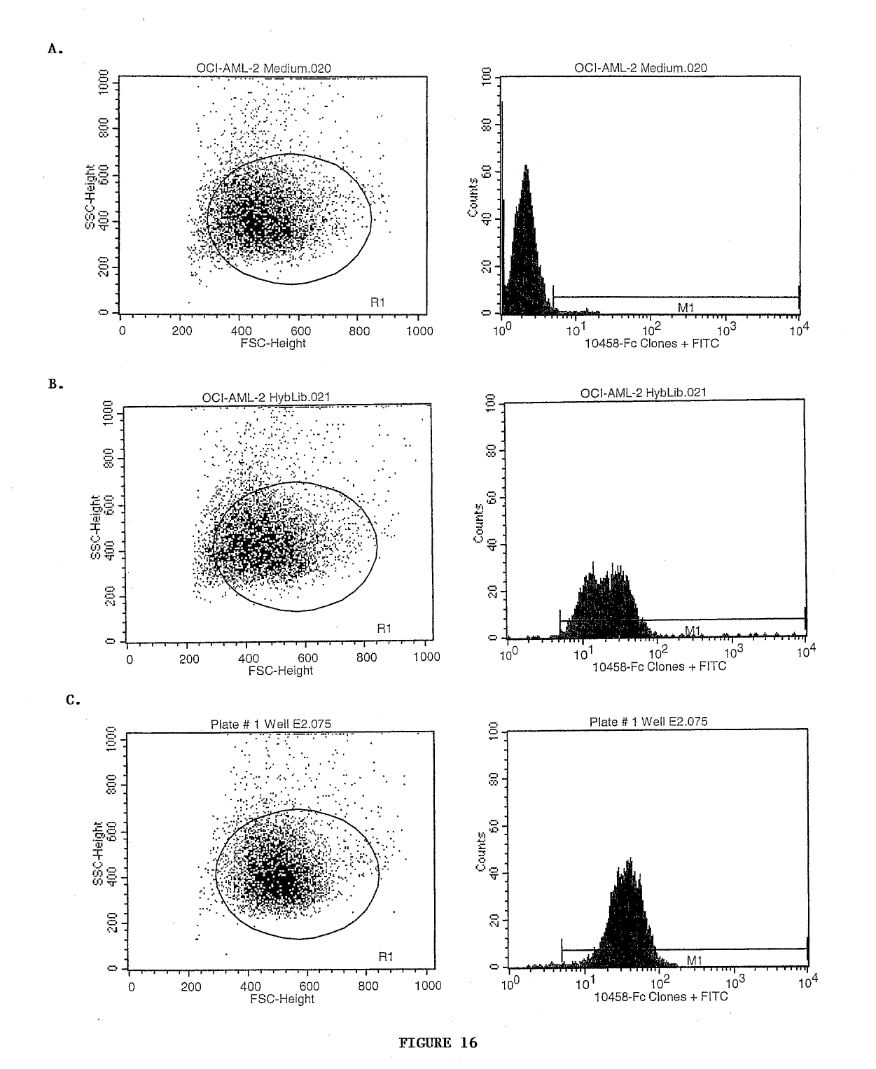

OCI-AML-2 cells were used to screen the 10458-Fc anti-KIRHy monoclonal antibodies using FACS analysis for positive clones. Briefly, 1×10⁶ OCI-AML-2 target cells were aliquoted, washed and resuspended in 100 μl FACS buffer (1% BSA in PBS). 50 μl of antibody conditioned supernatant (1 μg/10⁶ cells) was added to the resuspended cells and incubated on ice for 20 min. Excess antibody was removed by twice washing with FACS wash buffer and the cell pellet was again resuspended in 100 μl FACS buffer to which 100 μg/10⁶ cells of secondary antibody (goat anti-mouse IgG+IgM-FITC) was added. Excess antibody was removed by washing and the cell pellet was resuspended in 500 μl FACS wash buffer and analyzed by FACS. FIG. 16 shows a FACS histogram for 10458-Fc clone #75 (FIG. 16C) as compared to medium alone (FIG. 16A) and conditioned medium from the hybridization library (FIG. 16B). Table 8 displays the 10458-Fc mAbs that were positive by FACS analysis.

TABLE 8

| Clone No. | FACS Status | ATCC Deposit No. and Date |
|---|---|---|
| 1 | Positive | XXXXX; Xx/Xx/Xx |
| 2 | Positive | XXXXX; Xx/Xx/Xx |
| 3 | Positive | XXXXX; Xx/Xx/Xx |
| 4 | Positive | XXXXX; Xx/Xx/Xx |
| 5 | Positive | XXXXX; Xx/Xx/Xx |
| 6 | Positive | XXXXX; Xx/Xx/Xx |
| 7 | Positive | XXXXX; Xx/Xx/Xx |
| 8 | Positive | XXXXX; Xx/Xx/Xx |
| 9 | Positive | XXXXX; Xx/Xx/Xx |
| 10 | Positive | XXXXX; Xx/Xx/Xx |
| 11 | Positive | XXXXX; Xx/Xx/Xx |
| 12 | Positive | XXXXX; Xx/Xx/Xx |
| 13 | Positive | XXXXX; Xx/Xx/Xx |
| 14 | Positive | XXXXX; Xx/Xx/Xx |
| 15 | Positive | XXXXX; Xx/Xx/Xx |
| 16 | Positive | XXXXX; Xx/Xx/Xx |
| 17 | Positive | XXXXX; Xx/Xx/Xx |
| 18 | Positive | XXXXX; Xx/Xx/Xx |
| 19 | Positive | XXXXX; Xx/Xx/Xx |
| 20 | Positive | XXXXX; Xx/Xx/Xx |
| 21 | Positive | XXXXX; Xx/Xx/Xx |
| 22 | Positive | XXXXX; Xx/Xx/Xx |
| 23 | Positive | XXXXX; Xx/Xx/Xx |
| 24 | Positive | XXXXX; Xx/Xx/Xx |
| 25 | Positive | XXXXX; Xx/Xx/Xx |
| 26 | Positive | XXXXX; Xx/Xx/Xx |
| 27 | Positive | XXXXX; Xx/Xx/Xx |
| 28 | Positive | XXXXX; Xx/Xx/Xx |
| 29 | Positive | XXXXX; Xx/Xx/Xx |
| 30 | Positive | XXXXX; Xx/Xx/Xx |
| 31 | Positive | XXXXX; Xx/Xx/Xx |
| 32 | Positive | XXXXX; Xx/Xx/Xx |
| 33 | Positive | XXXXX; Xx/Xx/Xx |
| 34 | Positive | XXXXX; Xx/Xx/Xx |
| 35 | Positive | XXXXX; Xx/Xx/Xx |
| 36 | Positive | XXXXX; Xx/Xx/Xx |
| 37 | Positive | XXXXX; Xx/Xx/Xx |
| 38 | Positive | XXXXX; Xx/Xx/Xx |
| 39 | Positive | XXXXX; Xx/Xx/Xx |
| 40 | Positive | XXXXX; Xx/Xx/Xx |
| 41 | Positive | XXXXX; Xx/Xx/Xx |
| 42 | Positive | XXXXX; Xx/Xx/Xx |
| 43 | Positive | XXXXX; Xx/Xx/Xx |
| 44 | Positive | XXXXX; Xx/Xx/Xx |
| 45 | Positive | XXXXX; Xx/Xx/Xx |
| 46 | Positive | XXXXX; Xx/Xx/Xx |
| 47 | Positive | XXXXX; Xx/Xx/Xx |
| 48 | Positive | XXXXX; Xx/Xx/Xx |
| 49 | Positive | XXXXX; Xx/Xx/Xx |
| 50 | Positive | XXXXX; Xx/Xx/Xx |
| 51 | Positive | XXXXX; Xx/Xx/Xx |
| 52 | Positive | XXXXX; Xx/Xx/Xx |
| 53 | Positive | XXXXX; Xx/Xx/Xx |
| 54 | Positive | XXXXX; Xx/Xx/Xx |
| 55 | Positive | XXXXX; Xx/Xx/Xx |
| 56 | Positive | XXXXX; Xx/Xx/Xx |
| 57 | Positive | XXXXX; Xx/Xx/Xx |
| 58 | Positive | XXXXX; Xx/Xx/Xx |
| 59 | Positive | XXXXX; Xx/Xx/Xx |
| 60 | Positive | XXXXX; Xx/Xx/Xx |
| 61 | Positive | XXXXX; Xx/Xx/Xx |
| 62 | Positive | XXXXX; Xx/Xx/Xx |
| 63 | Positive | XXXXX; Xx/Xx/Xx |
| 64 | Positive | XXXXX; Xx/Xx/Xx |
| 65 | Positive | XXXXX; Xx/Xx/Xx |
| 66 | Positive | XXXXX; Xx/Xx/Xx |
| 67 | Positive | XXXXX; Xx/Xx/Xx |
| 68 | Positive | XXXXX; Xx/Xx/Xx |
| 69 | Positive | XXXXX; Xx/Xx/Xx |
| 70 | Positive | XXXXX; Xx/Xx/Xx |
| 71 | Positive | XXXXX; Xx/Xx/Xx |
| 72 | Positive | XXXXX; Xx/Xx/Xx |
| 73 | Positive | XXXXX; Xx/Xx/Xx |
| 74 | Positive | XXXXX; Xx/Xx/Xx |
| 75 | Positive | XXXXX; Xx/Xx/Xx |
| 76 | Positive | XXXXX; Xx/Xx/Xx |
| 77 | Positive | XXXXX; Xx/Xx/Xx |
| 78 | Positive | XXXXX; Xx/Xx/Xx |
| 79 | Positive | XXXXX; Xx/Xx/Xx |
| 80 | Positive | XXXXX; Xx/Xx/Xx |
| 81 | Positive | XXXXX; Xx/Xx/Xx |
| 82 | Positive | XXXXX; Xx/Xx/Xx |
| 83 | Positive | XXXXX; Xx/Xx/Xx |
| 84 | Positive | XXXXX; Xx/Xx/Xx |
| 85 | Positive | XXXXX; Xx/Xx/Xx |
| 86 | Positive | XXXXX; Xx/Xx/Xx |
| 87 | Positive | XXXXX; Xx/Xx/Xx |
| 88 | Positive | XXXXX; Xx/Xx/Xx |
| 89 | Positive | XXXXX; Xx/Xx/Xx |
| 90 | Positive | XXXXX; Xx/Xx/Xx |
| 91 | Positive | XXXXX; Xx/Xx/Xx |
| 92 | Positive | XXXXX; Xx/Xx/Xx |
| 93 | Positive | XXXXX; Xx/Xx/Xx |
| 94 | Positive | XXXXX; Xx/Xx/Xx |
| 95 | Positive | XXXXX; Xx/Xx/Xx |
| 96 | Positive | XXXXX; Xx/Xx/Xx |
| 97 | Positive | XXXXX; Xx/Xx/Xx |
| 98 | Positive | XXXXX; Xx/Xx/Xx |
| 99 | Positive | XXXXX; Xx/Xx/Xx |
| 100 | Positive | XXXXX; Xx/Xx/Xx |
| 101 | Positive | XXXXX; Xx/Xx/Xx |
| 102 | Positive | XXXXX; Xx/Xx/Xx |
| 103 | Positive | XXXXX; Xx/Xx/Xx |
| 104 | Positive | XXXXX; Xx/Xx/Xx |
| 105 | Positive | XXXXX; Xx/Xx/Xx |
| 106 | Positive | XXXXX; Xx/Xx/Xx |
| 107 | Positive | XXXXX; Xx/Xx/Xx |
| 109 | Positive | XXXXX; Xx/Xx/Xx |
| 110 | Positive | XXXXX; Xx/Xx/Xx |
| 111 | Positive | XXXXX; Xx/Xx/Xx |
| 112 | Positive | XXXXX; Xx/Xx/Xx |
| 113 | Positive | XXXXX; Xx/Xx/Xx |
| 114 | Positive | XXXXX; Xx/Xx/Xx |
| 115 | Positive | XXXXX; Xx/Xx/Xx |
| 116 | Positive | XXXXX; Xx/Xx/Xx |
| 117 | Positive | XXXXX; Xx/Xx/Xx |
| 119 | Positive | XXXXX; Xx/Xx/Xx |
| 120 | Positive | XXXXX; Xx/Xx/Xx |
| 121 | Positive | XXXXX; Xx/Xx/Xx |
| 122 | Positive | XXXXX; Xx/Xx/Xx |
| 123 | Positive | XXXXX; Xx/Xx/Xx |
| 124 | Positive | XXXXX; Xx/Xx/Xx |
| 125 | Positive | XXXXX; Xx/Xx/Xx |
| 126 | Positive | XXXXX; Xx/Xx/Xx |
| 127 | Positive | XXXXX; Xx/Xx/Xx |
| 128 | Positive | XXXXX; Xx/Xx/Xx |
| 129 | Positive | XXXXX; Xx/Xx/Xx |
| 130 | Positive | XXXXX; Xx/Xx/Xx |
| 131 | Positive | XXXXX; Xx/Xx/Xx |
| 132 | Positive | XXXXX; Xx/Xx/Xx |
| 134 | Positive | XXXXX; Xx/Xx/Xx |
| 135 | Positive | XXXXX; Xx/Xx/Xx |
| 136 | Positive | XXXXX; Xx/Xx/Xx |
| 138 | Positive | XXXXX; Xx/Xx/Xx |
| 140 | Positive | XXXXX; Xx/Xx/Xx |
| 141 | Positive | XXXXX; Xx/Xx/Xx |
| 142 | Positive | XXXXX; Xx/Xx/Xx |
| 144 | Positive | XXXXX; Xx/Xx/Xx |
| 145 | Positive | XXXXX; Xx/Xx/Xx |
| 146 | Positive | XXXXX; Xx/Xx/Xx |
| 147 | Positive | XXXXX; Xx/Xx/Xx |
| 148 | Positive | XXXXX; Xx/Xx/Xx |
| 149 | Positive | XXXXX; Xx/Xx/Xx |
| 151 | Positive | XXXXX; Xx/Xx/Xx |
| 152 | Positive | XXXXX; Xx/Xx/Xx |
| 155 | Positive | XXXXX; Xx/Xx/Xx |
| 156 | Positive | XXXXX; Xx/Xx/Xx |
| 157 | Positive | XXXXX; Xx/Xx/Xx |
| 158 | Positive | XXXXX; Xx/Xx/Xx |
| 159 | Positive | XXXXX; Xx/Xx/Xx |
| 161 | Positive | XXXXX; Xx/Xx/Xx |
| 162 | Positive | XXXXX; Xx/Xx/Xx |
| 163 | Positive | XXXXX; Xx/Xx/Xx |
| 164 | Positive | XXXXX; Xx/Xx/Xx |
| 165 | Positive | XXXXX; Xx/Xx/Xx |
| 166 | Positive | XXXXX; Xx/Xx/Xx |
| 167 | Positive | XXXXX; Xx/Xx/Xx |
| 168 | Positive | XXXXX; Xx/Xx/Xx |
| 169 | Positive | XXXXX; Xx/Xx/Xx |
| 170 | Positive | XXXXX; Xx/Xx/Xx |
| 171 | Positive | XXXXX; Xx/Xx/Xx |
| 172 | Positive | XXXXX; Xx/Xx/Xx |
| 173 | Positive | XXXXX; Xx/Xx/Xx |
| 174 | Positive | XXXXX; Xx/Xx/Xx |
| 175 | Positive | XXXXX; Xx/Xx/Xx |
| 176 | Positive | XXXXX; Xx/Xx/Xx |
| 177 | Positive | XXXXX; Xx/Xx/Xx |
| 178 | Positive | XXXXX; Xx/Xx/Xx |
| 179 | Positive | XXXXX; Xx/Xx/Xx |
| 180 | Positive | XXXXX; Xx/Xx/Xx |
| 181 | Positive | XXXXX; Xx/Xx/Xx |
| 182 | Positive | XXXXX; Xx/Xx/Xx |
| 184 | Positive | XXXXX; Xx/Xx/Xx |
| 185 | Positive | XXXXX; Xx/Xx/Xx |
| 186 | Positive | XXXXX; Xx/Xx/Xx |
| 188 | Positive | XXXXX; Xx/Xx/Xx |
| 191 | Positive | XXXXX; Xx/Xx/Xx |
| 192 | Positive | XXXXX; Xx/Xx/Xx |
| 193 | Positive | XXXXX; Xx/Xx/Xx |
| 195 | Positive | XXXXX; Xx/Xx/Xx |
| 197 | Positive | XXXXX; Xx/Xx/Xx |
| 198 | Positive | XXXXX; Xx/Xx/Xx |
| 199 | Positive | XXXXX; Xx/Xx/Xx |
| 200 | Positive | XXXXX; Xx/Xx/Xx |
| 203 | Positive | XXXXX; Xx/Xx/Xx |
| 204 | Positive | XXXXX; Xx/Xx/Xx |
| 205 | Positive | XXXXX; Xx/Xx/Xx |
| 206 | Positive | XXXXX; Xx/Xx/Xx |
| 207 | Positive | XXXXX; Xx/Xx/Xx |
| 208 | Positive | XXXXX; Xx/Xx/Xx |
| 209 | Positive | XXXXX; Xx/Xx/Xx |
| 210 | Positive | XXXXX; Xx/Xx/Xx |
| 211 | Positive | XXXXX; Xx/Xx/Xx |
| 212 | Positive | XXXXX; Xx/Xx/Xx |
| 213 | Positive | XXXXX; Xx/Xx/Xx |
| 217 | Positive | XXXXX; Xx/Xx/Xx |
| 218 | Positive | XXXXX; Xx/Xx/Xx |
| 221 | Positive | XXXXX; Xx/Xx/Xx |
| 223 | Positive | XXXXX; Xx/Xx/Xx |
| 224 | Positive | XXXXX; Xx/Xx/Xx |
| 225 | Positive | XXXXX; Xx/Xx/Xx |
| 226 | Positive | XXXXX; Xx/Xx/Xx |
| 227 | Positive | XXXXX; Xx/Xx/Xx |
| 228 | Positive | XXXXX; Xx/Xx/Xx |
| 229 | Positive | XXXXX; Xx/Xx/Xx |
| 230 | Positive | XXXXX; Xx/Xx/Xx |
| 231 | Positive | XXXXX; Xx/Xx/Xx |
| 232 | Positive | XXXXX; Xx/Xx/Xx |
| 233 | Positive | XXXXX; Xx/Xx/Xx |
| 234 | Positive | XXXXX; Xx/Xx/Xx |
| 235 | Positive | XXXXX; Xx/Xx/Xx |
| 236 | Positive | XXXXX; Xx/Xx/Xx |
| 237 | Positive | XXXXX; Xx/Xx/Xx |
| 238 | Positive | XXXXX; Xx/Xx/Xx |
| 238A | Positive | XXXXX; Xx/Xx/Xx |
| 239 | Positive | XXXXX; Xx/Xx/Xx |
| 244 | Positive | XXXXX; Xx/Xx/Xx |
| 251 | Positive | XXXXX; Xx/Xx/Xx |
| 253 | Positive | XXXXX; Xx/Xx/Xx |
| 254 | Positive | XXXXX; Xx/Xx/Xx |
| 255 | Positive | XXXXX; Xx/Xx/Xx |
| 256 | Positive | XXXXX; Xx/Xx/Xx |
| 257 | Positive | XXXXX; Xx/Xx/Xx |
| 258 | Positive | XXXXX; Xx/Xx/Xx |
| 260 | Positive | XXXXX; Xx/Xx/Xx |
| 261 | Positive | XXXXX; Xx/Xx/Xx |
| 262 | Positive | XXXXX; Xx/Xx/Xx |
| 263 | Positive | XXXXX; Xx/Xx/Xx |
| 265 | Positive | XXXXX; Xx/Xx/Xx |
| 266 | Positive | XXXXX; Xx/Xx/Xx |
| 267 | Positive | XXXXX; Xx/Xx/Xx |
| 268 | Positive | XXXXX; Xx/Xx/Xx |
| 269 | Positive | XXXXX; Xx/Xx/Xx |
| 270 | Positive | XXXXX; Xx/Xx/Xx |
| 500 | Positive | XXXXX; Xx/Xx/Xx |
| 501 | Positive | XXXXX; Xx/Xx/Xx |
| 502 | Positive | XXXXX; Xx/Xx/Xx |
| 503 | Positive | XXXXX; Xx/Xx/Xx |
| 504 | Positive | XXXXX; Xx/Xx/Xx |
| 505 | Positive | XXXXX; Xx/Xx/Xx |
| 506 | Positive | XXXXX; Xx/Xx/Xx |
| 507 | Positive | XXXXX; Xx/Xx/Xx |
| 508 | Positive | XXXXX; Xx/Xx/Xx |
| 509 | Positive | XXXXX; Xx/Xx/Xx |
| 510 | Positive | XXXXX; Xx/Xx/Xx |
| 511 | Positive | XXXXX; Xx/Xx/Xx |
| 600 | Positive | XXXXX; Xx/Xx/Xx |
| 601 | Positive | XXXXX; Xx/Xx/Xx |
| 602 | Positive | XXXXX; Xx/Xx/Xx |
| 603 | Positive | XXXXX; Xx/Xx/Xx |
| 604 | Positive | XXXXX; Xx/Xx/Xx |
| 605 | Positive | XXXXX; Xx/Xx/Xx |
| 606 | Positive | XXXXX; Xx/Xx/Xx |
| 607 | Positive | XXXXX; Xx/Xx/Xx |
| 608 | Positive | XXXXX; Xx/Xx/Xx |
| 609 | Positive | XXXXX; Xx/Xx/Xx |
| 610 | Positive | XXXXX; Xx/Xx/Xx |
| 611 | Positive | XXXXX; Xx/Xx/Xx |
| 612 | Positive | XXXXX; Xx/Xx/Xx |

The 10458-Fc mAbs were deposited under the terms of the Budapest Treaty with the American Type Culture Collection (ATCC), 10801 University Blvd., Manassas, Va. 20110-2209, USA on the date and given the corresponding deposit number listed in “ATCC Deposit No. and Date” as shown in Table 8 above.

These deposits were made under the provisions of the Budapest Treaty on the International Recognition of the Deposit of Microorganisms for the Purpose of Patent Procedure and the Regulations thereunder (Budapest Treaty). This assures maintenance of a viable culture of the deposit for 30 years from the date of the deposit. The deposits will be made available by ATCC under the terms of the Budapest Treaty, and subject to an agreement between Nuvelo, Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 U.S.C. §122 and the Commissioner's rules pursuant thereto (including 37 C.F.R. §1.14 with particular reference to 886 OG 638).

The assignee of the present application has agreed that if a culture of the materials on deposit should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

### Example 11

### Toxin-Conjugated KIRHy-Specific Antibodies

Antibodies to KIRHy are conjugated to toxins and the effect of such conjugates in animal models of cancer is evaluated. Chemotherapeutic agents, such as calicheamycin and carboplatin, or toxic peptides, such as ricin toxin, are used in this approach. Antibody-toxin conjugates are used to target cytotoxic agents specifically to cells bearing the antigen. The antibody-toxin binds to these antigen-bearing cells, becomes internalized by receptor-mediated endocytosis, and subsequently destroys the targeted cell. In this case, the antibody-toxin conjugate targets KIRHy-expressing cells, such as AML cells, and deliver the cytotoxic agent to the tumor resulting in the death of the tumor cells.

One such example of a toxin that may be conjugated to an antibody is carboplatin. The mechanism by which this toxin is conjugated to antibodies is described in Ota et al., Asia-Oceania J. Obstet. Gynaecol. 19: 449-457 (1993). The cytotoxicity of carboplatin-conjugated KIRHy-specific antibodies is evaluated in vitro, for example, by incubating KIRHy-expressing target cells (such as the AML cell line) with various concentrations of conjugated antibody, medium alone, carboplatin alone, or antibody alone. The antibody-toxin conjugate specifically targets and kills cells bearing the KIRHy antigen, whereas, cells not bearing the antigen, or cells treated with medium alone, carboplatin alone, or antibody alone, show no cytotoxicity.

The antitumor efficacy of carboplatin-conjugated KIRHy-specific antibodies is demonstrated in in vivo murine tumor models. Five to six week old, athymic nude mice are engrafted with tumors subcutaneously or through intravenous injection. Mice are treated with the KIRHy-carboplatin conjugate or with a non-specific antibody-carboplatin conjugate. Tumor xenografts in the mouse bearing the KIRHy antigen are targeted and bound to by the KIRHy-carboplatin conjugate. This results in tumor cell killing as evidenced by tumor necrosis, tumor shrinkage, and increased survival of the treated mice.

Other toxins are conjugated to KIRHy-specific antibodies using methods known in the art. An example of a toxin conjugated antibody in human clinical trials is CMA-676, an antibody to the CD33 antigen in AML which is conjugated with calicheamicin toxin (Larson, Semin. Hematol. 38(Suppl 6):24-31 (2001)).

### Example 12

### Radio-Immunotherapy Using KIRHy-Specific Antibodies

Animal models are used to assess the effect of antibodies specific to KIRHy as vectors in the delivery of radionuclides in radio-immunotherapy to treat AML and histiocytic lymphoma. Human tumors are propagated in 5-6 week old athymic nude mice by injecting a carcinoma cell line or tumor cells subcutaneously. Tumor-bearing animals are injected intravenously with radio-labeled anti-KIRHy antibody (labeled with 30-40 μCi of ¹³¹I, for example) (Behr, et al., Int. J. Cancer 77: 787-795 (1988)). Tumor size is measured before injection and on a regular basis (i.e. weekly) after injection and compared to tumors in mice that have not received treatment. Anti-tumor efficacy is calculated by correlating the calculated mean tumor doses and the extent of induced growth retardation. To check tumor and organ histology, animals are sacrificed by cervical dislocation and autopsied. Organs are fixed in 10% formalin, embedded in paraffin, and thin sectioned. The sections are stained with hematoxylin-eosin.

### Example 13

### Immunotherapy Using KIRHy-Specific Antibodies

Animal models are used to evaluate the effect of KIRHy-specific antibodies as targets for antibody-based immunotherapy using monoclonal antibodies. Human AML cells are injected into the tail vein of 5-6 week old nude mice whose natural killer cells have been eradicated. To evaluate the ability of KIRHy-specific antibodies in preventing tumor growth, mice receive an intraperitoneal injection with KIRHy-specific antibodies either 1 or 15 days after tumor inoculation followed by either a daily dose of 20 μg or 100 μg once or twice a week, respectively (Ozaki, et al., Blood 90:3179-3186 (1997)). Levels of human IgG (from the immune reaction caused by the human tumor cells) are measured in the murine sera by ELISA.

The effect of KIRHy-specific antibodies on the proliferation of AML cells is examined in vitro using a ³H-thymidine incorporation assay (Ozaki et al., supra). Cells are cultured in 96-well plates at 1×10⁵ cells/ml in 100 μl/well and incubated with various amounts of KIRHy antibody or control IgG (up to 100 μg/ml) for 24 h. Cells are incubated with 0.5 μCi ³H-thymidine (New England Nuclear, Boston, Mass.) for 18 h and harvested onto glass filters using an automatic cell harvester (Packard, Meriden, Conn.). The incorporated radioactivity is measured using a liquid scintillation counter.

The cytotoxicity of the KIRHy monoclonal antibody is examined by the effect of complements on AML cells using a ⁵¹Cr-release assay (Ozaki et al., supra). AML cells are labeled with 0.1 mCi ⁵¹Cr-sodium chromate at 37° C. for 1 h. ⁵¹Cr-labeled cells are incubated with various concentrations of KIRHy monoclonal antibody or control IgG on ice for 30 min. Unbound antibody is removed by washing with medium. Cells are distributed into 96-well plates and incubated with serial dilutions of baby rabbit complement at 37° C. for 2 h. The supernatants are harvested from each well and the amount of ⁵¹Cr released is measured using a gamma counter. Spontaneous release of ⁵¹Cr is measured by incubating cells with medium alone, whereas maximum ⁵¹Cr release is measured by treating cells with 1% NP-40 to disrupt the plasma membrane. Percent cytotoxicity is measured by dividing the difference of experimental and spontaneous ⁵¹Cr release by the difference of maximum and spontaneous ⁵¹Cr release.

Antibody-dependent cell-mediated cytotoxicity (ADCC) for the KIRHy monoclonal antibody is measured using a standard 4 h ⁵¹Cr-release assay (Ozaki et al., supra). Splenic mononuclear cells from SCID mice are used as effector cells and cultured with or without recombinant interleukin-2 (for example) for 6 days. ⁵¹Cr-labeled target AML cells (1×10⁴ cells) are placed in 96-well plates with various concentrations of anti-KIRHy monoclonal antibody or control IgG. Effector cells are added to the wells at various effector to target ratios (12.5:1 to 50:1). After 4 h, culture supernatants are removed and counted in a gamma counter. The percentage of cell lysis is determined as above.

### Example 14

### KIRHy-Specific Antibodies as Immunosuppressants

Animal models are used to assess the effect of KIRHy-specific antibodies block signaling through the KIRHy receptor to suppress autoimmune diseases, such as arthritis or other inflammatory conditions, or rejection of organ transplants. Immunosuppression is tested by injecting mice with horse red blood cells (HRBCs) and assaying for the levels of HRBC-specific antibodies (Yang, et al., Int. Immunopharm. 2:389-397 (2002)). Animals are divided into five groups, three of which are injected with anti-KIRHy antibodies for 10 days, and 2 of which receive no treatment. Two of the experimental groups and one control group are injected with either Earle's balanced salt solution (EBSS) containing 5−10×10⁷ HRBCs or EBSS alone. Anti-KIRHy antibody treatment is continued for one group while the other groups receive no antibody treatment. After 6 days, all animals are bled by retro-orbital puncture, followed by cervical dislocation and spleen removal. Splenocyte suspensions are prepared and the serum is removed by centrifugation for analysis.

Immunosupression is measured by the number of B cells producing HRBC-specific antibodies. The Ig isotype (for example, IgM, IgG1, IgG2, etc.) is determined using the IsoDetect™ Isotyping kit (Stratagene, La Jolla, Calif.). Once the Ig isotype is known, murine antibodies against HRBCs are measured using an ELISA procedure. 96-well plates are coated with HRBCs and incubated with the anti-HRBC antibody-containing sera isolated from the animals. The plates are incubated with alkaline phosphatase-labeled secondary antibodies and color development is measured on a microplate reader (SPECTRAmax 250, Molecular Devices) at 405 nm using p-nitrophenyl phosphate as a substrate.

Lymphocyte proliferation is measured in response to the T and B cell activators concanavalin A and lipopolysaccharide, respectively (Jiang, et al., J. Immunol. 154:3138-3146 (1995). Mice are randomly divided into 2 groups, 1 receiving anti-KIRHy antibody therapy for 7 days and 1 as a control. At the end of the treatment, the animals are sacrificed by cervical dislocation, the spleens are removed, and splenocyte suspensions are prepared as above. For the ex vivo test, the same number of splenocytes are used, whereas for the in vivo test, the anti-KIRHy antibody is added to the medium at the beginning of the experiment. Cell proliferation is also assayed using the ³H-thymidine incorporation assay described above (Ozaki, et al., Blood 90: 3179 (1997)).

### Example 15

### Cytokine Secretion in Response to KIRHy Peptide Fragments

Assays are carried out to assess activity of fragments of the KIRHy protein, such as the Ig domain, to stimulate cytokine secretion and to stimulate immune responses in NK cells, B cells, T cells, and myeloid cells. Such immune responses can be used to stimulate the immune system to recognize and/or mediate tumor cell killing or suppression of growth. Similarly, this immune stimulation can be used to target bacterial or viral infections. Alternatively, fragments of the KIRHy that block activation through the KIRHy receptor may be used to block immune stimulation in natural killer (NK), B, T, and myeloid cells.

Fusion proteins containing fragments of the KIRHy, such as the Ig domain (KIRHy-Ig), are made by inserting a CD33 leader peptide, followed by a KIRHy domain fused to the Fc region of human IgG1 into a mammalian expression vector, which is stably transfected into NS-1 cells, for example. The fusion proteins are secreted into the culture supernatant, which is harvested for use in cytokine assays, such as interferon-γ (IFN-γ) secretion assays (Martin, et al., J. Immunol. 167:3668-3676 (2001)).

PBMCs are activated with a suboptimal concentration of soluble CD3 and various concentrations of purified, soluble anti-KIRHy monoclonal antibody or control IgG. For KIRHy-Ig cytokine assays, anti-human Fc Ig at 5 or 20 μg/ml is bound to 96-well plates and incubated overnight at 4° C. Excess antibody is removed and either KIRHy-Ig or control Ig is added at 20-50 μg/ml and incubated for 4 h at room temperature. The plate is washed to remove excess fusion protein before adding cells and anti-CD3 to various concentrations. Supernatants are collected after 48 h of culture and IFN-γ levels are measured by sandwich ELISA, using primary and biotinylated secondary anti-human IFN-γ antibodies as recommended by the manufacturer.

### Example 16

### Tumor Imaging Using KIRHy-Specific Antibodies

KIRHy-specific antibodies are used for imaging KIRHy-expressing cells in vivo. Six-week-old athymic nude mice are irradiated with 400 rads from a cesium source. Three days later the irradiated mice are inoculated with 4×10⁷ RA1 cells and 4×10⁶ human fetal lung fibroblast feeder cells subcutaneously in the thigh. When the tumors reach approximately 1 cm in diameter, the mice are injected intravenously with an inoculum containing 100 μCi/10 μg of ¹³¹I-labeled KIRHy-specific antibody. At 1, 3, and 5 days postinjection, the mice are anesthetized with a subcutaneous injection of 0.8 mg sodium pentobarbital. The immobilized mice are then imaged in a prone position with a Spectrum 91 camera equipped with a pinhole collimator (Raytheon Medical Systems; Melrose Park, Ill.) set to record 5,000 to 10,000 counts using the Nuclear MAX Plus image analysis software package (MEDX Inc.; Wood Dale, Ill.) (Hornick, et al., Blood 89:4437-4447 (1997)).

### Example 17

### In Vivo Tumor Models

The tumor suppressing activity of KIRHy targeting molecules is tested by taking groups of 4-10 nude, athymic male mice are injected subcutaneously with 10⁶ cells, either a control (M12 pcDNA), KIRHy expressing clones, or low expressing clones (Spenger et al., Cancer Research 59:2370-2375 (1999), incorporated herein by reference in its entirety). The clones the lowest levels of KIRHy are used as the comparison benchmark. Mice are monitored for 8 weeks for weight gain/loss and tumor formation. Tumor volume is calculated using the formula (l×w²)/2 (where l=length and w=width of the tumor) (Id.).

Statistical analysis using the Kruskal-Wallis method for comparing tumor formation, and the Mann-Whitney U test for comparing tumor volume are performed to determine any statistical significance amongst groups.

After 8 weeks, the mice are sacrificed, and the tumors removed and digested with 0.1% collagenase (Type I) and 50 μg/ml DNase (Worthington Biochemical Corp., Freehold, N.J.). Dispersed cells are plated in ITS medium/5% FBS at %% CO₂ at 37° C. for 24 hours to allow attachment. After 24 hours, the cultures are switched to serum-free medium. The cells are split, the media and RNA collected, and Western immunoblots and Northern blots are done to detect KIRHy.

### Example 18

### In Vitro Assay of Cell Proliferation and Migration

The effect of KIRHy-specific antibodies or therapeutic peptides on the proliferation of AML cells is examined in vitro using a ³H-thymidine incorporation assay (Ozaki et al., Blood 90:3179-3186 (1997), herein incorporated by reference in its entirety. Tumor cells are cultured in 96-well plates at 1×10⁵ cells/ml in 100 μl/well and incubated with various amounts of antibody or control IgG (up to 100 μg/ml) for 24 h. Cells are incubated with 0.5 μCi ³H-thymidine (New England Nuclear, Boston, Mass.) for 18 h and harvested onto glass filters using an automatic cell harvester (Packard, Meriden, Conn.). The incorporated radioactivity is measured using a liquid scintillation counter.

Cell migration is conducted in 24-well, 6.5-mm internal diameter Transwell cluster plates (Corning Costar, Cambridge, Mass.). Briefly, 10⁵ cells/75 μl are loaded onto fibronectin (5 μM)-coated polycarbonate membranes (8-μm pore size) separating two chambers of a transwell (Tai et al., Blood 99:1419-1427 (2002), herein incorporated by reference in its entirety. Medium with or without anti-KIRHy antibodies is added to the lower chamber of the Transwell cluster plates. After 8-16 h, cells migrating to the lower chamber are counted using a Coulter counter ZBII (Beckman Coulter) and by hemacytometer.

### Example 19

### Clinical Trials for AML

For examples of AML clinical trials using immunotherapy, see Larson, Semin. Hematol. 38 (Suppl 6): 24-31 (2001), herein incorporated by reference in its entirety. For examples of clinical trials using small molecules, see Fiedler et al., Blood 102:2763-2767 (2003), O'Farrell et al., Clin. Cancer Res. 9:5465-5476 (2003), Ohno et al., J. Clin. Oncol. 8:1907-1912 (1990), all of which are herein incorporated by reference in their entirety.

Patients are admitted with AML who have adequate hepatic and renal function at study entry and are confirmed to be KIRHy-positive. Patients are treated with a physiological dose of anti-KIRHy targeting agent and are examined for adverse events, toxicity and for end response criteria. Complete blood counts including peripheral blood counts are taken daily for the first four days followed by every 2 days for the first 2 weeks and then twice a week for the remainder of the study. Bone marrow examinations are taken after the first week and then every 2 weeks for the remainder of the study. Safety assessments include the evaluation of adverse events and vital signs, hematologic tests, biochemical tests, urinalysis, and physical examination. Toxicity is graded in accordance with the Common Toxicity Criteria of the National Cancer Institute. Response criteria include complete remission, using conventional criteria, and complete remission without full platelet recovery, but no longer dependent on platelet transfusions. The criteria include bone marrow consisting of less than 5% blasts, peripheral blood free of blasts, hemoglobin greater than or equal to 9 g/dL, absolute neutrophil count of greater than or equal to 1500/μl, transfusion independence, and platelet count greater than or equal to 100,000/μl.

We claim:

1. A pharmaceutical composition comprising an anti-KIRHy antibody specific for cells that cause a myeloproliferative disorder, wherein said antibody specifically binds to a KIRHy polypeptide or immunogenic fragment thereof.

2. The pharmaceutical composition of claim 1, wherein said antibody is a monoclonal anti-KIRHy antibody or antigen-binding fragment thereof.

3. The pharmaceutical composition of claim 1, wherein said antibody is a humanized anti-KIRHy antibody or antigen-binding fragment thereof.

4. The pharmaceutical composition of claim 1, wherein said antibody is labeled with a toxin.

5. The pharmaceutical composition of claim 1, wherein said antibody is labeled with a radioisotope.

6. The pharmaceutical composition of claim 1, wherein said antibody is administered in an amount effective to kill or inhibit the growth of cells that cause a myeloproliferative disorder.

7. The pharmaceutical composition of claim 1, wherein said myeloproliferative disorder is selected from the group consisting of leukemia, acute myelogenous leukemia (AML), chronic myelogenous leukemia (CML), plasmacytoma, and histiocytic lymphoma.

8. A method of targeting a KIRHy protein on KIRHy-expressing cells that cause a myeloproliferative disorder, comprising the step of administering a pharmaceutical composition to said cells in an amount effective to target said cells, wherein said composition is an anti-KIRHy antibody.

9. A method of killing or inhibiting the growth of KIRHy-expressing cells that cause a myeloproliferative disorder, comprising the step of administering a pharmaceutical composition to said cells in an amount effective to kill or inhibit the growth of said cells, wherein said composition is an anti-KIRHy antibody.

10. (canceled)

11. A method of killing or inhibiting the growth of KIRHy-expressing cells that cause a myeloproliferative disorder, comprising the step of administering a pharmaceutical composition to said cells in an amount effective to kill or inhibit the growth of said cells, wherein said composition comprises a nucleic acid encoding a KIRHy polypeptide, or fragment thereof, within a recombinant vector.

12. A method of killing or inhibiting the growth of KIRHy-expressing cells that cause a myeloproliferative disorder, comprising the step of administering a pharmaceutical composition to said cells in an amount effective to kill or inhibit the growth of said cells, wherein said composition comprises an antigen-presenting cell comprising a nucleic acid encoding a KIRHy polypeptide, or fragment thereof, within a recombinant vector.

13. A method of killing or inhibiting the growth of KIRHy-expressing cells that cause a myeloproliferative disorder, comprising the step of administering a pharmaceutical composition to said cells in an amount effective to kill or inhibit the growth of said cells, wherein said composition comprises a small molecule that specifically binds to a KIRHy polypeptide, or fragment thereof.

14. A method of killing or inhibiting the growth of KIRHy-expressing cells that cause a myeloproliferative disorder, comprising the step of administering a pharmaceutical composition to said cells in an amount effective to kill or inhibit the growth of said cells, wherein said composition comprises a non-KIRHy polypeptide, or fragment thereof, that specifically binds to a KIRHy polypeptide or fragment thereof.

15. The method according to any one of claims 8, 9, 11, 12, 13, or 14, wherein said myeloproliferative disorder is selected from the group consisting of leukemia, acute myelogenous leukemia (AML), chronic myelogenous leukemia (CML), plasmacytoma, and histiocytic lymphoma.

16. The method according to any one of claims 8, 9, 11, 12, 13, or 14, wherein said cells are contacted with a second therapeutic agent.

17. The method according to any one of claims 8, 9, 11, 12, 13, or 14, wherein said pharmaceutical composition is administered in a sterile preparation together with a pharmaceutically acceptable carrier.

18. The method according to claim 8 or 9, wherein said anti-KIRHy antibody composition is administered in an amount to achieve a dosage range from about 0.1 to about 10 mg/kg body weight.

19. The method according to claim 8 or 9, wherein said anti-KIRHy antibody composition is a monoclonal antibody or antigen-binding fragment thereof.

20. The method according to claim 8 or 9, wherein said anti-KIRHy antibody composition is a humanized antibody or antigen-binding fragment thereof.

21. A method of diagnosing a myeloproliferative disorder comprising the steps of:
a) detecting or measuring the expression of KIRHy in or on a cell; and
b) comparing said expression to normal tissue.

22. The method according to claim 21, wherein said expression comprises KIRHy mRNA expression.

23. The method according to claim 21, wherein said expression comprises KIRHy protein expression.

24. The method according to claim 21, wherein said expression is detected or measured using a nucleic acid probe specific for a KIRHy nucleic acid.

25. The method according to claim 21, wherein said expression is detected or measured using anti-KIRHy antibodies.

26. The method according to claim 21, wherein said myeloproliferative disorder is selected from the group consisting of leukemia, acute myelogenous leukemia (AML), chronic myelogenous leukemia (CML), plasmacytoma, and histiocytic lymphoma.

27. An isolated polynucleotide comprising a nucleotide sequence selected from the group consisting of SEQ ID NO: 12, 16, 20, 22, 38, 42 and 50.

28. The polynucleotide of claim 27 which is a DNA sequence.

29. A vector comprising the polynucleotide of claim 27.

30. An expression vector comprising the polynucleotide of claim 27.

31. An isolated host cell genetically engineered to comprise the polynucleotide of claim 27.

32. An isolated host cell genetically engineered to comprise the polynucleotide of claim 27 operatively associated with a regulatory sequence that modulates expression of the polynucleotide in the host cell.

33. An isolated polypeptide comprising a polypeptide sequence selected from the group consisting of SEQ ID NO: 13, 17, 21, 23, 39, 43 and 51.

34. A composition comprising the polypeptide of claim 33 and a carrier.

35. An isolated antibody that specifically binds a polypeptide sequence selected from the group consisting of SEQ ID NO: 11, 13, 17, 21, 23, 37, 39, 43, and 51.

36. The antibody of claim 35, wherein said antibody comprises a monoclonal antibody or antibody fragment thereof.

37. The antibody of claim 35, wherein said antibody comprises a polyclonal antibody of antibody fragment thereof.

38. The antibody of claim 35, wherein said antibody comprises a humanized antibody or antibody fragment thereof.

39. The antibody of claim 35, wherein said antibody is 10458a.

40. The antibody of claim 35, wherein said antibody is the anti-KIRHy monoclonal antibody Clone #20.

41. The antibody of claim 35, wherein said antibody is selected from the monoclonal antibodies listed in Table 8.
